(19) United States
(12) Patent Application Publication
(10) Pub. No.: US 2007/0185176 A1
(43) Pub. Date: Aug. 9, 2007

(54) Heparanase inhibitors and uses thereof

(75) Inventors: Joel M. Van Gelder, Jerusalem (IL); Yochai Basel, Rehovot (IL); Boris O. Kraiz, Ramla (IL); Orly Mouallem, Jerusalem (IL); Daphna Miron, Rehovot (IL); Nina Gur-Arie, Rishon LeZion (IL); Joseph Klein, Haifa (IL)

Correspondence Address: BROWDY AND NEIMARK, P.L.L.C.;624 NINTH STREET, NW, SUITE 300, WASHINGTON, DC, 20001-5303, US

(73) Assignee: Insight Bioharmaceuticals Ltd., Rehovot (IL)

(21) Appl. No.: 10/588,554

(22) PCT Filed: Feb. 6, 2005
(86) PCT No.: PCT/IL05/00149

§ 371 Date: Aug. 7, 2006

Related U.S. Application Data
(60) Provisional application No. 60/541,904, filed on Feb. 6, 2004.

(51) Int. Cl. A61K 31/425

(52) U.S. Cl. 514/372; 514/676; 514/741

(57) ABSTRACT

The invention provides heparanase inhibitors suitable for treatment of diseases and disorders caused by or associated with heparanase catalytic activity such as cancer, inflammatory disorders and autoimmune diseases.

### FIELD AND BACKGROUND OF THE INVENTION

The present invention relates to heparanase inhibitors, and to their use in the treatment of diseases and- disorders caused by or associated with heparanase catalytic activity such as cancer, inflammatory disorders and autoimmune diseases.

Heparan sulfate proteoglycans (HSPGs) are ubiquitous macromolecules associated with the cell surface and with the extracellular matrix (ECM) of various tissues. They consist of a protein core to which several linear heparan sulfate (HS) chains are covalently attached. Studies on the involvement of ECM molecules in cell attachment, growth and differentiation revealed a central role of HSPGs in embryonic morphogenesis, angiogenesis, neurite outgrowth, tissue repair, and metastasis. HSPGs are also prominent components of blood vessels. In capillaries they are found mainly in the subendothelial basement membrane, where they support proliferating and migrating endothelial cells and stabilize the structure of the capillary wall.

Several cellular enzymes such as collagenase IV, plasminogen activator, cathepsin B, and elastase are thought to be involved in the degradation of basement membrane. Another enzyme of this type is heparanase, an endo-β-D-glucuronidase that cleaves HS at specific intrachain sites (Nakajima et al., 1984). Heparanase released from cells removes HS molecules from the basement membrane resulting in increase of basement membrane permeability. Heparanase also facilitates proteolytic degradation of the core structural components such as type IV collagen in collaboration with gelatinases. Thus, blood-borne cells accomplish penetration through the basement membrane. In fact, HS catabolism is observed in wound repair, inflammation, and in diabetes.

Expression of heparanase was found to correlate with the metastatic potential of mouse lymphoma (Vlodavsky et al., 1983), fibrosarcoma and melanoma cells (Nakajima et al., 1988). Similar correlation was observed in human breast, colon, bladder, prostate, and liver carcinomas (Vlodavsky et al., 1999). Moreover, elevated levels of heparanase were detected in sera of metastatic tumor bearing animals (Nakajima et al., 1988) and of cancer patients, in urine of highly metastatic patients (Vlodavsky et al., 1997), and in tumor biopsies (Vlodavsky et al., 1988). Treatment of experimental animals with heparanase substrates or inhibitors (e.g., non-anticoagulant species of low molecular weight heparin and polysulfated saccharides) considerably reduced the incidence of lung metastases induced by B16-F10 melanoma, pancreatic adenocarcinoma, Lewis lung carcinoma, and mammary adenocarcinoma cells (Vlodavsky et al., 1994; Nakajima et al., 1988; Parish et al., 1987; Lapierre et al., 1996), indicating that heparanase inhibitors may inhibit tumor cell invasion and metastasis.

Heparanase is involved also in primary tumor angiogenesis. Most primary solid tumors (1-2 mm diameter) obtain their oxygen and nutrient supply through a passive diffusion from pre-existing blood vessels, however the increase in their mass beyond this size requires angiogenesis. Heparin-binding polypeptides such as vascular endothelial growth factor (VEGF) and basic fibroblast growth factor (bFGF) are highly mitogenic for vascular endothelial cells, and are among the most potent inducers of angiogenesis. bFGF has been extracted from the subendothelial ECM produced in vitro, and from basement membranes of cornea, suggesting that ECM may serve as a reservoir for bFGF. Immunohistochemical staining revealed the localization of bFGF in basement membranes of diverse tissues and blood vessels. bFGF binds to HSPG in the ECM and can be released in an active form by HS-degrading enzymes. Heparanase expressed by platelets, mast cells, neutrophils, and lymphoma cells was found to be involved in the release of active bFGF from ECM and basement membranes, suggesting that heparanase activity may not only function in cell migration and invasion, but may also elicit an indirect neovascular response (Elkin et al., 2001).

Heparanase catalytic activity correlates with the ability of activated cells of the immune system to leave the circulation and elicit both inflammatory and autoimmune responses. Interaction of platelets, granulocytes, T and B lymphocytes, macrophages, and mast cells with the subendothelial ECM is associated with degradation of HS by heparanase (Vlodavsky et al., 1992). The enzyme is released from intracellular compartments (e.g., lysosomes, specific granules) in response to various activation signals (e.g., thrombin, calcium ionophore, immune complexes, antigens, mitogens), suggesting its regulated involvement in inflammatory sites and in autoimmune diseases. Indeed, treatment of experimental animals with heparanase substrates (e.g., non-anticoagulant species of low molecular weight heparin) markedly reduced the incidence of experimental autoimmune encephalomyelitis (EAE), adjuvant arthritis and graft rejection, indicating that heparanase inhibitors may inhibit autoimmune and inflammatory diseases (Lider et al., 1989).

Heparanase inhibitors have been proposed for treatment of human metastasis, for example, derivatives of siastatin B (Nishimura et al., 1994; Kawase et al., 1995), fungal metabolites such as derivatives isolated from the fungal strain Acremonium sp. MT70646 (WO 01/46385; Ko et al., 2000) and trachyspic acid (Shiozawa et al., 1995); heterocyclic compounds such as phthalimide carboxylic acid derivatives (WO 03/74516; Courtney et al., 2004), benzoxazole, benzthiazole and benzimidazole derivatives (WO 04/0466122; WO 04/046123) and furanthiazole derivatives (WO 04/013132); tetronic acid derivatives (Ishida et al., 2004); suramin, a polysulfonated naphthylurea (Nakajima et al., 1991), sulfated oligosaccharides, e.g., sulfated maltotetraose and maltohexaose (Parish et al., 1999), and sulfated polysaccharides (Parish et al., 1987; Lapierre et al., 1996); sulfated linked cyclitols (Freeman et al., 2005); and low molecular-weight glycol-split heparins (Naggi et al., 2005).

Heparanase inhibitors of different chemical structures have been described in the International PCT Applications WO 02/060373, WO 02/060374, WO 02/060375, and WO 02/060867, of the same applicants. Recently, the development of heparanase inhibitors has been reviewed (Ferro et al., 2004).

U.S. Pat. No. 5,968,822 discloses a polynucleotide encoding a polypeptide having heparanase catalytic activity and host cells, particularly insect cells, expressing said polypeptide. The recombinant polypeptide having heparanase activity is said to be useful for potential treatment of several diseases and disorders such as wound healing, angiogenesis, restenosis, inflammation and neurodegenerative diseases as well as for development of new drugs that inhibit tumor cell metastasis, inflammation and autoimmunity. International Patent Publication No. WO 99/57244 of the present applicants discloses bacterial, yeast and animal cells and methods for overexpressing recombinant heparanase in cellular systems. U.S. Pat. No. 6,190,875, assigned to the present applicants, discloses methods of screening agents inhibiting heparanase catalytic activity and hence potentially inhibiting tumor metastasis, autoimmune and inflammatory diseases which comprises interacting a native or recombinant heparanase enzyme with a heparin substrate in the presence or absence of an agent and determining the inhibitory effect of said agent on the catalytic activity of said heparanase enzyme towards said heparin substrate. Both U.S. 5,968,822 and U.S. 6,190,875 and further WO 99/57244 are herein incorporated by reference in their entirety as if fully disclosed herein.

WO 01/44172 discloses salicylamide compounds said to inhibit serine proteases, Urokinase (uPA), Factor Xa (Fxa), and/or Factor VIIa (FVIIa), and to have utility as anticancer agents and/or as anticoagulants for the treatment or prevention of thromboembolic disorders in mammals. WO 01/01981 and WO 01/02344 disclose certain aminobenzoic acid derivatives useful as VEGF receptor antagonists, in particular in the treatment of diseases in which VEGF is involved. Japanese Patent Publications Nos. JP 06-016597, JP 06-016601, JP 05-301849 and JP 05-271156 disclose certain 1-alkoxy-2,6-diphenoxybenzene derivatives said to exhibit antineoplastic activity. The heparanase inhibitors of the present invention have not been disclosed nor suggested in said publications.

### SUMMARY OF THE INVENTION

The present invention provides, in one aspect, a pharmaceutical composition comprising a pharmaceutically acceptable carrier and at least one heparanase inhibitor selected from compounds of the general formula I, II, III or IV hereinafter or a pharmaceutically acceptable salt thereof.

The pharmaceutical composition of the invention is particularly useful for the treatment of diseases and disorders caused by or associated with heparanase catalytic activity such as, but not limited to, cancer, inflammatory disorders and autoimmune diseases.

In another aspect, the present invention relates to the use of a heparanase inhibitor of the general formula I, II, III or IV for the manufacture of a pharmaceutical composition for the treatment of diseases and disorders caused by or associated with heparanase catalytic activity such as cancer, inflammatory disorders and autoimmune diseases.

In a further aspect, the present invention provides novel derivatives of the general formula I, II, III or IV.

In still another aspect, the present invention relates to a method for treatment of a patient suffering from a disease or disorder caused by or associated with heparanase catalytic activity such as cancer, an inflammatory disorder or an autoimmune disease, which comprises administering to said patient an amount of a heparanase inhibitor selected from the group consisting of compounds of the general formula I, II, III and IV, effective to treat said disease or disorder in said patient.

### DETAILED DESCRIPTION OF THE INVENTION

According to the present invention, pharmaceutical compositions are provided for treatment of diseases and disorders caused by or associated with heparanase catalytic activity, said compositions comprising a pharmaceutically acceptable carrier and at least one heparanase inhibitor of the general formula I, II, III or IV: 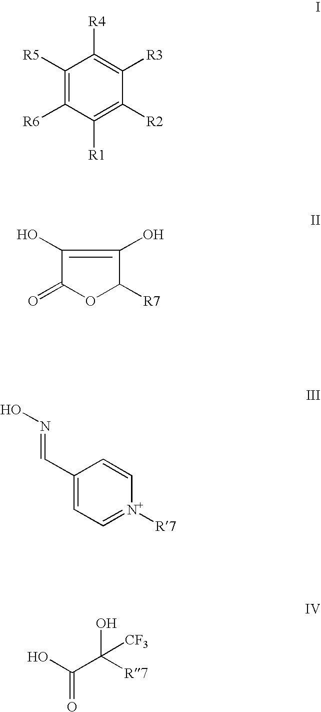
wherein

R1 is selected from the group consisting of: 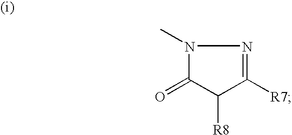
or the tautomer

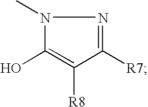

(ii) —N(R9)—CO(R10);

(iii) —CO—N(R9)(R10);

(iv) —SO₂R11;

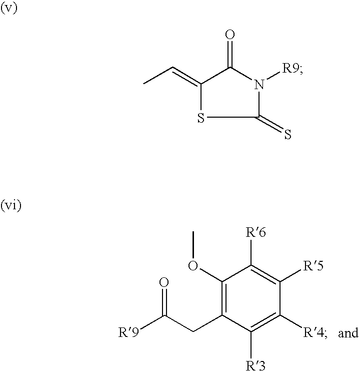

(vii) —CH(OH)—CH(NH—CO—R′7)—CH₂—NR9R′9

R2, R3, R4, R5, R6, R′3, R′4, R′5 and R′6 each independently represents hydrogen, halogen, nitro, (C1-C32) alkyl, (C2-C32) alkenyl, (C6-C14) aryl, heteroaryl, —OR′9, —SR′9, —NR9R′9, —(CH₂)ₙ—NR9-COR′9, —COR′9, —COOR′9, —(CH₂)ₙ—CO—N(R9)(R′9); —SO₃R′9, —SO₂R′9, or —NHSO₂R′9;

or R1 and R2 together are a moiety selected from the group consisting of:

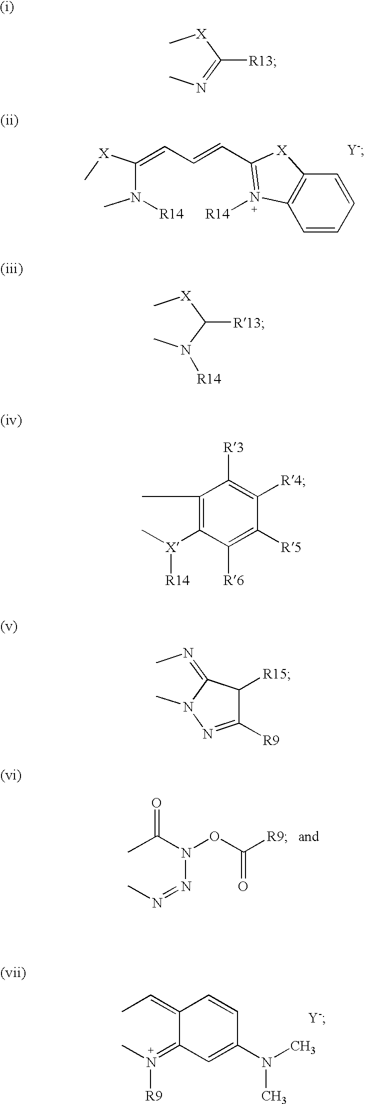

wherein X is O, S, N(R12) or C(R12′, R″12) and X′ is O or N;

or each pair of R2+R3, R3+R4, R4+R5 or R5+R6, together with the carbon atoms to which they are attached, form a 5- or 6-membered aromatic ring;

R7 is selected from the group consisting of H, halogen, (C1-C32) alkyl, (C2-C32) alkenyl, (C6-C14) aryl, heteroaryl, —OR′9, —SR′9, —NR9R′9, —NR9—COR′9, —COR′9, —COOR′9, —CH(OH)—(CH₂)ₙ—O—CO—R9, —(CH₂)ₙ—NR9-COR′9, —(CH₂)ₙ—CO—N(R9)(R′9), —SO₃R′9, —SO₂R′9, —NHSO₂R′9, —N═N—(C6-C14) aryl, and

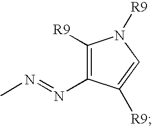

R′7 is (C1-C32) alkyl;

R″7 is (C2-C32) alkenyl;

R8 is as defined for R7;

R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms;

R10 is selected from the group consisting of (C1-C32) alkyl, (C2-C32) alkenyl, 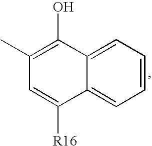
—(CH₂)ₙ—CO—R17 and —(CH₂)ₙ—NH—CO—R9-O—R′9:

R11 is OH or

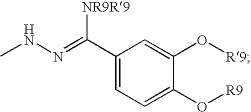

R12, R′12 and R″12 each is H or (C1-C32) alkyl, or R′12 and R″12 together are a radical

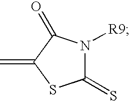

R13 is selected from the group consisting of (C1-C32) alkyl, (C6-C14) aryl, —N═CH—(C6-C14) aryl,

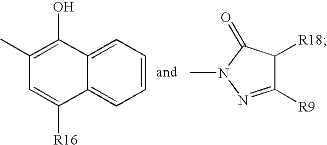

R′13 is ═O, ═NH OR ═N—NH—SO2R′9;

R14 is H, (C1-C32) alkyl, —(CH₂)ₘ—CH(OH)—CH₂—NR9R′9 or —(CH₂)ₘ—CH(OH)—(C6-C14) aryl;

R15 is H or —SO₃H;

R16 is selected from the group consisting of H, halogen, —COOH, —SO₃H, —N═N—(C6-C14) aryl and

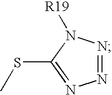

R17 is selected from the groups consisting of (C1-C32) alkyl, (C6-C14) aryl, —NH—NH—CO—(C1-C32) alkyl, —NH—NH—CO—(C6-C14) aryl, —(CH₂)ₙ—NH—CO—C(R9)-O(C1-C32) alkyl, —(CH₂)ₙ—NH—CO—C(R9)-O(C6-C14) aryl, —(CH₂)ₙ—CO—(C1-C32) alkyl and —(CH₂)ₙ—CO—(C6-C14) aryl;

R18 is H or ═N—(C6-C14) aryl;

R19 is (C6-C14) aryl;

Y⁻ is a counter ion such as chloride, bromide, iodide, perchlorate, tosylate, mesylate, sulfate, phosphate or an organic anion;

n is 0 or an integer from 1 to 10; m is an integer from 1 to 10;

any “(C1-C32) alkyl” or “(C2-C32) alkenyl” may be straight or branched and may be interrupted by one or more heteroatoms selected from the group consisting of O, S and N, and/or may be unsubstituted or substituted by one or more radicals selected from the group consisting of halogen, (C3-C7) cycloalkyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9;

“heteroaryl” means a radical derived from a mono- or poly-cyclic heteroaromatic ring containing 1 to 3 heteroatoms selected from the group consisting of O, S and N; and

any “aryl” or “heteroaryl” may be substituted by one or more radicals selected from the group consisting of halogen, (C6-C14) aryl, (C1-C32) alkyl, nitro, —OR′9, —SR′9, —COR′9, —COOR′9, —SO₃R′9, —SO₂R′9, —NHSO₂R′9, NR9R′9, —(CH₂)ₙ—NR9-COR′9, and —(CH₂)ₙ—CO—NR9R′9;

and pharmaceutically acceptable salts thereof.

As used herein the term “(C1-C32) alkyl” typically refers to a straight or branched alkyl radical having 1-32 carbon atoms and includes for example methyl, ethyl, n-propyl, isopropyl, n-butyl, isobutyl, tert-butyl, n-heptyl, 2,2-dimethylpropyl, n-hexyl, and preferably has 10 carbon atoms or more, preferably —C₁₀H₂₁, —C₁₅H₃₁, —C₁₆H₃₃, —C₁₇H₃₅, —C₁₈H₃₇, —C₂₀H₄₁ and the like.

The term “(C2-C32) alkenyl” refers to a straight or branched hydrocarbon radical having 2-32 carbon atoms and one or more double bonds, preferably a terminal double bond, and includes for example vinyl, prop-2-en-1-yl, but-3-en-1-yl, pent-4-en-1-yl, hex-5-en-1-yl, —C₁₆H₃₁ with a terminal double bond, and a group —C═C—C═.

The term “(C1-C32) alkoxy” refers to the group (C1-C32) alkyl-O—, wherein (C1-C32) alkyl is as defined above. Examples of alkoxy are methoxy, ethoxy, hexoxy, —OC₁₅H₃₁, —OC₁₆H₃₃, —OC₁₇H₃₅, —OC₁₈H₃₇, and the like.

The term “(C6-C14) aryl” refers to an aromatic carbocyclic group having 6 to 14 carbon atoms consisting of a single ring or multiple condensed rings such as phenyl, naphthyl, carbazolyl and phenanthryl optionally substituted as defined herein.

The term “heteroaryl” refers to a radical derived from a mono- or polycyclic heteroaromatic ring containing one to three heteroatoms selected from the group consisting of N, O and S. Particular examples are pyridyl, pyrrolyl, furyl, thienyl, imidazolyl, oxazolyl, quinolinyl, thiazolyl, pyrazolyl, pyrimidinyl, 1,3,4-triazinyl, 1,2,3-triazinyl, benzofuryl, isobenzofuryl, indolyl, imidazo[1,2-a]pyridyl, benzimidazolyl, benzthiazolyl and benzoxazolyl. It is to be understood that when a polycyclic heteroaromatic ring is substituted, the substitutions may be in any of the carbocyclic and/or heterocyclic rings.

The term “halogen” refers to fluoro, chloro, bromo or iodo.

In one embodiment of the present invention, the pharmaceutical composition comprises a compound of the formula Ia or I′a: 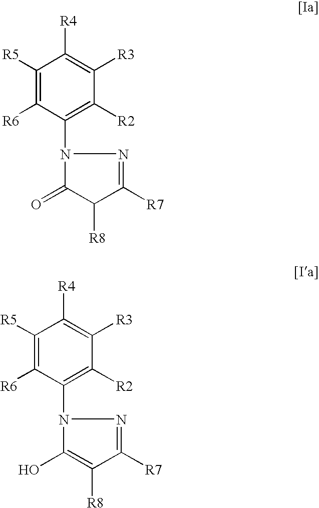
wherein

R2 is H, halogen, —NH₂ or —SO₃H;

R3 is H or —SO₃H;

R4 is H, halogen, —SO₃H, —SO₂—(C10-C22) alkyl , —O(C6-C14) aryl, or —O(C6-C14) aryl substituted by —O(C1-C8) alkyl;

R5 is H; R6 is H or halogen;

R7 is selected from the group consisting of:
(i) H;
(ii) (C10-C22) alkyl;
(iii) —COOH;
(iv) —NR9-COR′9, wherein R9 is H and R′9 is selected from the group consisting of (C10-C22) alkyl optionally substituted by epoxy, (C10-C22) alkenyl optionally substituted by —COOH, and (C6-C14) aryl optionally substituted by —SO₃H or —NH—CO—(C10-C22) alkyl; and
(v) (C6-C14) aryl optionally substituted by —SO₃H or by —NR9-COR′9, wherein R9 is H and R′9 is (C10-C22) alkyl;

R8 is selected from the group consisting of:
(i) H;
(ii) halogen;
(iii) (C2-C6) alkyl;
(iv) —O(C10-C22) alkyl;
(v) (C6-C14) aryl optionally substituted by one or more halogen, —OR′9, —COOR′9, —SO₃R′9, —NR9R′9 or —NR9COR′9, wherein R9 and R′9 each independently is H or (C10-C22) alkyl; 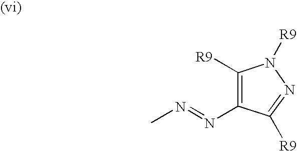
wherein R9 each independently is H or (C1-C12) alkyl; and
(vii) —N═N—(C6-C14) aryl optionally substituted by one or more halogen, —OR′9, —COOR′9, —SO₃R′9, —NHSO₂R′9, —NR9R′9, or —NR9-CO—R′9, wherein R9 and R′9 each independently is H or (C1-C6) alkyl, or R′9 is —(C6-C14) aryl substituted by methyl;

wherein any “(C10-C22) alkyl” as defined in R4, R7 and R8 may be straight or branched and may be interrupted by one or more heteroatoms selected from the group consisting of O, S and N, and/or may be substituted by one or more radicals selected from the group consisting of halogen, —(C3-C7) cycloalkyl preferably cyclopropyl, —(C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In one preferred embodiment of the present invention, the pharmaceutical composition comprises a compound of formula Ia or I′a, wherein

R2 is H, Cl, —NH₂, or —SO₃H;

R3 is H or —SO₃H;

R4 is H, Cl, —SO₃H, —SO₂C₁₆H₃₃ or phenoxy optionally substituted by ethoxy;

R5 is H, —COOH or —SO₃H;

R6 is H or Cl;

R7 is selected from the group consisting of:
(i) H;
(ii) (C17-C20) alkyl;
(iii) —COOH;
(iv) —NR9-COR′9, wherein R9 is H and R′9 is selected from the group consisting of (C11-C20) alkyl optionally substituted by epoxy, (C16-C20) alkenyl, optionally substituted by —COOH, and phenyl optionally substituted by —SO₃H or —NH—CO—C₁₇H₃₅; and
(v) phenyl, optionally substituted by —SO₃H, or by —NR9-COR′9, wherein R9 is H and R′9 is (C17-C20) alkyl; and

R8 is selected from the group consisting of:
(i) H;
(ii) Br;
(iii) isopropyl;
(iv) —OC₁₆H₃₃;
(v) phenyl optionally substituted by one or more halogen, —OR′9, —COOR′9, —SO₃R′9, —NR9R′9 or —NR9COR′9, wherein R9 and R′9 each independently is H or —C₁₆H₃₃; 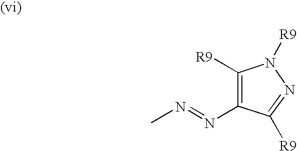
wherein R9 each independently is H, methyl or decenyl; and
(vii) —N═N-phenyl optionally substituted by one or more Cl, —OR′9, —COOR′9, —SO₃R′9, —NHSO₂R′9, —NR9R′9, or —NR9-CO—R′9, wherein R9 and R′9 each independently is H, methyl or ethyl, or R′9 is phenyl substituted by methyl.

In one preferred embodiment, the pharmaceutical composition comprises a compound of formula Ia selected from the compounds herein designated Compounds Nos. 1, 5-22, 24-30, 54, 56, 69, 71, 83, 84, 85 and 100.

In another preferred embodiment, the pharmaceutical composition comprises the compound of formula I′a herein designated Compound No. 32.

In another embodiment of the present invention, the pharmaceutical composition comprises a compound of the formula Ib:

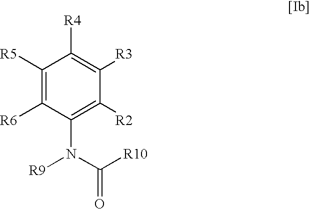

wherein

R2 is selected from the group consisting of:
(i) H;
(ii) halogen;
(iii) —OH;
(iv) —O(C10-C22) alkyl;
(v) —COOH;
(vi) —NR9R′9, wherein R9 and R′9 each independently is H, or R9 is (C1-C6) alkyl and R′9 is H or (C10-C22) alkyl; and
(vii) —O(C6-C14) aryl optionally substituted by one or more —COOH or —CO—NH₂;

R3 is H or —COOH;

R4 is selected from the group consisting of:
(i) H;
(ii) —SO₃H (iii) —O(C6-C14) aryl optionally substituted by one or more —COOH;
(iv) —S(C6-C14) aryl optionally substituted by one or more —COOH; and
(v) —NR9-CO—R′9, wherein R9 and R′9 each independently is H or (C10-C22) alkyl;

R5 is H, —COOH, —SO₃H, or —NHSO₂(C6-C14) aryl optionally substituted by one or more —COOH;

R6 is H;

R9 is H or (C10-C22) alkyl;

R10 is selected from the group consisting of:
(i) (C10-C22) alkyl optionally substituted by one or more radicals selected from the group consisting of halogen, OH, epoxy and epithio; 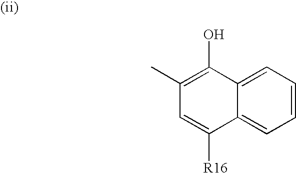
wherein
R16 is selected from the group consisting of H, halogen, —COOH, —SO₃H, S-tetrazol-5-yl optionally substituted by phenyl, and —N═N—(C6-C14) aryl optionally substituted by one or more radicals selected from the group consisting of halogen, (C1-C6) alkyl, (C6-C14) aryl, —OH, —COOH, —COOR′9, —OR′9 and —NHSO₂R′9, wherein R′9 is (C1-C6) alkyl, or phenyl optionally substituted by (C1-C6) alkyl;
(iii) —CH₂—CO—R17, wherein R17 is selected from the group consisting of (C10-C22) alkyl; (C6-C14) aryl optionally substituted by —O—(C10-C22) alkyl or by —NH—CO—(C10-C22) alkyl; and —NH—NH—CO—(C10-C22) alkyl;
(iv) —NH—(C10-C22) alkyl; and
(v) (C10-C22) alkenyl optionally substituted by oxo;

wherein any “(C10-C22) alkyl” as defined in R2, R4, R9 and R10 and the “(C10-C22) alkenyl” as defined in R10 may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═—N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10;

and wherein any “(C6-C14) aryl” as defined in R10 may be substituted by one or more radicals selected from the group consisting of halogen, (C6-C14) aryl, (C1-C32) alkyl, nitro, —OR′9, —SR′9, —COR′9, —COOR′9, —SO₃R′9, —SO₂R′9, —NHSO₂R′9, —NR9R′9, —(CH₂)ₙ—NR9-COR′9, and —(CH₂)ₙ—CO—NR9R′9.

In a preferred embodiment of the present invention, the pharmaceutical composition comprises a compound of formula Ib, wherein:

R2 is selected from the group consisting of:
(i) H;
(ii) Cl;
(iii) —OH;
(iv) —OC₁₈H₃₇;
(v) —COOH;
(vi) —NR9R′9, wherein R9 is H or methyl and R′9 is —C₁₈H₃₇; and
(vii) phenoxy optionally substituted by one or more —COOH or —CO—NH₂;

R3 is H or —COOH;

R4 is selected from the group consisting of:
(i) H;
(ii) —SO₃H
(iii) phenoxy optionally substituted by one or more —COOH;
(iv) phenylthio optionally substituted by one or more —COOH; and
(v) —NR9-CO—R′9, wherein R9 and R′9 each independently is H or —C₁₇H₃₅;

R5 is H, —COOH, —SO₃H, or —NHSO₂-phenyl optionally substituted by one or more —COOH;

R6 is H;

R9 is H or —C₁₈H₃₇;

R10 is selected from the group consisting of:
(i) —C₁₇H₃₅, optionally substituted by one or more radicals selected from the group consisting of Cl, OH, epoxy and epithio; 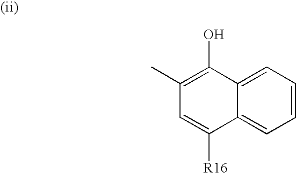
wherein
R16 is selected from the group consisting of H, Br, —COOH, —SO₃H, S-tetrazol-5-yl optionally substituted by phenyl, and —N═N-phenyl optionally substituted by one or more radicals selected from the group consisting of Cl, methyl, phenyl, —OH, —COOH, —COOR′9, —OR′9 and —NHSO₂R′9, wherein R′9 is methyl, or phenyl optionally substituted by methyl;
(iii) —CH₂—CO—R17, wherein R17 is selected from the group consisting of —C₁₇H₃₅ or —C₁₈H₃₅; phenyl, optionally substituted by —OC₁₈H₃₇ or by —NH—CO—(C15-C20) alkyl, preferably —C₁₇H₃₅; and —NH—NH—CO—(C15-C20) alkyl, preferably —C₁₇H₃₅;
(iv) —NH—C₁₈H₃₇; and
(v) (C16-C20) alkenyl, preferably —C₁₇H₃₃ and —C₁₆H₃₁, optionally substituted by oxo.

In one preferred embodiment, the pharmaceutical composition comprises a compound of formula Ib, wherein R10 is —C₁₇H₃₅, selected from the group of compounds herein designated Compounds Nos. 61, 87, 92, 93, 95 and 96.

In another preferred embodiment, the pharmaceutical composition comprises a compound of formula Ib, wherein R10 is 1-hydroxy-4-R18-2-naphthyl, selected from group of compounds herein designated Compounds Nos. 3, 33, 34, 40, 41, 43, 45, 46, 47, 49, 50, 52, 53, 55, 62, 63 and 77.

In a further preferred embodiment, the pharmaceutical composition comprises a compound of formula Ib, wherein R10 is —CH₂—CO—R17, selected from the group of compounds herein designated Compounds Nos. 2, 23, 44, 51, 60 and 64.

In still a further preferred embodiment, the pharmaceutical composition comprises the compound of formula Ib herein designated Compound No. 70, wherein R10 is —NH—C₁₈H₃₇.

In yet still a further preferred embodiment, the pharmaceutical composition comprises a compound of formula Ib wherein R10 is —(C10-C22) alkenyl, selected from the compounds herein designated Compounds Nos. 86 and 94.

In a further embodiment of the present invention, the pharmaceutical composition comprises a compound of the formula Ic: 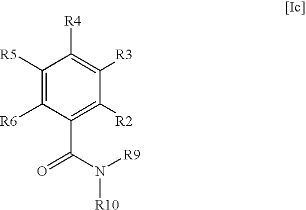
wherein

R2, R3, R4, R5, and R6 each independently represents hydrogen, halogen, nitro, (C1-C32) alkyl, (C2-C32) alkenyl, (C6-C14) aryl, heteroaryl, OR9′, —SR9′, —NR9R′9, —(CH₂)ₙ—NR9-COR′9, —COR′9, —COOR′9, —(CH₂)ₙ—CO—N(R9)(R′9); —SO₃R′9, —SO₂R′9, or —NHSO₂R′9;

or R3 and R4 together with the carbon atoms to which they are attached form a condensed benzene ring;

R9 is H or (C1-C32) alkyl and R′9 is H, (C1-C32) alkyl, (C2-C32) alkenyl or (C6-C14) aryl, or R9 and R′9 as part of the radical —NR9R′9 form together with the N atom to which they are attached a 3-7 membered saturated ring, optionally further containing one or more N, S or O atoms;

R10 is
(i) (C10-C22) alkyl; or
(ii) —(CH₂)ₙ—NH—CO—R9-O—O′9, wherein R9 is (C1-C6) alkyl, R′9 is (C6-C14) aryl substituted by —C₁₅H₃₁; and n is an integer of 1 to 6;

and wherein the “(C1-C32) alkyl” and “(C2-C32) alkenyl” as defined in R2 to R6 and R9 and the “(C10-C22) alkyl” as defined in R10 may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In a preferred embodiment of the present invention, the pharmaceutical composition comprises a compound of formula Ic, wherein R2 is OH; R3 and R4 together with the carbon atoms to which they are attached form a condensed benzene ring; R5 is H or —SO₃H; R6 and R9 each is H; and R10 is (i) —C₁₈H₃₇; or (ii) —(CH₂)ₙ—NH—CO—R9-O—O—R′9, wherein R9 is —CH(C₂H₅), R′9 is phenyl substituted by —C₁₅H₃₁; and n is 3.

In one preferred embodiment, the pharmaceutical composition comprises a compound of formula Ic selected from the compounds herein designated Compound Nos. 31 and 72.

In still another embodiment of the present invention, the pharmaceutical composition comprises a compound of the formula Id:

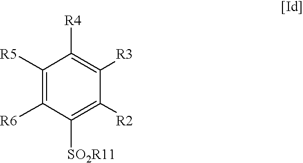

wherein

R2 is H;

R3 is H, —COOH, —NH₂ or 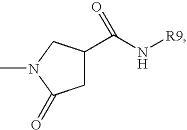
wherein R9 is (C10-C22) alkyl;

R4 is selected from the group consisting of:
(i) H;
(ii) —O—(C10-C22) alkyl;
(iii) —NH—(C10-C22) alkyl;
(iv) —SO₂—(C10-C22) alkyl; 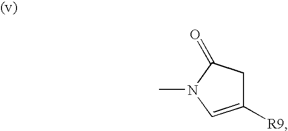
wherein R9 is (C10-C22) alkyl; and
(vi) phenoxy, optionally substituted by at least one substituent selected from —SO₃H or 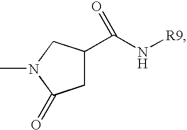
wherein R9 is (C10-C22) alkyl;

R5 is H, —COOH, or —NH₂;

R6 is H or phenoxy optionally substituted by halogen, —COOH or —CO—NH₂;

R11 is OH or

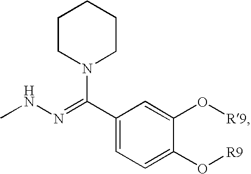

wherein R9 is (C10-C22) alkyl and R′9 is (C1-C6) alkyl; and

wherein any “(C10-C22) alkyl” as defined in R4 and R9 may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or —(C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In a preferred embodiment of the present invention, the pharmaceutical composition comprises a compound of the formula Id, wherein

R2is H;

R3 is H, —COOH, —NH₂, or 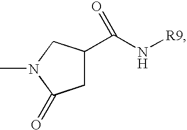
wherein R9 is —C₁₈H₃₇;

R4 is selected from the group consisting of:
(i) H;
(ii) —O—C₁₆H₃₃;
(iii) —NH—C₁₉H₃₉;
(iv) —SO₂—C₁₆H₃₃; 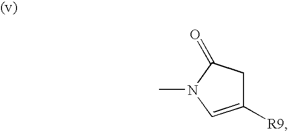
wherein R9 is —C₁₅H₃₁; and
(vi) phenoxy, optionally substituted by at least one substituent selected from —SO₃H and 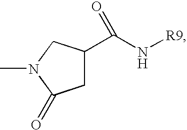
wherein R9 is —C₁₈H₃₇;

R5 is H, —COOH, or —NH₂;

R6 is H or phenoxy optionally substituted by halogen, —COOH or —CO—NH₂;

R11 is OH or

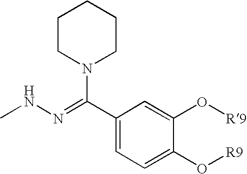

wherein R9 is —C₁₆H₃₃, and R′9 is methyl.

In a preferred embodiment, the pharmaceutical composition comprises a compound of the formula Id selected from the compounds herein designated Compounds Nos. 75, 76, 88, 89, 101, 103, 104, 105, 106 and 107.

In still a further embodiment of the present invention, the pharmaceutical composition comprises a compound of the formula Ie:

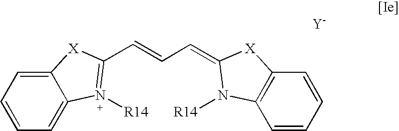

wherein

X is O or S; and

R14 is (C10-C22) alkyl;

Y⁻ is a counter ion selected from the group consisting of chloride, bromide, iodide, perchlorate, tosylate, mesylate, sulfate, phosphate and an organic anion;

and wherein the “(C10-C22) alkyl” as defined in R14 may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or —(C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In one preferred embodiment, the pharmaceutical composition comprises a compound of the formula Ie, wherein X is O or S, R14 is —C₁₈H₃₇; and Y⁻ is perchlorate.

In another preferred embodiment, the pharmaceutical composition comprises a compound of the formula Ie selected from the compounds herein designated Compounds Nos. 66 and 67.

In yet another embodiment of the present invention, the pharmaceutical composition comprises a compound of the formula If:

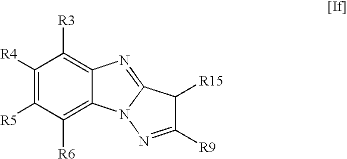

wherein

R3 and R5 each is H;

R4 is H, —COOH or —SO₃H;

R6 is H or —COOH;

R9 is H or (C10-C22) alkyl; and

R15 is H or —SO₃H;

and wherein the “(C10-C22) alkyl” as defined in R9 may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In a preferred embodiment of the present invention, the pharmaceutical composition comprises a compound of the formula If, wherein R3 and R5 are H; R6 is H or —COOH; R4 is selected from the group consisting of H, —COOH and —SO₃H; R9 is H or —C₁₇H₃₅; and R15 is H or —SO₃H.

In a more preferred embodiment, the pharmaceutical composition comprises a compound of the formula If selected from the compounds herein designated Compounds Nos. 4, 35 and 36.

In yet a further embodiment of the present invention, the pharmaceutical composition comprises a compound of the formula Ig:

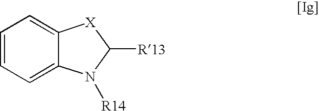

wherein

X is NR12 or CR′12R″12;

R12 is (C10-C22) alkyl;

R′12 and R″12 each is (C1-C6) alkyl, or R′12 and R″12 together are a radical

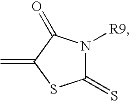

wherein R9 is H or (C10-C22) alkyl substituted by —COOH;

R′13 is ═O, ═NH or ═N—NH—SO₂—(C6-C14) aryl, wherein the aryl is either substituted by —COOH and —O—(C10-C22) alkyl, or by —NH—SO₂-phenyl, wherein the phenyl is substituted by —COOH and —O—(C10-C22) alkyl; and

R14 is (C1-C8) alkyl or —CH₂—CH(OH)—(C6-C14) aryl substituted by one or more (C1-C6) alkoxy;

wherein any “(C10-C22) alkyl” as defined in R12 and R′13 may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, (C6-C14) aryl, nitro, —OR′9, —SR′9, epoxy, epithio, oxo —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In a preferred embodiment of the present invention, the pharmaceutical composition comprises a compound of the formula Ig, wherein

X is NR12 or CR′12R″12;

R12 is —C₁₆H₃₃;

R′12 and R″12 each is methyl, or R′12 and R″12 together are a radical

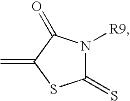

wherein R9 is H or —C₁₀H₂₀—COOH;

R′13 is ═O, ═NH or ═N—NH—SO₂-phenyl, wherein the phenyl is either substituted by —COOH and —OC₁₈H₃₇ or by —NH—SO₂-phenyl, wherein the phenyl is substituted by —COOH and —OC₁₈H₃₇; and

R14 is methyl, ethyl, or —CH₂—CH(OH)-phenyl substituted by one or more methoxy groups.

In a preferred embodiment, the pharmaceutical composition comprises a compound of the formula Ig selected from the compounds herein designated Compounds Nos. 48, 59 65 and 82.

In still a further embodiment of the present invention, the pharmaceutical composition comprises a compound of the formula Ih:

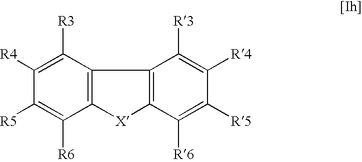

wherein

X′ is O or NR14;

R3, R4, R5, R′3 and R′5 each is H or halogen;

R′4 is H, halogen or (C10-C22) alkenyl;

R6 and R′6 each is H or —COOH; and

R14 is (C10-C22) alkyl interrupted by one or more N atoms and substituted by hydroxy;

and wherein the “(C10-C22) alkenyl” as defined in R′4 may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is H, (C1-C32) alkyl, (C2-C32) alkenyl or (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In a preferred embodiment of the present invention, the pharmaceutical composition comprises a compound of the formula Ih, wherein

X′ is O or NR14;

R3, R4, R5, R′3 and R′5 each is H, Cl or Br;

R′4 is H, Cl, Br or —C₂₀H₃₉;

R6 and R′6 each is H or —COOH; and

R14 is —C₁₀H₂₁—NH—CH₂—CH(OH)—CH₂— or —C₁₈H₃₇—NH—CH₂—CH(OH)—CH₂—.

In a preferred embodiment, the pharmaceutical composition comprises a compound of the formula Ih selected from the compounds herein designated Compounds Nos. 68, 90 and 91.

In still a further embodiment of the present invention, the pharmaceutical composition comprises a compound of the formula Ii:

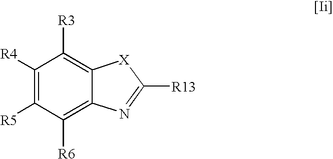

wherein

X is O, S or NR12;

R3 is H or —COOH;

R4 is H or —SO₃H;

R5 is H, —COOH or —SO₃H;

R6is H;

R12 is H or (C10-C22) alkyl;

R13 is selected from the group consisting of:
(i) (C1-C6) alkyl; 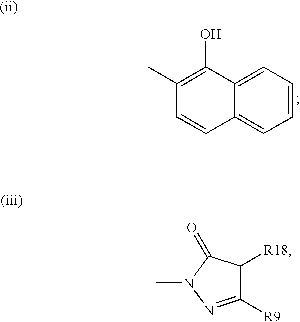
wherein R9 is (C10-C22)alkyl and R18 is H or ═N—(C6-C14) aryl
wherein the aryl is optionally substituted by —NR9R′9, wherein R9 and R′9 each is (C1-C6) alkyl;
(iv) (C6-C14) aryl optionally substituted by 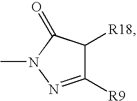
wherein R9 is (C10-C22) alkyl and R18 is ═N—(C6-C14) aryl, wherein the aryl is optionally substituted by —NR9R′9, wherein R9 and R′9 each is (C1-C6) alkyl; and
(v) —N═CH—(C6-C14) aryl substituted by —OH and by one or more halogen atoms, or by —OH or nitro, or both;

wherein any “(C10-C22) alkyl” as defined in R12 and R13 may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In a preferred embodiment of the present invention, the pharmaceutical composition comprises a compound of the formula Ii, wherein

X is O, S or NR12;

R4 is H or —SO₃H;

R6 is H;

R3 is H or —COOH;

R5 is H, —COOH or —SO₃H;

R12 is H, —C₁₆H₃₃ or —C₁₈H₃₇;

R13 is selected from the group consisting of:
(i) methyl; 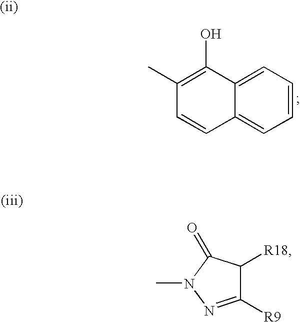
wherein R9 is —C₁₇H₃₅ and R18 is H or ═N-phenyl, wherein the phenyl is optionally substituted by —NR9R′9, wherein R9 and R′9 each is ethyl;
(iv) phenyl, optionally substituted by 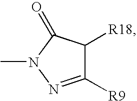
wherein R9 is —C₁₇H₃₅ and R18 is ═N-phenyl, wherein the phenyl is optionally substituted by —NR9R′9, wherein R9 and R′9 each is ethyl; and
(v) —N═CH-phenyl, optionally substituted by —OH and one or more Cl or Br, or naphthyl optionally substituted by —OH or nitro, or both.

In a preferred embodiment, the pharmaceutical composition comprises a compound of the formula Ii selected from the compounds herein designated Compounds Nos. 37, 38, 39, 42, 57, 58, 73 and 102.

In still a further embodiment of the present invention, the pharmaceutical composition comprises a compound of the formula Ij:

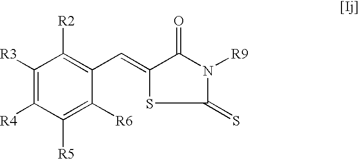

wherein

R2, R4, R5 and R6 each is H;

R3 is H or halogen; and

R9 is H or (C10-C22) alkyl substituted by —COOH;

In a preferred embodiment, the pharmaceutical composition comprises a compound of the formula Ij, wherein R2, R4, R5 and R6 each is H; R3 is H or Br; and R9 is H or —C₁₀H₂₀—COOH, more preferably the compound herein designated Compound No. 81.

In still a further embodiment of the present invention, the pharmaceutical composition comprises a compound of the formula Ik:

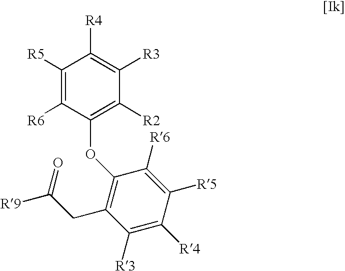

wherein

R2, R4, R6, R′3, R′5 and R′6 each is H;

R3, R5 and R′4 each is H or —COOH; and

R′9 is (C10-C22) alkenyl optionally substituted by OH and —CF₃; and wherein the “(C10-C22) alkenyl” as defined in R′9 may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, —(C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO2HR′9 and —PO₃R9R′9; R9 is H or (C1-C32) alkyl and R′9 is selected from the group consisting of H, (C1-C32) alkyl, (C2-C32) alkenyl and —(C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In a preferred embodiment of the present invention, the pharmaceutical composition comprises a compound of the formula Ik, wherein R2, R4, R6, R′3, R′5 and R′6 each is H; R3, R5 and R′4 each is —COOH; and R′9 is —C₁₇H₃₁ optionally substituted by OH and —CF₃, more preferably the compound herein designated Compound No. 98.

In still a further embodiment of the present invention, the pharmaceutical composition comprises a compound of the formula Il:

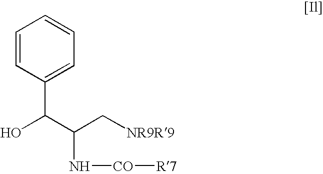

wherein

R′7 is (C10-C22) alkyl;

R9 and R′9 together with the N atom to which they are attached form a 3-7 membered saturated ring, optionally containing a further O, N or S atom;

and wherein any “(C10-C22) alkyl” as defined in R′7, may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein in this context R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In a preferred embodiment, the pharmaceutical composition comprises the compound of the formula Il, herein designated Compound No. 74, wherein R′7 is —C₁₅H₃₁ and R9 and R′9 together with the N atom to which they are attached form a morpholine ring.

In still a further embodiment of the present invention, the pharmaceutical composition comprises a compound of the formula Im:

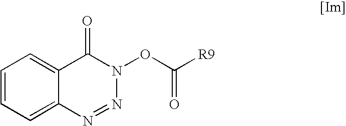

wherein

R9 is (C10-C22) alkyl that may be straight or branched and may be interrupted by one or more heteroatoms selected from the group consisting of O, S and N, and/or may be substituted by one or more radicals selected from the group consisting of halogen, —(C3-C7)cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein in this context R9 is H or —(C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In a preferred embodiment of the present invention, the pharmaceutical composition comprises a compound of the formula Im, wherein R9 is —C₁₇H₃₃ optionally substituted by epoxy, more preferably the compound herein designated Compound No. 99.

In still a further embodiment of the present invention, the pharmaceutical composition comprises a compound of the formula In:

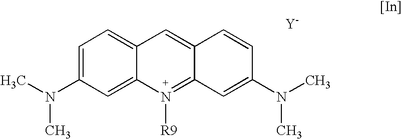

wherein

R9 is (C10-C22) alkyl; and

Y⁻ is a counter ion selected from the group consisting of chloride, bromide, iodide, perchlorate, tosylate, mesylate, sulfate, phosphate and an organic anion;

and wherein the “(C10-C22) alkyl” as defined in R9 may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO2HR′9 and —PO₃R9R′9; and wherein in this context R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In a preferred embodiment of the present invention, the pharmaceutical composition comprises a compound of the formula In, herein designated Compound No. 79, wherein R9 is —C₁₈H₃₇ and Y⁻ is bromide.

In yet still a further embodiment of the present invention the pharmaceutical composition comprises a compound of the general formula II:

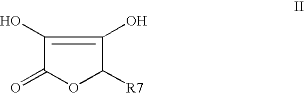

wherein

R7 is —CH(OH)—CH₂—O—CO—R9 and R9 is (C10-C22) alkyl;

and wherein the “(C10-C22) alkyl” as defined in R9 may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein in this context R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In a preferred embodiment, the pharmaceutical composition comprises the compound herein designated Compound No. 78, wherein R7 is —CH(OH)—CH₂—O—CO—R9 and R9 is —C₁₅H₃₁.

In yet still a further embodiment of the present invention, the pharmaceutical composition comprises a compound of the general formula III:

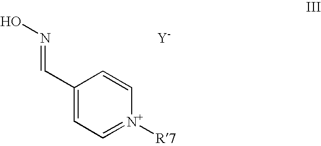

wherein

R′7 is (C10-C22) alkyl; and

Y⁻ is a counter ion selected from the group consisting of chloride, bromide, iodide, perchlorate, tosylate, mesylate, sulfate, phosphate and an organic ion;

and wherein the “(C10-C22) alkyl” as defined in R′7 may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein in this context R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In a preferred embodiment, the pharmaceutical composition comprises the compound of formula III, herein designated Compound No. 80, wherein R′7 is —C₁₆H₃₃ and Y⁻ is bromide.

In yet still a further embodiment of the present invention, the pharmaceutical composition comprises a compound of the general formula IV:

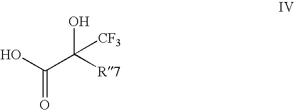

wherein R″7 is (C2-C32) alkenyl that may be straight or branched and may be interrupted by one or more heteroatoms selected from the group consisting of O, S and N, and/or may be substituted by one or more radicals selected from the group consisting of halogen, (C3-C7)cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein in this context R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

In a preferred embodiment, the pharmaceutical composition comprises the compound of formula IV, herein designated Compound No. 97, wherein R″7 is —C₁₆H₃₁.

Although we have presented the compounds of general formula I in 14 different groups Ia-In, it is obvious that many of the compounds carry functional groups or chemical characteristics that are common to more than one group Ia-In and could easily be classified in one or more of the other groups of compounds.

The compounds herein designated Compounds Nos. 12, 18, 27, 37, 48, 50, 61-63, 70, 71, 75, 77, 83-87, 90-96 and 98-107, represented by the general formula I, II, III or IV are new chemical entities and as such represent a further aspect of the present invention.

Also contemplated by the present invention are pharmaceutically acceptable salts of the compounds of formula I, II, III or IV, both salts formed by any carboxy or sulfo groups present in the molecule and a base as well as acid addition and/or base salts.

Pharmaceutically acceptable salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, ethylene-diamine, N-methylglucamine, and procaine (see, for example, Berge S. M., et al., “Pharmaceutical Salts,” (1977) J. of Pharmaceutical Science, 66:1-19). The salts can also be pharmaceutically acceptable quaternary salts such as a quaternary salt of the formula —⁺NRR′R″Z⁻, wherein R, R′ and R″ each is independently hydrogen, alkyl or benzyl and Z is a counterion, such as chloride, bromide, iodide, O-alkyl, toluenesulfonate, methylsulfonate, sulfonate, phosphate, or carboxylate.

Pharmaceutically acceptable acid addition salts of the compounds include salts derived from inorganic acids such as hydrochloric, nitric, phosphoric, sulfuric, hydrobromic, hydriodic, phosphorous, and the like, as well as salts derived from organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monohydrogenphosphate, dihydrogenphosphate, metaphosphate, pyro-phosphate, chloride, bromide, iodide, acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like and gluconate or galacturonate (see, for example, Berge S. M., et al., “Pharmaceutical Salts,” (1977) J. of Pharmaceutical Science, 66:1-19).

The acid addition salts of said basic compounds are prepared by contacting the free base form with a sufficient amount of the desired acid to produce the salt in the conventional manner. The free base form may be regenerated by contacting the salt form with a base and isolating the free base in the conventional manner. The free base forms differ from their respective salt forms somewhat in certain physical properties such as solubility in polar solvents, but otherwise the salts are equivalent to their respective free base for purposes of the present invention.

The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention.

The inhibitory effect of the compounds of the present invention on heparanase activity can be evaluated by several methods carried out in vitro, ex vivo, or in vivo.

Some of the in vitro assays used according to the present invention were described in U.S. Pat. No. 6,190,875. In these assays, heparanase is incubated with a heparanase substrate in the presence and in the absence of a compound of the present invention, and the inhibitory effect of the compound on the catalytic activity of the heparanase on its substrate is evaluated.

The heparanase may be natural mammalian heparanase, such as human heparanase purified as described in U.S. Pat. No. 5,362,641 or, preferably, recombinant mammalian, e.g. human or mouse recombinant heparanase as described in U.S. Pat. No. 5,968,822, US 6,190,875, and WO 99/57244, in purified or non-purified form. A source of non-purified recombinant heparanase is, for example, an extract of cells in which mammalian heparanase cDNA is expressed.

The heparanase substrate may be a natural heparan sulfate substrate, or an alternative substrate of the enzyme as described in U.S. Pat. No. 6,190,875, for example, heparin (e.g. heparin immobilized on a gel such as Sepharose), heparin fragments (e.g. several species of low molecular weight heparin), modified non-anticoagulant species of heparin, other sulfated polysaccharides (e.g. pentosan polysulfate), soluble HSPG or ECM.

Evaluation of the inhibitory effect can be carried out, for example, as described in U.S. Pat. No. 6,190,875, by a size separation assay adapted for detection of degradation products of the heparanase substrate. Examples of such assays include gel electrophoresis and colunm chromatography.

Qualitative and quantitative evaluation of the catalytic activity of heparanase on its substrate and the inhibitory effect of a candidate inhibitor can be effected, for example, by calorimetric assays. Any calorimetric assay based on any color producing reaction is envisaged by the invention, be it a simple color reaction, which is readily detectable, or a fluorimetric or a luminiscent (e.g., chemiluminiscent) reaction, which are readily detectable by fluorescence detecting techniques. Examples of such suitable calorimetric assays include, but are not limited to, the dimethylmethylene blue (DMB), tetrazolium blue and carbazole assays. Qualitative calorimetric assays include the dimethylmethylene blue (DMB) assay, which yields color shift in the presence of polyanionic compounds such as sulfated glycosaminoglycans having different sizes that are released from the substrate (soluble or immobilized), and the carbazole assay, which detects uronic acid derivatives present in complete hydrolyzates of products released from an immobilized substrate, both assays being applicable for crude extracts of heparanase and for the purified enzyme as well.

In a preferred embodiment, a quantitative evaluation is desired and the preferred in vitro assays are those which are adapted for detection of reducing moieties associated with degradation products of the heparanase substrate, preferably a reducing sugar assay. An example of a quantitative colorimetric assay is the tetrazolium blue assay which allows calorimetric detection of reducing moieties released from the substrate, e.g. heparan sulfate, which may be present either in soluble or immobilized form.

Another possibility, although less preferred, consists of evaluating the catalytic activity of heparanase on the substrate by radioactive techniques, in which case the substrate used is radiolabeled, either in vitro or metabolically.

The ex vivo assays for evaluating the inhibitory effect of the compounds on heparanase activity include angiogenic sprout formation and transmigration assays. The angiogenic sprout formation assay is carried out in the rat aorta model (Nicosia et al., 1997; Nicosia and Ottinetti, 1990), whereby rat aorta rings are embedded in a basement membrane-like matrix composed of ECM-derived proteins such as laminin and collagen type IV, and HSPG, thus constituting a relevant heparanase substrate. The rings then develop angiogenic sprouts and angiogenesis can be quantitated. The compounds to be tested are added to the embedded aortic rings and their effect on angiogenic sprout formation is then evaluated.

In the ex vivo transwell migration assay, immune cell migration is evaluated, optionally in the presence of a chemoattractant factor such as stromal cell-derived factor 1 (SDF-1), a process which mimics in vivo extravasation of immune cells from the vasculature to sites of inflammation. In this assay, immune cells such as lymphocytes are let to migrate from the upper to the lower chamber through a transwell filter coated with a basement membrane-like matrix composed of ECM-derived proteins. The migration rate of the cells through the filter is then evaluated by counting the number of cells migrated through the filter (e.g. using a FACS ort) compared to the number of cells added on top of the upper chamber. Overexpression of heparanase in the immune cells results in an increase in the transmigration rate of the cells while addition of a heparanase inhibitor reduces the transmigration rate of the cells.

The inhibitory effect of the compounds on heparanase activity may be also assayed in vivo, for example, using the primary tumor growth or metastasis animal models or the sponge inflammation assay.

In the primary tumor animal model, animals are injected subcutaneously (s.c.) with tumor cells and treated with the heparanase inhibitors. Tumor growth is measured when animals in untreated control group start to die. For example, primary tumors may be generated with B 16-F1 melanoma cells or with a highly metastatic subclone thereof injected s.c. into the flanks of mice. The mice are treated with heparanase inhibitors injected intraperitoneally (i.p.) twice a day starting 4 days after cell injection and are sacrificed and the tumor is measured about 3 weeks after cell injection.

In the metastasis animal model, animals are injected intravenously (i.v.) with tumor cells and treated with the heparanase inhibitors. The number of lung metastasis is counted when animals in untreated control group start to die or about 3 weeks after cell injection. For example, metastasis may be generated with B16-F1 melanoma cells or with a highly metastatic subclone thereof injected i.v. to mice. The mice are treated with heparanase inhibitors injected i.p. at certain times following cell injection, and are then sacrificed and the number of lung metastasis is counted.

In the sponge inflammation assay, polyvinyl alcohol (PVA) sponges are implanted under the mouse skin and the mouse is kept untreated or is treated with a test inhibitor agent. One day later, the mouse is sacrificed, the sponges are taken out, squeezed into a tube and the number of cells in each sample is determined. After centrifugation, the myeloperoxidase (MPO) content may be determined in a suspension of the cell pellets, and the TNF-α content in the supernatant of the sample. This assay mimics the inflammatory reaction resulting from the presence of a foreign body in the organism.

The heparanase inhibitors of the present invention can be used for the treatment of diseases and disorders caused by or associated with heparanase catalytic activity such as, but not limited to, cancer, inflammatory disorders and autoimmune diseases.

Thus, in one embodiment of the present invention, the compounds can be used for inhibition of angiogenesis, and are thus useful for the treatment of diseases and disorders associated with angiogenesis or neovascularization such as, but not limited to, tumor angiogenesis, ophthalmologic disorders such as diabetic retinipathy and macular degeneration, particularly age-related macular degeneration, reperfusion of gastric ulcer, and also for contraception or for inducing abortion at early stages of pregnancy.

In another embodiment of the invention, the compounds of general formula I, II, III or IV are useful for treatment or inhibition of a malignant cell proliferative disease or disorder.

According to this embodiment and due to the angiogenesis inhibitory activity of the compounds, they can be used for the treatment or inhibition of non-solid cancers, e.g hematopoietic malignancies such as all types of leukemia, e.g. acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma, as well as for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extrahepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

It is to be understood that the compounds of the general formula I, II, III or IV are useful for treating or inhibiting tumors at all stages, namely tumor formation, primary tumors, tumor progression or tumor metastasis.

The compounds of general formula I, II, III or IV are also useful for inhibiting or treating cell proliferative diseases or disorders such as psoriasis, hypertrophic scars, acne and sclerosis/scleroderma, and for inhibiting or treatment of other diseases or disorders such as polyps, multiple exostosis, hereditary exostosis, retrolental fibroplasia, hemangioma, and arteriovenous malformation.

In a further embodiment, the compounds of general formula I, II, III or IV are useful for treatment of or amelioration of inflammatory symptoms in any disease, condition or disorder where immune and/or inflammation suppression is beneficial such as, but not limited to, treatment of or amelioration of inflammatory symptoms in the joints, musculoskeletal and connective tissue disorders, or of inflammatory symptoms associated with hypersensitivity, allergic reactions, asthma, atherosclerosis, otitis and other otorhinolaryngological diseases, dermatitis and other skin diseases, posterior and anterior uveitis, conjunctivitis, optic neuritis, scleritis and other immune and/or inflammatory ophthalmic diseases.

In another preferred embodiment, the compounds of formula I, II, III or IV are useful for treatment of or amelioration of an autoimmune disease such as, but not limited to, Eaton-Lambert syndrome, Goodpasture's syndrome, Grave's disease, Guillain-Barré syndrome, autoimmune hemolytic anemia (AIHA), hepatitis, insulin-dependent diabetes mellitus (IDDM), systemic lupus erythematosus (SLE), multiple sclerosis (MS), myasthenia gravis, plexus disorders e.g. acute brachial neuritis, polyglandular deficiency syndrome, primary biliary cirrhosis, rheumatoid arthritis, scleroderma, thrombocytopenia, thyroiditis e.g. Hashimoto's disease, Sjögren's syndrome, allergic purpura, psoriasis, mixed connective tissue disease, polymyositis, dermatomyositis, vasculitis, polyarteritis nodosa, polymyalgia rheumatica, Wegener's granulomatosis, Reiter's syndrome, Behçet's syndrome, ankylosing spondylitis, pemphigus, bullous pemphigoid, dermatitis herpetiformis, Crohn's disease or autism.

Pharmaceutical compositions for use in accordance with the present invention may be formulated in conventional manner using one or more physiologically acceptable carriers or excipients. The carrier(s) must be acceptable in the sense that it is compatible with the other ingredients of the composition and are not deleterious to the recipient thereof.

The term “carrier” refers to a diluent, adjuvant, excipient, or any other suitable vehicle. Such pharmaceutical carriers can be sterile liquids such as water and oils.

The pharmaceutical composition can be administered systemically, for example by parenteral, e.g. intravenous, intraperitoneal or intramuscular injection. In another example, the pharmaceutical composition can be introduced to a site by any suitable route including intravenous, subcutaneous, transcutaneous, topical, intramuscular, intraarticular, subconjunctival, or mucosal, e.g. oral, intranasal, or intraocular.

In one specific embodiment, the pharmaceutical composition is administered to the area in need of treatment. This may be achieved by, for example, local infusion during surgery, topical application, direct injection into the inflamed joint, directly onto the eye, etc.

For oral administration, the pharmaceutical preparation may be in liquid form, for example, solutions, syrups or suspensions, or in solid form as tablets, capsules and the like. For administration by inhalation, the compositions are conveniently delivered in the form of drops or aerosol sprays. For administration by injection, the formulations may be presented in unit dosage form, e.g. in ampoules or in multidose containers with an added preservative.

The compositions of the invention can also be delivered in a vesicle, in particular in liposomes. In another embodiment, the compositions can be delivered in a controlled release system.

The amount of the therapeutic or pharmaceutical composition of the invention which is effective in the treatment of a particular disease, condition or disorder will depend on the nature of the disease, condition or disorder and can be determined by standard clinical techniques. In general, the dosage ranges from about 0.01 mg/kg to about 50-100 mg/kg. In addition, in vitro assays as well as in vivo experiments may optionally be employed to help identify optimal dosage ranges. The precise dose to be employed in the formulation will also depend on the route of administration, and the seriousness of the disease, condition or disorder, and should be decided according to the judgment of the practitioner and each patient's circumstances. Effective doses may be extrapolated from dose-response curves derived from in vitro or animal model test systems. For example, in order to obtain an effective mg/kg dose for humans based on data generated from mice or rat studies, the effective mg/kg dosage in mice or rats is divided by twelve or six, respectively.

The invention will now be illustrated by the following non-limiting examples.

### EXAMPLES

I. Chemical Section

The Compounds Nos. 1-107, which structural formulas are presented in Appendix A hereinafter, are identified in the description, in the examples and in the claims herein by their respective numbers in bold. The table in Appendix A presents the Chemical Abstracts Number (CAS No.) of the known compounds.

Materials:

Compounds Nos. 1-3, 28-30, 60, 66-67, 69, 72-79, 88-89 and 97 were purchased from Sigma-Aldrich (Milwaukee, Wis., USA); Compounds Nos. 4-27, 31-55, 62 and 63 were purchased from ChemStar (Moscow, Russia); Compounds Nos. 56-59 were purchased from SPECS and BioSPECS (Rijswijk, The Netherlands); Compounds Nos. 64, 65, 68, and 80-82 were purchased from Interbioscreen Ltd. (Chemogolovka, Russia); Compounds 61, 70-71, 83-87, 90-96, 98-107 were synthesized as described hereinafter.

### Example 1

### Preparation of Compound No. 61

Compound No. 61 was prepared starting from 5-(4-methoxycarbonyl-2-octadecanoylamino-phenoxy)-isophthalic acid dimethyl ester as follows:

### (i) Preparation of 5-(4-methoxycarbonyl-2-octadecanoylamino-phenoxy)-isophtlialic acid dimethyl ester

Dimethyl 5-(2-amino-4-(methoxycarbonyl)phenoxy) isophthalate (1 gr, 2.8 mmol) was dissolved in 200 ml of chloroform. Stearoyl chloride (2.6 ml, 7.6 mmol) and triethylamine (0.4 ml, 2.8 mmol) were added. The mixture was refluxed for 1 hr. The solvent was evaporated and the product was purified by chromatography using hexane:EtOAc (6:4) as eluents. The product was recrystallized from EtOH and the title compound was collected (0.75 gr, 1.2 mmol) in 43% yield. ¹H NMR (DMSO): δ 8.57 (s, 1H), 8.26 (s, 1H), 7.75 (s, d, 3H), 7.16 (d, 1H), 3.86 (d, 9H), 2.28 (t, 2H), 1.4 (t, 2H), 1.23 (s, 31H), 0.85 (t, 3H).

### (ii) Preparation of Compound No. 61

5-(4-Methoxycarbonyl-2-octadecanoylamino-phenoxy)-isophthalic acid dimethyl ester obtained in (i) (750 mg, 1.2 mmol), was dissolved in 1,4-dioxane:MeOH (75:25), and 1M NaOH (5 ml, 5 mmol) was added to the solution. The mixture was stirred at 25° C. for 48 hrs. 250 ml of cold water was added to the mixture and 1M HCl was added to the mixture until pH=1 was achieved. The substance was extracted with EtOAc several times. The organic layer was washed with water and brine, dried over MgSO₄ and was concentrated. The title compound was collected (480 mg, 0.82 mmol) in 69% yield. ¹H NMR (DMSO): δ 8.6 (s, 1H), 8.24 (s, 1H), 7.68 (m, 3H), 7.1 (d, 1H), 2.3 (t, 2H), 1.45 (s, 2H), 1.23 (s, 27H), 0.85 (t, 3H).

### Example 2

### Preparation of Compound No. 70

For the preparation of Compound No. 70, 4-sulfophenyl isothiocyanate (38 mg, 0.15 mmol) was added to a solution of octadecylamine (20 mg, 0.074 mmol) in 2 ml DMF. The reaction mixture was stirred at 50° C. for 20 hrs. The reaction mixture was cooled to 20° C. and the precipitation that was formed was filtered and recrystallized in hot ethanol, thus obtaining the title compound (18 mg, 52% yield). ¹H NMR (DMSO): δ 9.45 (br s, 1H), 7.75 (br s, 1H), 7.52 (d, 2H), 7.32 (d, 2H), 3.42 (m, 2H), 1.53 (m, 2H), 1.24 (m, 30H), 0.85 (t, 3H)

### Example 3

### Preparation of Compound No. 71

For the preparation of Compound No. 71, 5-(3-amino-5-oxo-2-pyrazolin-1-yl)-2-phenoxy-benzenesulfonic acid (600 mg, 1.7 mmol) was dissolved in 150 ml of acetonitrile and lauroyl chloride (1.2 ml, 5.1 mmol) and triethylamine (0.36 ml, 2.6 mmol) were added. The mixture was refluxed for 3 hrs. The mixture was poured into water and the solvent was evaporated. The product was purified by liquid chromatography (RP-18) with MeOH:Water (1:1) as eluents. The title compound (128 mg, 0.2 mmol) was collected in 14% yield. ¹H NMR (DMSO): δ 8.2 (d, 1H), 7.63 (dd, 1H), 7.32 (t, 2H), 7.05 (t, 1H), 6.94 (d, 2H), 6.85 (d, 1H), 2.26 (m, 2H), 1.54 (m, 2H), 1.24 (s, 16H), 0.85 (t, 3H)

### Example 4

### Preparation of Compound No. 83

For the preparation of Compound No. 83, 5-(3-amino-5-oxo-2-pyrazolin-1-yl) 2-phenoxy-benzene sulfonic acid (100 mg, 0.3 mmol) was dissolved in 20 ml of dry acetonitrile with triethylamine (0.19 ml, 1.7 mmol) and myristoyl chloride (0.19 ml, 0.7 mmol) was added. The mixture was refluxed for 1 hr. The mixture was poured into 20 ml of water and the acetonitrile was evaporated. To the solution, 2M HCl was added until a pH of 2-3 was achieved. The product was filtered with MN GF-1 filter paper with suction. The substance was purified by chromatography and was eluted with CH₂Cl₂: MeOH (9.5:0.5). The title compound (77 mg, 0.14 mmol) was obtained in 48% yield. ¹H NMR (CD₃OD) δ 8.45 and 8.3 (d, 1H), 7.94 and 7.68 (dd, 1H), 7.37 (q, 1H), 7.12 (m, 3H), 6.88 (t, 1H), 2.35 (t, 2H), 1.65 (m, 2H), 1.28 (s, 25H), 0.89 (t, 3H). MS m/z (ES) 391 (MH⁺).

### Example 5

### Preparation of Compound No. 84

For the preparation of Compound No. 84, 5-(3-amino-5-oxo-2-pyrazolin-1-yl)-2-phenoxy-benzenesulfonic acid (50 mg, 0.1 mmol) was dissolved in 20 ml of dry acetonitrile. Pentadecanoyl chloride (0.08 ml, 0.3 mmol) and triethylamine (0.02 ml, 0.1 mmol) were added. The mixture was refluxed for 1 hr. The mixture was poured into water and the product was filtered. The substance was purified by chromatography with CH₂Cl₂: 15% MeOH as eluents. The title compound (30 mg, 0.05 mmol) was collected in 38% yield. ¹H NMR (DMSO): δ 8.2 (d, 1H), 7.6 (m, 1H), 7.32 (t, 2H), 7.04 (t, 1H), 6.9 (d, 2H), 6.8 (d, 1H), 2.25 (m, 2H), 1.53 (t, 2H), 1.24 (s, 20H), 0.85 (t, 3H). MS m/z (FAB) 610 (MK⁺).

### Example 6

### Preparation of Compound No. 85

For the preparation of Compound No. 85, petroselinic acid (56 mg, 0.2 mmol) was dissolved in 3 ml dichloromethane and 1-hydroxybenzotriazole (HOBt; 27 mg, 0.2 mmol), EDC (76 mg, 0.4 mmol) and Et₃N (40 mg, 0.4 mmol) were added consecutively. After 10 min, the amine 5-(3-amino-5-oxo-2-pyrazolin-1-yl)-2-phenoxy-benzenesulfonic acid (57 mg, 0.2 mmol) was added and the reaction mixture was allowed to react at 25° C. for 20 hr. Then, dichloromethane (5 ml) was added and the mixture was washed with 2M HCl (5 ml) and water, dried over MgSO₄ and evaporated to give a brown solid. Chromatography using 15% MeOH in CH₂Cl₂ as eluent gave the title compound (15.2 mg, 0.025 mmol) in 12.5% yield. ¹H NMR (CD₃OD): δ 7.83 (s, 1H), 7.55 (d, 1H). 7.44 (m, 2H), 7.31 (d, 2H), 7.09, (t, 1H), 6.78 (d, 1H), 5.34 (m, 2H), 2.55 (t, 2H), 2.05 (q, 2H), 2.00 (q, 2H), 1.67 (pent, 2H), 1.60 (pent, 2H), 1.26 (m, 18H), 0.87 (t, 3H); MS m/z (FAB) 650 (MK⁺).

### Example 7

### Preparation of Compound No. 86

For the preparation of Compound No. 86, petroselinic acid (141 mg, 0.5 mmol), was dissolved in 3 ml 1,4-dioxane and dimethyl 5-(2-amino-4-(methoxycarbonyl)phenoxy)isophthalate (178 mg, 0.5 mmol) and pyridine (40 mg, 0.5 mmol) were added. Di-t-butyl dicarbonate (BOC₂O; 142 mg, 0.65 mmol) dissolved in 1 ml dioxane was added. After stirring at 25° C. for 10 min, the mixture was heated in oil-bath at 80° C. overnight. The solvent was evaporated and chromatography using hexane:ethylacetate (EtOAc) (8:2) as eluent gave the triester-amide derivative (233 mg, 0.37 mmol) in 75% yield. The latter compound (50 mg, 0.093 mmol) was hydolysed by 1M NaOH (0.5 ml) in 1,4-dioxand (4 ml) and MeOH (1 ml) for 2 hr at 25° C. The mixture was acidified to pH 1 with 1M HCl and extracted by EtOAc to give the amide-tricarboxylic acid derivative title compound (53 mg, 0.091 mmol) in 98% yield (73.5% for 2 steps). ¹H NMR (CD₃OD): δ 9.05 (d, 1H), 8.57 (t, 1H). 7.92 (d, 2H), 7.75 (dd, 1H), 6.82, (d, 1H), 5.34 (m, 2H), 2.42 (t, 2H), 2.05 (q, 2H), 2.00 (q, 2H), 1.75 (pent, 2H), 1.43 (pent, 2H), 1.26 (m, 18H), 0.87 (t, 3H); MS m/z (FAB) 582 (MH⁺).

### Example 8

### Preparation of Compound No. 87

For the preparation of Compound No. 87, petroselinic acid was reacted with dimethyl 5-(2-amino-4-(methoxycarbonyl)phenoxy)isophthalate to give triester-amide derivative (75% yield) as described above for the preparation of Compound No. 86. The resulting compound (62 mg, 0.1 mmol) was dissolved in 2 ml dichloromethane and m-chloroperbenzoic acid (mCPBA; 70%) was added as a solid (25 mg, 0.14 mmol) and the mixture was stirred at 25° C. After 2 hr, dichloromethane (8 ml) was added and the mixture was washed by 5% NaHCO₃ and water, dried over sodium sulfate and evaporated. Purification of the epoxide product was carried out using hexane:dichloromethane (DCM):EtOAc (8:1:1) to give the triester-amide epoxide derivative as colorless oil-solid (52 mg, 0.081 mmol) in 81% yield. The latter compound (15 mg, 0.02347 mmol) was hydrolyzed by 1M NaOH (0.25 ml) in 1,4-dioxane (2 ml) and MeOH (0.5 ml) for 2.5 hr at 25° C. The mixture was acidified to pH 1 with 5% NaHSO₄ and extracted by EtOAc to give the epoxide-amide-tricarboxylic acid derivative title compound (14 mg, 0.02345 mmol) in 99% yield (60% for 3 steps). ¹H NMR (CD₃OD): δ 8.62 (d, 1H), 8.43 (t, 1H). 7.86 (dd, 1H), 7.84 (d, 2H), 7.04, (d, 1H), 2.88 (m, 2H), 2.40 (t, 2H), 1.66 (q, 4H), 1.50 (pent, 2H), 1.46 (pent, 2H), 1.26 (m, 18H), 0.87 (t, 3H); MS m/z (ES) 598 (MH⁺).

### Example 9

### Preparation of Compound No. 90

Compound No. 90 was prepared starting from 2-(1-eicosenyl)-4,6 dimethoxycarbonyldibenzofuran as follows:

### (i) Preparation of 2-(1-eicosenyl)-4,6 dimethoxycarbonyldibenzofuran

To a mixture of dry potassium carbonate (70 mg, 0.51 mmol), tetrabutylammonium chloride (55.7 mg, 0.20 mmol), 2-iodo-4,6-dimethoxycarbonyldibenzofuran (70 mg, 0.17 mmol) and palladium acetate (1.4 mg, 0.006 mmol) at 20° C. under argon, a solution of 1-eicosene (239 mg, 0.85 mmol) in 2.8 ml dry DMF was added. The reaction mixture was heated to 100° C. and stirred for 5 hrs. After cooling, the reaction mixture was extracted with ethyl acetate and water. After evaporation of the solvent the residue was purified by chromatography using hexane-EtOAc (95:5) as eluents. 63 mg (65.8% yield) of 2-(1-eicosenyl)-4,6 dimethoxycarbonyldibenzofuran was obtained. ¹H NMR (CDCl₃) δ 8.15 (m, 4H), 7.45 (t, 1H), 6.45 (m, 2H), 4.11 (s, 6H), 2.27 (m, 2H), 1.25 (m, 32H), 0.87 (t, 3H); MS (dci/i-bu) m/z 562

### (ii) Preparation of Compound No. 90

For the preparation of Compound No. 90, to a stirred solution of 2-(1-eicosenyl)-4,6-dimethoxycarbonyl-dibenzofuran obtained in (i) (23 mg, 0.041 mmol) in 4 ml ethanol, 1.5 ml of 2N NaOH was added. The reaction mixture was stirred for 3 hrs at 20° C. and for 2 hrs at 40° C. To the reaction mixture, 0.5 ml of 10N HCl was added. This mixture was evaporated under vacuum and the residue was diluted with 10 ml CH₂Cl₂. Inorganic salts were filtered and the filtrate was evaporated under vacuum. This work-up was repeated twice and 11 mg (50.4% yield) of the title compound was isolated. MS (DCI/CH₄) m/z 535 (MH⁺).

### Example 10

### Preparation of Compound No. 91

Compound No. 91 was prepared starting from 3,6-dibromo-9-oxiranylmethyl-9H-carbazole as follows:

### (i) Preparation of 3,6-dibromo-9-oxiranylmethyl-9H-carbazole

3,6-dibromocarbazole (500 mg, 1.5 mmol) was dissolved in 25 ml of dry acetonitrile. Potassium carbonate (415 mg, 3 mmol) and 6.2 ml of epichlorohydrin were added. The mixture was refluxed for 4 hrs. 75 ml of water were added and 100 ml of CH₂Cl₂, and the organic phase was extracted. 50 ml of 0.2 M HCl were added to the organic phase and the organic phase was extracted. The organic solution was kept in the refrigerator for 12 hours and the formed precipitation was filtered. The 3,6-dibromo-9-oxiranylmethyl-9H-carbazole product (282 mg, 0.74 mmol) was collected in 49% yield. ¹H NMR (CDCl₃): δ 8.13 (s, 2H), 7.57 (d, 2H), 7.34 (dd, 2H), 4.67 and 4.25 (dd, 2H), 3.32 (m, 1H), 2.82 and 2.48 (t, q, 2H).

### (ii) Preparation of Compound No. 91

For the preparation of Compound No. 91, the compound 3,6-dibromo-9-oxiranylmethyl-9H-carbazole obtained in (i) (0.05 mg, 0.13 mmol) was dissolved in 4 ml of EtOH. Octadecylamine (42 mg, 0.16 mmol) was added and the mixture was refluxed for 12 hrs. The crude product was purified by chromatography with 5% MeOH in CH₂Cl₂ as eluents. The title compound (42 mg, 0.06 mmol) was collected in 50% yield. ¹H NMR (DMSO): δ 8.4 (d, 2H), 7.64 (d, 2H), 7.58 (dd, 2H), 4.47 and 4.3 (dd, 2H), 3.9 (br s, 2H), 2.64-2.48 (m, 4H), 1.43 (t, 2H), 1.22 (s, 31H), 0.85 (t, 3H). MS m/z (ES) 649, 651 and 653 (MH⁺).

### Example 11

### Preparation of Compound No. 92

For the preparation of Compound No. 92, petroselinic acid was reacted with dimethyl 5-(2-amino-4-(methoxycarbonyl)phenoxy)isophthalate to give triester-amide derivative (75% yield), as described in Example 7 (preparation of Compound 86). The resulting amide was epoxidized by m-chloroperbenzoic acid (mCPBA) (81% yield) as described in Example 8 (preparation of Compound No. 87). The amide-epoxide derivative (27 mg, 0.043 mmol) was dissolved in 2 ml dichloromethane and dimethylthioformamide (DMTF; 8.4 mg, 8 μl, 0.091 mmol) was added, followed by addition of one drop of TFA (catalytic amount) and the mixture was stirred at 25° C. After 48 hr, dichloromethane was evaporated and the residue was dissolved in hexane with few drops of dichloromethane (for homogeneousness). The mixture was washed 3 times with water, dried over sodium sulfate and evaporated. Purification of the thiirane product was carried out using hexane:dichloromethane:EtOAc:Et₃N (7:2:1:0.05) to give the triester-amide thiirane derivative as a white solid (21.5 mg, 0.033 mmol) in 76% yield. The later (6.6 mg, 0.01 mmol) was hydrolysed by 1 M NaOH as described in the preparation of Compound No. 87 and acidified by 5% NaHSO₄ to pH 3 leading to the thiirane-amide-tricarboxylic acid title compound as a white solid (4.7 mg, 0.0076 mmol) in 76.6% yield (35% for 4 steps). ¹H NMR (CD₃OD): δ 8.60 (d, 1H), 8.43 (t, 1H), 7.86 (dd, 1H), 7.84 (d, 2H), 7.06 (d, 1H), 3.08 (m, 2H), 2.41 (t, 2H), 1.62 (m, 6H), 1.55 (pent, 2H), 1.27 (m, 18H), 0.89 (t, 3H).

### Example 12

### Preparation of Compound No. 93

For the preparation of Compound No. 93, the three steps used in the preparation Compound No. 87 (Example 8) were repeated starting by reacting petroselinic acid with dimethyl 5-(2-amino-4-(methoxycarbonyl)phenoxy) isophthalate. The only difference was in the hydrolysis step (last step) wherein 2 M HCl was added to obtain pH 0 (instead of 5% NaHSO₄). This modification led to opening of the epoxide group to form the hydrochlorine derivative title compound (80% yield for the last step; 48.6% for 3 steps). The structure of Compound No. 93 was confirmed (existence of Cl atom) by MS analysis. ¹H NMR (CD₃OD): δ 8.60 (d, 1H), 8.44 (t, 1H), 7.86 (d, 2H), 7.84 (dd, 1H), 7.02, (d, 1H), 3.82 (m, 1H), 3.58 (m, 1H), 2.40 (t, 2H), 1.76 (q, 4H), 1.54 (pent, 2H), 1.26 (m, 20H), 0.87 (t, 3H); MS m/z (ES) 634, 636 (MH⁺).

### Example 13

### Preparation of Compound No. 94

For the preparation of Compound No. 94, 7-oxo-heptadecenoic acid (26 mg, 0.092 mmol) was dissolved in 3 ml 1,4-dioxane, and dimethyl 5-(2-amino-4-(methoxycarbonyl)phenoxyisophthalate (43 mg, 0.12 mmol) and pyridine (14 mg, 0.17 mmol) were added. Di-t-butyl dicarbonate (BOC₂O; 44 mg, 0.22 mmol) dissolved in 1 ml dioxane was added. After stirring at 25° C. for 10 min the mixture was heated in oil-bath at 80° C. overnight. The solvent was then evaporated and chromatography using hexane:dichloromethane:EtOAc (8:1:1) as eluent gave the triester-amide derivative (21 mg, 0.033 mmol) in 36% yield. The latter compound (11 mg, 0.017 mmol) was hydrolyzed by 1 M NaOH (0.5 ml) in 1,4-dioxand (4 ml) and MeOH (1 ml) for 7 hr at 25° C. The mixture was acidified to pH 1 with 5% NaHSO₄ and extracted by EtOAc to give the amide-tricarboxylic acid derivative title compound (8 mg, 0.013 mmol) in 81% yield (29% for 2 steps). ¹H NMR (CD₃OD): δ 8.59 (d, 1H), 8.44 (t, 1H), 7.85 (dd, 1H), 7.84 (d, 2H), 7.05 (d, 1H), 5.79 (ddq, 1H), 4.96 (dq, 1H), 4.90 (dq, 1H), 2.40 (t, 2H), 2.37 (t, 2H), 2.36 (t, 2H), 2.03 (m, 2H), 1.55 (pent, 2H), 1.50 (m, 6H), 1.28 (m, 8H); MS m/z (FAB) 582 (MH⁺).

### Example 14

### Preparation of Compound No. 95 and Compound No. 96

For the preparation of Compounds Nos. 95 and 96, the triester-amide derivative (75% yield) obtained in Example 7, was epoxidized by mCPBA (81% yield) as described in Example 8. The amide-epoxide derivative (45 mg, 0.07 mmol) was dissolved in 1 ml NH₃-MeOH (ca. 7N) and the mixture was transferred to a special tube (bomba), sealed and heated to 80° C. for 48 hr. After cooling, the solvent was evaporated and two products were purified by chromatography. The use of hexane: EtOAc (8:2) as eluent gave diester product (17 mg, 0.027 mmol) as colorless oil in 39% yield and monoester (9 mg, 0.014 mmol) in 20% yield as colorless oil. The diester compound (11 mg, 0.017 mmol) was hydrolyzed by 1 M NaOH as described in Example 8, leading to Compound No. 95 as a white solid (9.2 mg, 0.015 mmol) in 87% yield (20% for 4 steps). In the same manner, the mono-ester derivative (6.6 mg, 0.011 mmol) was hydrolyzed to give Compound No. 96 as a white solid (5.4 mg, 0.009 mmol) in 82% yield (10% for 4 steps). Compound No. 95: ¹H NMR (CD₃OD): δ 9.14 (d, 1H), 9.08 (br s, 2H), 8.39 (t, 1H), 7.86 (t, 1H), 7.80 (t, 1H), 7.78 (dd, 1H), 7.06, (d, 1H), 2.81 (m, 2H), 2.49 (t, 2H), 1.72 (q, 4H), 1.50 (pent, 2H), 1.42 (pent, 2H), 1.28 (m, 18H), 0.87 (t, 3H); MS m/z (FAB) 597 (MH⁺).

Compound No. 96: ¹H NMR (CD₃OD): δ 8.53 (d, 1H), 8.19 (t, 1H), 7.88 (dd, 1H), 7.70 (d, 2H), 7.04, (d, 1H), 2.90 (m, 2H), 2.40 (t, 2H), 1.67 (q, 2H), 1.53 (pent, 2H), 1.49 (m, 4H), 1.29 (m, 18H), 0.90 (t, 3H); MS m/z (FAB) 596 (MH⁺).

### Example 15

### Preparation of Compound No. 98

For the preparation of Compound No. 98, 2-trifluoromethyl-2-hydroxy-trans-octadecenoic acid (85 mg, 0.23 mmol) was dissolved in 3 ml 1,4-dioxane, and dimethyl 5-(2-amino-4-(methoxycarbonyl)phenoxy)isophthalate (215 mg, 0.6 mmol) and pyridine (79 mg, 1 mmol) were added. To this solution, di-t-butyl dicarbonate (BOC₂O; 218 mg, 1 mmol) was added dissolved in 1 ml dioxane and the mixture was stirred at 80° C. overnight. The solvent was evaporated and chromatography using hexane:EtOAc (1:1) as eluent gave the triester-amide as colorless oil (15 mg, 0.02 mmol) in 9% yield. The latter compound (8.3 mg, 0.012 mmol) was hydrolyzed by 1 M NaOH, as described in Example 8, leading to the title compound as a white solid (5.8 mg, 0.0087 mmol) in 72.7% yield (6.5% for 2 steps). ¹H NMR (CD₃OD): δ 8.46 (d, 1H), 8.38 (t, 1H), 7.89 (d, 2H), 7.82 (dd, 1H), 7.00 (d, 1H), 5.55 (m, 1H), 5.38 (m, 1H), 3.21 (m, 1H), 2.84 (m, 1H), 2.01 (m, 2H), 1.29 (m, 22H), 0.90 (t, 3H).

### Example 16

### Preparation of Compound No. 99

For the preparation of Compound No. 99, petroselinic acid (546 mg, 2 mmol) was dissolved in 10 ml dichloromethane, mCPBA (70%) was added as a solid (738 mg, 3 mmol) and the mixture was stirred at 25° C. After 2 hrs, half of the solvent was evaporated and the formed precipitate was filtered and washed with cold dichloromethane. The solvent was evaporated and chromatography using dichloromethane:EtOAc (8:2) gave epoxide derivative (150 mg, 0.5 mmol) in 25% yield. The latter compound (85 mg, 28 mmol) was dissolved in 3 ml MeCN and BOC₂O (109 mg, 0.5 mmol), 3-hydroxy-3,4-dihydrobenzotriazin-4-one (HODhbt; 66 mg, 0.4 mmol), Et₃N (33 mg, 0.33 mmol) and dimethylaminopyridine (DMAP; 20 mg, 0.165 mmol) were added consecutively. The reaction was stirred at 25° C. for 5 hr. Dichloromethane was added and the mixture was washed 2 times with 5% NaHCO₃, with 0.25 M HCl, dried (Na₂SO₄) and evaporated. Chromatography using hexane:EtOAc (9:1) as eluent gave the active ester title compound as a pale solid (45 mg, 0.1 mmol) in 36% yield (9% for 2 steps). ¹H NMR (CDCl₃): δ 8.38 (d, 1H), 8.24 (d, 1H), 8.02 (t, 1H), 7.85 (t, 1H), 2.94 (m, 2H), 2.81 (t, 2H), 1.95 (pent, 2H), 1.68 (m, 2H), 1.63 (m, 2H), 1.53 (m, 2H), 1.26 (m, 18H), 0.88 (t, 3H).

### Example 17

### Preparation of Compound No. 100

For the preparation of Compound No. 100, petroselinic acid was reacted with mCPBA as described in Example No. 16 (preparation of Compound No. 99). The epoxy-petroselinic acid (75 mg, 0.25 mmol) was dissolved in 2 ml dry dichloromethane and 1-hydroxybenzotriazole (HOBt: 34 mg, 0.25 mmol), N-ethyl-N′-(3-dimethylaminopropyl)carbodiimide hydrochloride (EDC-HCl; 72 mg, 0.375 mmol) and Et₃N (25 mg, 0.25 mmol) were added. In another flask, amine 5-(3-amino-5-oxo-2-pyrazolin-1-yl)-2-phenoxybenzenesulfonic acid (57 mg, 0.2 mmol) was dissolved in 2 ml dry dichloromethane and Et₃N (50 mg, 0.5 mmol) was added. The solution of the amine was added dropwise to the first solution (red color). The mixture was stirred at 25° C. for 72 hr. Dichloromethane (20 ml) was added and the mixture was washed with 5% NaHSO₄ (5 ml of isopropanol were added), dried over Na₂SO₄ and evaporated to give reddish oil. Chromatography using EtOAc:MeOH (85:15) gave the epoxy-amide title compound as a pale yellow solid (30.5 mg, 0.048 mmol) in 19.5% yield (4.5% for 2 steps). ¹H NMR (CD₃OD): δ 8.69 (d, 1H), 7.67 (dd, 1H), 7.32 (t, 2H), 7.08, (m, 3H), 6.83 (d, 1H), 3.40 (m, 2H), 2.75 (t, 2H), 1.73 (q, 2H), 1.54 (pent, 2H), 1.49 (m, 4H), 1.29 (m, 18H), 0.89 (t, 3H); MS m/z (dci/ch₄) 649 ((M−H)Na).

### Example 18

### Preparation of Compound No. 101

Compound 101 was prepared starting from 3-nitro-4-nonadecylamino-benzenesulfonic acid, as follows:

### (i) Preparation of 3-nitro-4-nonadecylamino-benzenesulfonic acid

4-Chloro-3-nitrobenzenesulfonic acid sodium salt (2.5 gr, 7.7 mmol), octadecylamine (2.1 gr, 7.7 mmol) and NaHCO₃ (0.65 gr, 7.7 mmol) were dissolved in 11.5 ml of water, 9.6 ml of butanol and 2.4 ml of methanol. The mixture was refluxed for 20 hrs. After evaporation of the solvent the residue was stirred with 125 ml of hot methanol. The mixture was cooled and filtered and 2.74 g (5.6 mmol) of 3-nitro-4-nonadecylamino-benzenesulfonic acid was obtained in 72% yield. ¹H NMR (DMSO) δ 8.24 (d, 1H), 7.69 (d, 1H), 7.03 (d, 1H), 1.61 (br t, 2H), 1.23 (s, 32H), 0.85 (t, 3H). MS m/z (CI) 391 (MH⁺).

### (ii) Preparation of Compound No. 101

For the preparation of Compound No. 101, 3-nitro-4-nonadecylamino-benzenesulfonic acid (1.8 gr, 3.6 mmol) obtained in (i), was dissolved in 15 ml of hot glacial acetic acid and 6 ml of MeOH. This solution was slowly added while stirring to 7.4 ml of concentrated HCl and SnCl₂ H₂O (4.4 gr, 19 mmol). The mixture was heated to 65° C. for 16 hours. The mixture was then cooled to 25° C. and filtered with suction. The title compound (1.19 gr, 2.7 mmol) was obtained in 75% yield. ¹H NMR (DMSO): δ 6.8 (d, 1H), 6.7 (dd, 1H), 6.2 (d, 1H), 4.5 (s, 2H), 4.4 (t, 1H), 2.18 (q, 2H), 1.57 (q, 2H), 1.23 (s, 30), 0.85 (t, 3H). MS m/z (CI) 360.

### Example 19

### Preparation of Compound No. 102

For the preparation of Compound No. 102, Compound No. 101 (100 mg, 0.2 mmol) obtained in Example 18, was suspended in 5 ml of dry benzene and in 0.06 ml (0.76 mmol) of dry pyridine. In order to remove water, 1 ml of benzene was distilled off. Benzoyl chloride (0.09 ml, 0.76 mmol) was added and benzene was removed by distillation. The reaction mixture was heated at 110° C. for 1 hr and another 2 hrs at 130° C. Two and a half (2.5) ml of glacial acetic acid was added and the reaction mixture was heated at 120° C. for another 30 minutes. After cooling the mixture to less than 80° C., 1.5 ml of EtOH was added and the mixture was stirred at 25° C. for 10 hrs. The solvent was evaporated and the product was recrystallized from EtOH. The title compound (70 mg, 0.13 mmol) was collected in 60% yield. ¹H NMR (CD₃OD): δ 8.24 (s, 1H), 8.13 (dd, 1H), 8.05 (dd, 1H), 7.9 (dd, 2H), 7.84-7.72 (m, 3H), 4.54 (t, 2H), 3.59 (q, 2H), 1.89 (t, 2H), 1.28 (s, 28H), 0.89 (t, 3H). MS m/z (FAB) 527 (MH⁺).

### Example 20

### Preparation of Compounds Nos. 103 and 104

For the preparation of Compounds Nos. 103 and 104, 4-phenoxyaniline (1 g, 5.4 mmol) and itaconic acid (0.74 g, 5.7 mmol) were mixed together and placed in 250 ml rounded-bottom flask. The flask was heated in oil-bath at 250° C. (stirring) for 10 min, leading to hard solid. The product was crystallized from EtOAc to give N-aryl 4-carboxypyrrolidinone as a white solid (1.178 g, 3.96 mmol) in 73% yield. The latter product (297 mg, 1 mmol) was dissolved in dry THF (20 ml) and EDC-HCl (288 mg, 1.5 mmol), HOBt (135 mg, 1 mmol), and Et₃N (303 mg, 3 mmol) were added consecutively. After 10 min, octadecylamine (404 mg, 1.5 mmol) dissolved in 10 ml of dry dichloromethane was added and the mixture was allowed to react at room temperature overnight. Then, dichloromethane (30 ml) was added and the mixture wad washed with 5% NaHSO₄, 10% NaHCO₃ and with water, dried over Na₂SO₄ and evaporated to give yellow solid. The product N-aryl 4-carboxamide-pyrrolidinone was crystallized from EtOAc to give off-white solid (306 mg, 0.56 mmol) in 56% yield. The amide-pyrrolidinone (110 mg, 0.2 mmol) was placed in rounded-bottom, flask and concentrated H2SO₄ (2 ml) was added. While stirring, the mixture was heated in an oil-bath at 100° C., in which the starting amide was totally dissolved. The heating was continued for 5 hr. After cooling, cold water (10 ml) was added leading to precipitation. The solid was filtered and washed with water. Purification was carried out by reverse phase chromatography using H₂O:MeOH (4:6) as eluent leading to two products: monosulfonated Compound No. 103 as a white solid (17.9 mg, 0.028 mmol) in 14.2% yield, and the more polar disulfonated Compound No. 104 as a white solid (14.7 mg, 0.02 mmol) in 10.4% yield. ¹H NMR Compound No. 103 (CD₃OD): δ 8.08 (t, 1H), 7.66 (d, 2H), 7.58 (d, 2H), 7.05 (d, 2H), 6.89 (d, 2H), 3.98 (t, 1H), 3.83 (dd, 1H), 3.20 (pent, 1H), 3.07 (q, 2H), 2.65 (m, 2H), 1.40 (pent, 2H), 1.23 (br s, 30H), 0.85 (t, 3H); MS m/z (ES) 629 (MH⁺).

¹H NMR Compound 104 (CD₃OD): δ 8.47 (br s, 1H), 7.81 (d, 1H), 7.62 (d, 2H), 7.16 (d, 2H), 6.87 (d, 1H), 4.07 (t, 1H), 4.00 (dd, 1H), 3.32 (pent, 1H), 3.21 (t, 2H), 2.81 (m, 2H), 1.53 (pent, 2H), 1.29 (br s, 30H), 0.89 (t, 3H); MS m/z (ES) 707 (MH⁻).

### Example 21

### Preparation of Compound No. 105

For the preparation of Compounds No. 105, itaconic acid (185 mg, 1 mmol) was added to a 100 ml flask containing 4-phenoxyaniline (185 mg, 1 mmol). The flask containing the solid mixture was heated in an oil-bath at 150° C. for 5 min, leading to a colorless solid. The crude solid was refluxed in EtOAc (20 ml) until fully dissolved (2 h). A short time after cooling, a white precipitate appeared. The mixture was allowed to stand at 25° C. for few hours and then filtered to give 192 mg (65% yield) of 5-oxo-1-(4-phenoxy-phenyl)-pyrrolidine-3-carboxylic acid as a colorless solid. The later product (297 mg, 1 mmol), was dissolved in THF (dry, 20 ml) and dry dichloromethane (DCM) (10 m39l), and EDC-HCl (288 mg, 1.5 mmol), HOBt (135 mg, 1 mmol) and Et₃N (303 mg, 3 mmol) were added consecutively and the mixture was stirred for 10 min. The amine (404 mg, 1.5 mmol) was then added and the mixture was allowed to react at 25° C. overnight. Dichloromethane (DCM) was added and the mixture was washed with NaHSO₄, NaHCO₃ and water, dried, and evaporated to give a yellow solid (606 mg crude). The solid was crystallized from 30 ml EtOAc. Filtration gave 306 mg (56% yield) of 5-oxo-1-(4-phenoxy-phenyl)-pyrrolidine-3-carboxylic acid octadecylamide as a pale solid. The later product (110 mg, 0.2 mmol), was then dissolved in 2 ml DCM and 1 ml trifluoroacetic acid (TFA). Br₂ in 1 ml DCM and 0.5 ml TFA was added to the solution. The red solution thus obtained, was stirred for 3 days at 25° C. Then water was added and the organic layer was washed 3 times with NaHCO₃. The organic layer was evaporated to give a white solid. Recrystallization from EtOH (10 ml) gave 1-[4-(4-bromo-phenoxy)-phenyl]-5-oxo-pyrrolidine-3-carboxylic acid octadecylamide as a white solid (59 mg; 47% yield).

H₂SO₄ (1.6 ml) and TFA (1.6 ml) were added to the later product (54 mg, 0.086 mmol) thus obtained, and the mixture was stirred at 25° C. for 24 hr. Then cold water was added and a white solid precipitated. Centrifugation gave a white solid, which dissolved well in EtOH. RP-chromatography (H₂O:MeOH, 6:4, 1:1, 4:6, 3:7, 2:8 and washing by 1:9) gave 19.1 mg of Compound No. 105 in 31.4% yield. ¹H NMR (cd3od): [symbol] 8.05 (D, 1H), 7.59 (D, 1H), 7.49 (D, 2H), 7.13 (D, 2H), 6.77 (D, 1H), 4.06 (T, 1H), 3.98 (DD, 1H), 3.31 (PENT, 1H), 3.21 (T, 2H), 2.80 (M, 2H), 1.52 (PENT, 2H), 1.28 (BR S, 30H), 0.90 (T, 3H); ms M/Z (es) 705 and 707 (mh⁻).

### Example 22

### Preparation of Compound Nos. 106 and 107

For the preparation of Compounds Nos. 106 and 107, NaH (60%; 1.2 g, 30 mmol) was suspended in 4 ml dry toluene, and 10 ml dry DMSO was added. 4-cyanophenol (2.43 g, 20.4 mmol) in 10 ml DMSO was added carefully and hydrogen evolution appeared and ceased. The mixture was then heated to 80° C. and 1-fluoro-4-nitrobenzene (2.82 g, 20 mmol) in 5 ml DMSO was added. The mixture was stirred overnight (dark color). After cooling, the mixture was transferred into two centrifuge bombes and the solid was separated, washed with water and separated again. The solid was then dissolved in DCM and the organic layer was washed with water (to remove DMSO). Crystallization from EtOH (30 mL)/CHCl₃ (30 ml) and filtration, gave 4-(4-nitro-phenoxy)-benzonitrile as a golden solid (2.282 g, 47.5%). The filtrate was evaporated (HPLC, 77%) to give 1.590 mg, 25.4% (total yield 73.0%).

To hot a solution of the later product (1.22 g, 5.1 mmol) in AcOH/MeOH (25/40 ml), SnCl-H₂O (6.0 g, 26.5 mmol) solution in concentrated HCl (5 ml) was added. The mixture was stirred at 70° C. for 3h. After cooling, 2N NaOH (100 ml) was added until pH 11 was achieved. EtOAc (100 ml) was then added while stirring, and the solution became red. The organic layer was dried and evaporated to give a dark green solid. Chromatography (silica, DCM and 5% EtOAc in DCM) gave the 4-(4-cyano-phenoxy)aniline (591 mg, 55%) as off-white solid. To this product (498 mg, 2.37 mmol) contained in a 100 ml flask, itaconic acid (370 mg, 2.85 mmol) was added, and the solid mixture was heated in an oil-bath at 150° C. After 30 min, when no evolution of water was observed anymore, the bath was heated to 180° C. for 30 min, leading to brown hard oil. The crude product was well dissolved in EtOAc and extracted with 2N NaOH three times. The aqueous layer was acidified with concentrated HCl to pH 2 and washed twice with EtOAc. The organic layer was washed by water, dried over sodium sulfate and evaporated to give 1-[4-(4-cyano-phenoxy)-phenyl]-5-oxo-pyrrolidine-3-carboxylic acid as a bright yellow solid; 535 mg (70%). THF (dry, 20 ml) and dry DCM (10 ml) were to the later crude product (400 mg, 1.24 mmol), then EDC-HCl (480 mg, 2.5 mmol), HOBt (168 mg, 1.24 mmol) and Et₃N (278 mg, 3.72 mmol) were added consecutively, and the mixture was stirred for 10 min. The amine (673 mg, 2.5 mmol) was added and the mixture was allowed to react at room temperature overnight. Then DCM was added and the mixture was washed with NaHSO₄, NaHCO₃, and water, dried and evaporated to give yellow solid (870 mg crude). The solid was crystallized from 40 ml EtOAc. Filtration gave 351 (49.4%) of (1-[4-(4-cyano-phenoxy)-phenyl]-5-oxo-pyrrolidine-3-carboctadecylamide) as a pale solid.

H₂SO₄ (2 ml) was added to the later product and the mixture was heated at 90° C. for 4 days. Cold water was added leading to a white precipitate. Centrifugation gave a white solid. RP-Chromatography (H₂:MeOH) using 6:4, 1:1, 4:6, 3:7, 2:8 and washing by 1:9, gave 2 products; the first, Compound 106 (2-(4-amido-phenoxy)-5-(4-octadecyl-carbamoyl-2-oxo-pyrrolidin-1-yl)-benzene sulfonic acid) (10.3 mg, 6% yield) and the second, Compound 107 (2-(4-carboxy-phenoxy)-5-(4-octadecyl-carbamoyl-2-oxo-pyrrolidin-1-yl)-benzene sulfonic acid) (4.1 mg, 2.5% yield).

Compound 106: ¹H NMR (CD₃OD): δ 8.09 (d, 1H), 7.85 (d, 2H), 7.84 (dd, 1H), 7.07 (d, 2H), 7.00 (d, 1H), 4.12 (t, 1H), 4.01 (dd, 1H), 3.31 (pent, 1H), 3.21 (t, 2H), 2.81 (m, 2H), 1.53 (pent, 2H), 1.28 (br s, 30H), 0.90 (t, 3H); MS m/z (ES⁻) 670 (MH⁻, 48), 212 (100).

Compound 107: MS M/Z (ES⁻) 671 (MH⁻, 25), 212 (100).

II. Biological Section

Materials

Heparin Sepharose CL-6B was purchased from Pharmacia (Amersham Pharmacia Biotech, Uppsala, Sweden); 1,9-dimethyl-methylene blue (DMB) and heparan sulfate were purchased from Sigma-Aldrich (Rehovot, Israel); MCDB 131 medium was purchased from Clonetics (San Diego, Calif., USA); DMEM and fetal calf serum were purchased from Gibco BRL (InVitrogen Corporation, CA, USA); glutamine, gentamicin and Hank's balanced salt solution (HBSS) were purchased from Biological Industries (Bet Haemek, Israel). The BD BioCoat Angiogenesis System kit-elements and the BD Oxygen Biosensor System kit-elements were purchased from BD Biosciences (MA, USA); Calcein AM (Cat No C3100) was purchased from Molecular Probes Europe BV (Leiden, The Netherlands). 96-well plates were purchased from Greiner Labortechnik GmbH (Frickenhausen, Germany).

Methods

(a) In Vitro Dimethylmethylene Blue (DMB) Assay for Heparanase Activity

Heparin Sepharose CL-6B beads were added up to the top of the wells of a multiscreen column loader (Millipore). A 96-well multiscreen plate containing 0.65 μm hydrophilic, low protein binding, Durapore membrane (Millipore) was placed, upside down, on top of the multiscreen column loader. The column loader and the multiscreen plate were held together, turned over, and the beads were uniformly transferred from the column loader to the multiscreen plate (Millipore, MADVM 650). Double-distilled water (DDW) was then added to the beads, which were allowed to swell for one minute, and then washed (three times) with DDW under vacuum. Heparin concentration was estimated to be 10 μM/well.

Human recombinant heparanase of at least 50% purity was obtained by expression in the CHO cells S1-11 subclone (generated as described for CHO clones S1PPT-4 and S1PPT-8 in WO 99/57244). Active human recombinant heparanase, purified from the CHO cell extracts by ion exchange chromatography (as described for the CHO 2TT1-8 subclone in WO 99/57244), was added (5 ng/well) to a reaction mixture containing 20 mM phosphate citrate buffer, pH 5.4, 1 mM CaCl₂, 1 mM NaCl. After 3-hour incubation at 37° C. in an incubator on a rotator, the heparanase reaction products were filtered under vacuum and collected into a 96-well polystyrene flat bottom plate (Greiner Cat. No. 655101). To each well, phosphate-buffered saline (PBS) containing 1% bovine serum albumin (BSA; 70 μl/well) and DMB (32 mg of DMB were dissolved in 5 ml ethanol, diluted to 1 liter with formate buffer containing 4 g sodium formate and 4 ml formic acid; 120 μl/well) were added. Color was developed after 5 minutes, and the absorbance of the samples was determined using a microplate reader (Spectra Max, Molecular Devices) at 530 nm with 570 nm as reference. The absorbance correlated to heparanase activity. As a control, heparanase was added to the heparin Sepharose swollen beads in the multiscreen plate and the heparanase reaction products were filtered immediately thereafter and the absorbance of these control samples was subtracted from all other samples.

Alternatively, instead of the partially purified human recombinant heparanase enzyme as above, crude extracts of CHO cells S1-11 subclone expressing human recombinant or crude extracts of CHO cells mhG9 clone expressing mouse recombinant heparanase (generated with the mouse heparanase cDNA as described for CHO clones expressing human recombinant heparanase in WO 99/57244) were used. The cell extracts were centrifuged and resuspended in 20 mM phosphate citrate buffer, pH 5.4 containing 50 mM NaCl. The cells were lysed by three cycles of freezing and thawing. The cell lysates were centrifuged (10000×g for 5 min), supernatants were collected and then assayed for heparanase activity using the DMB assay.

In order to examine whether a test compound exhibits an inhibitory effect on the heparanase activity, each compound was dissolved in dimethylsulfoxide (DMSO) and added, at a concentration range of 1-30 μM, to the heparin Sepharose swollen beads in the 96-multiscreen plate. The partially purified human recombinant heparanase or the crude cell extracts expressing either human or mouse recombinant heparanase were added for a 3-hour incubation and the reaction continued as described above. Absorbance of the developing color was measured as described above. The IC₅₀ value (the concentration at which the heparanase activity was inhibited by 50%) for each compound was evaluated for the relevant range of concentrations according to the preliminary screening results.

(b) Determination of Cytotoxicity of the Compounds

The measurements of cytotoxicity of the tested compounds was based on monitoring the dissolved oxygen concentrations in the medium of cultured cells, using the BD Oxygen Biosensor System kit. The measuring system is based on an oxygen sensitive fluorescent compound [tris (4,7-diphenyl-1,10-phenanthroline) ruthenium (II) chloride] embedded in a hydrophobic matrix, permanently attached to the bottom of a multiwell plate. The oxygen in the vicinity of the dye (which concentration is in equilibrium with that in the liquid media) quenches the dye in a predictable concentration-dependent manner. The amount of fluorescence correlates directly to the rate of oxygen consumption in the well, which in turn is related to cell viability and growth.

The compounds tested for cytotoxicity were dissolved in DMSO and diluted to give final concentrations of IC₅₀×2000, IC₅₀×1000, and IC₅₀×200. 200 μl of cells (human sarcoma HT1080 cells, final concentration 1.5×10⁵ cell/ml) suspended in DMEM were transferred to a polypropylene u-bottom 96-well plate, together with 2 μl of each inhibitor solution or DMSO (serving as control). The plates were incubated for 22 hours at 37° C. in an 8% CO₂ atmosphere. Cell viability in the presence of the tested compounds was assessed by monitoring the fluorescence in each well (fluorescence parameters: excitation 485 mn, emission 590 nm, POLARstar Galaxy Fluorometer). High fluorescent signals correlated with high oxygen consumption by the cells, indicating high cell viability and growth, whereas a decrease in signal intensity was indicative of a decrease in oxygen consumption and, therefore, loss of cell viability.

(c) In Vitro Assay of Invasion Inhibition by Heparanase Inhibitors

The ability of the compounds of the invention to inhibit cell invasion was determined quantitatively by the in vitro Endothelial Cell Migration assay using a BD BioCoat Angiogenesis System kit. The kit consists of a 24-multiwell insert plate (FluoroBlok, BD Falcon) containing a microporous (3.0 μm pore size) polyethylene terephthalate (PET) membrane that is capable of blocking fluorescence completely (>99% efficiency). This membrane is uniformly coated with matrigel (BD Matrigel Matrix). The uniform layer of matrigel matrix serves as a reconstituted authentic basement membrane in vitro, providing a true barrier to non-invasive cells, but allowing endothelial cells to attach to the membrane and freely migrate towards an angiogenic stimulus in the lower chamber of the insert plate. Post-labeling the cells with a fluorescent dye and measuring the fluorescence of invading cells in a fluorescent plate reader, provides quantitative measurement of cell invasion.

Each of the tested compounds was diluted to a concentration that was found to be non-toxic to the HT1080 cells, according to the toxicity assay described in (b) above. To cover the optimal seeding density for HT1080 cells, suspensions containing various cell concentrations were prepared: 1 ml of 3×10⁵ cells/ml, 8 ml of 1.5×10⁵ cells/ml and 1 ml of 0.75×10⁵ cells/ml. The top chambers of each well in the inserts was filled with 0.25 ml cell-suspension, 750 μM DMEM containing 5% fetal calf serum and an inhibitor solution. The plates were incubated for 22 hours at 37° C. and 8% CO₂ atmosphere. At the end of incubation, the medium was aspirated from the upper chambers, and the insert was transferred into a second 24-well plate containing 0.5 ml/well of the fluorescent dye Calcein AM solution (4 μg/ml per plate, prepared from 50 μg Calcein AM dissolved in 20 μl DMSO and 12.5 ml of warm HBSS medium), and incubated for 90 minutes at 37° C., 8% CO₂ atmosphere. Fluorescence of invaded cells was read in a fluorescence plate reader with bottom read capabilities at excitation/emission wavelength of 485/530 nm, without further manipulation. Only those labeled cells that have invaded the matrigel and passed through the pores of the PET membrane, were detected. Since the fluorescent blocking membrane effectively blocked the passage of light from 490-700 nm, fluorescence from cells that have not invaded the membrane was blocked from detection (POLARstar, Galaxy).

(d) In Vivo Mouse Melanoma Primary Tumor Growth Assay for Heparanase Activity

Instead of using a primary tumor cell line, primary tumor was generated in C57BL mice by cells herein designated FOR cells, which were generated as follows: B16-F1 mouse melanoma cells (ATCC No. 6326) were grown in DMEM containing 10% fetal calf serum, 2 mM glutamine, and 50 μg/ml gentamicin. A subclone of the B16-F1 cell line, F1-J, produced large amounts of melanin and exhibited a highly metastasis potential. These highly metastatic F1-J cells were injected to syngeneic mice (100,000 cells, s.c.). Cells from metastases that were formed were cultured in different conditions. A clone, F1-LG, designated herein FOR, was selected by its high heparanase expression and activity using the reverse transcriptase-polymerase chain reaction (RT-PCR) and the radiolabeled ECM degradation analyses, respectively, as previously described (Vlodavsky et al., 1999; U.S. 6,190,875).

FOR cells were grown in DMEM containing 10% fetal calf serum, 2 mM glutamine, and 50 μg/ml gentamicin until they reached confluence (typically 4-5 days) and then splitted (1:5). This splitting yielded subconfluent and growing cells at day 7, the day of cell injection, at which the cells were trypsinized, washed with PBS and counted to yield a cell suspension of 10⁶ cells/mil in PBS. Male C57BL mice (˜20 gr each; at least 10 mice/group) were injected s.c. on the flank with a suspension of the FOR cells (100 μl/mouse). Four days later, a test compound dissolved in DMSO was injected (100 μl) i.p to the mice, twice a day (morning and evening). Each compound was injected at either 1 or 2 different concentrations (0.1 and/or 0.5 mg/mouse/day). Control mice were injected i.p. with DMSO only (100 μl). Mice were observed daily, and usually three weeks after cell injection, mice were sacrificed, the tumors were harvested and weighted.

(e) Transmigration Assay for Heparanase Activity

An in vitro chamber-like transmigration system was established by using transwell filters coated with a reconstituted basement membrane-like matrix (matrigel). Matrigel is composed of laminin, collagen type IV, entactin and nidogen, as well as of HSPG, thus constituting a relevant heparanase substrate. The cells used in the experiment were mock-transfected Eb murine lymphoma cells not expressing heparanase and stable hepa-transfected Eb murine lymphoma cells overexpressing heparanase (both cells described by Vlodavsky et al., 1999), and the migration rate of the cells trough Matrigel was evaluated first in the absence and in the presence of the chemoattractant SDF-1. Once the transmigration of the cells to the lower chamber was shown to be well correlated with the heparanase expression levels and activity, the transmigration of the Eb cells overexpressing heparanase was tested after treatment with the heparanase inhibitors of the invention. Addition of the heparanase inhibitor reduces the transmigration rate of the cells.

### Example 23

### Biological Activity of the Compounds 1-107

Compounds 1-107 were tested according to one or more of the assays described in (a)-(e) above. Results of the IC₅₀ values of the different compounds are shown in Appendix A. All tested compounds were found to inhibit heparanase activity at micromolar and submicromolar concentrations. Some compounds such as Compounds 1, 2, 3 and others were found to be effective inhibitors of cell invasion (“yes” in right column of the table depicted in Appendix A).

### REFERENCES

Courtney, S. M., Hay, P. A., Buck, R. T., Colville, C. S., Porter, D. W., Scopes, D. I., Pollard, F. C., Page, M. J., Bennett, J. M., Hircock, M. L,. McKenzie, E. A., Stubberfield, C. R and Turner, P. R. (2004) 2,3-Dihydro-1,3-dioxo-1H-isoindole-5-carboxylic acid derivatives: a novel class of small molecule heparanase inhibitors. Bioorg. Med. Chem. Lett. 14(12):3269-73.

Eastmond, G. C. and Paprotny, J. (1998) Methyl- and fluoro-substituted bis(4-aminophenoxy) benzenes. A convenient method of synthesis. Synthesis, 6: 894-898.

Elkin M, Ilan N, Ishai-Michaeli R, Friedmann Y, Papo O, Pecker I. and Vlodavsky I. (2001) Heparanase as mediator of angiogenesis: mode of action. FASEB J. 15(9):1661-3.

Ferro, V., Hammond, E. and Fairweather, J. K. (2004) The development of inhibitors of heparanase, a key enzyme involved in tumor metastasis, angiogenesis and inflammation. Mini Rev. Med. Chem. 4(6):693-702.

Freeman, C., Liu, L., Banwell, M. G., Brown, K. J., Bezos, A., Ferro, V. and Parish, C. R. (2005) Use of sulfated linked cyclitols as heparan sulfate mimetics to probe the heparin/heparan sulfate binding specificity of proteins. J. Biol. Chem. Jan 4 on Internet.

Ishida, K., Hirai, G., Murakami, K., Teruya, T., Simizu, S., Sodeoka, M. and Osada, H. (2004) Structure-based design of a selective heparanase inhibitor as an antimetastatic agent. Mol. Cancer Ther. 3(9):1069-77.

Ishida, K., Teruya, T., Simizu, S. and Osada, H. (2004) Exploitation of heparanase inhibitors from microbial metabolites using an efficient visual screening system. J Antibiot. (Tokyo). 57(2):136-42.

Kawase, Y., Takahashi, M., Takatsu, T., Arai, M., Nakajima, M. and Tanzawa, K. (1995) A-72363 A-1,A-2, and C, novel heparanase inhibitors from Streptomyces nobilis SANK 60192. II. Biological activities. J. Antibiotics 49: 61-64.

Ko, H. R., Kim, B. Y., Oh, W. K., Kang, D. O., Lee, H. S., Koshino, H., Osada, H., Mheen, T. I. and Ahn J. S. (2000) CRM646-A and -B, novel fungal metabolites that inhibit heparinase. J Antibiot (tokyo) 53:211-4.

Lapierre, F., Holme, K., Lam, L., Tressler, R. J., Storm, N., Wee, J., Stack, R. J., Casrellot, J. and Tyrrell, D. J. (1996) Chemical modifications of heparin that diminish its anticoagulant but preserve its heparanase-inhibitory, angiostatic, anti-tumor and anti-metastatic properties. Glycobiol. 6: 355-366.

Lider, O., Baharav, E., Mekori, Y. A., Miller, T., Naparstek, Y., Vlodavsky, I. and Cohen, I. R. (1989) Suppression of experimental autoimmune diseases and prolongation of allograft survival by treatment of animals with heparinoid inhibitors of T lymphocyte heparanase. J. Clin. Invest. 83: 752-756.

Naggi, A., Casu, B., Perez, M., Torri, G., Cassinelli, G., Penco, S., Pisano, C., Giannini, G., Ishai-Michaeli, R. and Vlodavsky I. (2005) Modulation of the heparanase-inhibiting activity of heparin through selective desulfation, graded N-acetylation, and glycol-splitting. J Biol. Chem. Jan 12 on Internet.

Nakajima, M., DeChavigny A., Jolmson, C. E., Hamada, J-I, Stein, C. A. and Nicolson, G. L. (1991) Suramin a potent inhibitor of melanoma heparanase and invasion. J. Biol. Chem. 266: 9661-9666.

Nakajima, M., Irimura, T. and Nicolson, G. L. (1988) Heparanase and tumor metastasis. J. Cell. Biochem. 36: 157-167.

Nakajima, M., Irimura, T., Di Ferrante, N. and Nicolson, G. L (1984) Metastatic melanoma cell heparanase. Characterization of heparan sulfate degradation fragments produced by B16 melanoma endoglucuronidase J. Biol. Chem. 259: 2283-2290.

Nicosia, R. F., Lin, Y. J., Hazelton, D. and Qian, X. (1997) Endogenous regulation of angiogenesis in the rat aorta model. Amer. J. Pathol. 151: 1379-1386.

Nicosia, R. F. and Ottinetti, A. (1990) Growth of microvessels in serum-free matrix culture of rat aorta: a quantitative assay of angiogenesis in vitro. Lab. Invest. 63: 115-122.

Nishimura, Y., Kudo, T., Kondo, S., Takeuchi, T., Tsuruoka, T., Fukuyasu, H. and Shibahara, S. (1994) Totally synthetic analogs of siastatin B. III. Trifluoroacetamide analogs having inhibitory activity for tumor metastasis. J. Antibiot. 47: 101-107.

Parish, C. R., Coombe, D. R., Jackson, K. B. and Underwood P. A. (1987) Evidence that sulfated polysaccharides inhibit tumor metastasis by blocking tumor cell-derived heparanase. Int. J. Cancer 40: 511-517.

Parish, C. R., Freeman, C., Brown, K. J., Francis, D. J. and Cowden, W. B. (1999) Identification of sulfated oligosaccharide-based inhibitors of tumor growth and metastasis using novel in vitro assays for angiogenesis and heparanase activity. Cencer Res. 59: 3433-3441.

Shevelev, S. A., Dutov, M. D., Vatsadze, I. A., Serushkina, O. V., Korelev, M. A. and Rusanov, A. L. (1995) Phenol substitution of nitro groups in 1,3,5-trinitrobenzene—method of preparation of 5-nitroresorcinol diaryl ethers and 3,5-dinitrophenyl aryl ethers. Izv. Akad. Nauk, Ser. Khim. 2: 393-394.

Shiozawa, H., Takahashi, M., Takatsu, T., Kinoshita, T., Tanzawa, K., Hosoya, T., Furuya, K., Takahashi, S., Furihata, K. and Seto, H. (1995) Trachyspic acid, a new metabolite produced by Talaromyces trachyspermus, that inhibits tumor cell heparanase: taxonomy of the producing strain, fermentation, isolation, structural elucidation, and biological activity. J Antibiot (Tokyo). 48:357-62.

Vlodavsky, I., Friedmann, Y., Elkin, M., Aingorn, H., Atzmon, R., Ishai-Michaeli, R., Bitan, M., Pappo, O., Peretz, T., Michal, I., Spector, L. and Pecker, I. (1999) Mammalian heparanase: Gene cloning, expression and function in tumor progression and metastasis. Nat. Med. 5: 793-802.

Vlodavsky, I., Hua-Quan Miao., Benezra, M., Lider, O., Bar-Shavit, R., Schmidt, A. and Peretz, T. (1997) Involvement of the extracellular matrix, heparan sulfate proteoglycans and heparan sulfate degrading enzymes in angiogenesis and metastasis. In: Tumor Angiogenesis. Eds. C. E. Lewis, R. Bicknell & N. Ferrara. Oxford University Press, Oxford UK, pp. 125-140.

Vlodavsky, I., Mohsen, M., Lider, O., Svahn, C. M., Ekre, H. P., Vigoda, M., Ishai-Michaeli, R. and Peretz, T. (1994) Inhibition of tumor metastasis by heparanase inhibiting species of heparin. Invasion Metastasis 14:290-302.

Vlodavsky, I., Eldor, A., Haimovitz-Freidman, A., Matzner, Y., Ishai-Michaeli, R., Levi, E., Bashkin, P., Lider, O. Naparstek, Y., Cohen, I. R. and Fuks, Z. (1992) Expression of heparanase by platelets and circulating cells of the immune system: Possible involvement in diapedesis and extravasation. Invasion Metastasis 12: 112-127.

Vlodavsky, I., Ishai-Michaeli, R., Bar-Ner, M., Freidman, R., Horowitz, A. T., Fuks, Z. and Biran, S. (1988) Involvement of heparanase in tumor metastasis and angiogenesis. Isr. J. Med. 24: 464-470.

Vlodavsky, I., Fuks, Z., Bar-Ner, M., Ariav, Y. and Schirrmacher, V. (1983) Lymphoma cell mediated degradation of sulfated proteoglycans in the subendothelial extracellular matrix: Relationship to tumor cell metastasis. Cancer Res. 43: 2704-2711.

| COMPOUNDS | CAS No | DMB (h-hpa) IC50[μM] | Invasion |
|---|---|---|---|
| 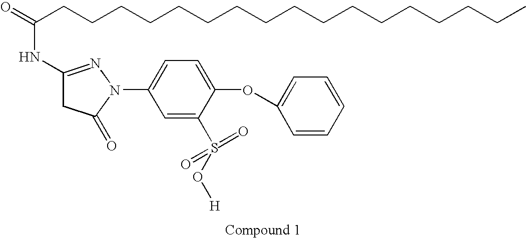 | 125677-03-4 | 0.28 | yes |
| 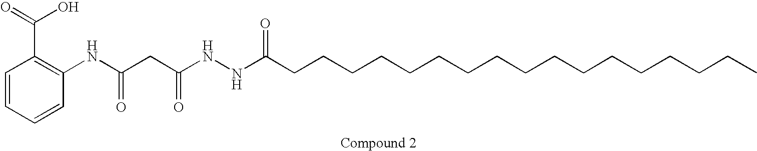 | 319489-50-4 | 1.70 | yes |
| 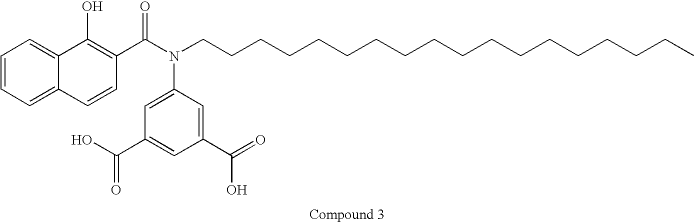 | 119712-98-0 | 0.38 | yes |
| 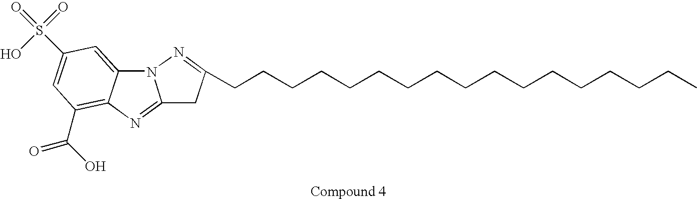 | 292841-33-9 | 1.20 | yes |
| 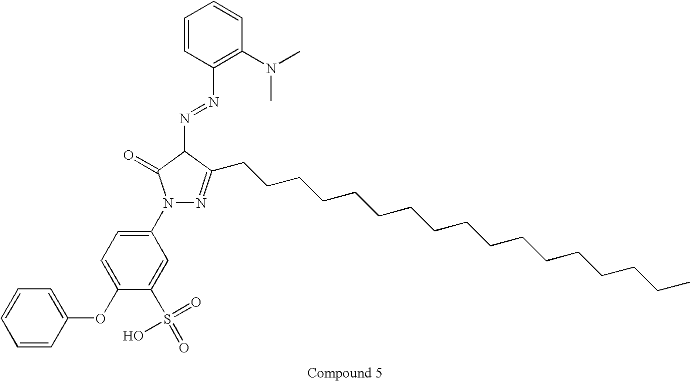 | 96765-80-9 | 0.22 | |
| 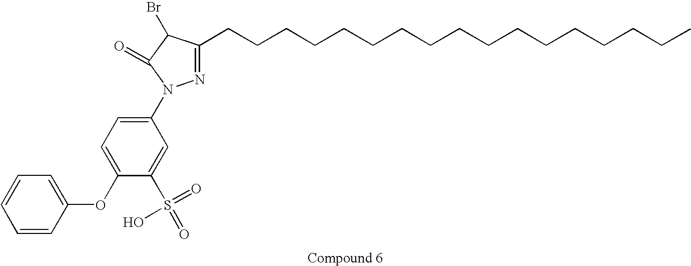 | 439091-52-8 | 0.25 | |
| 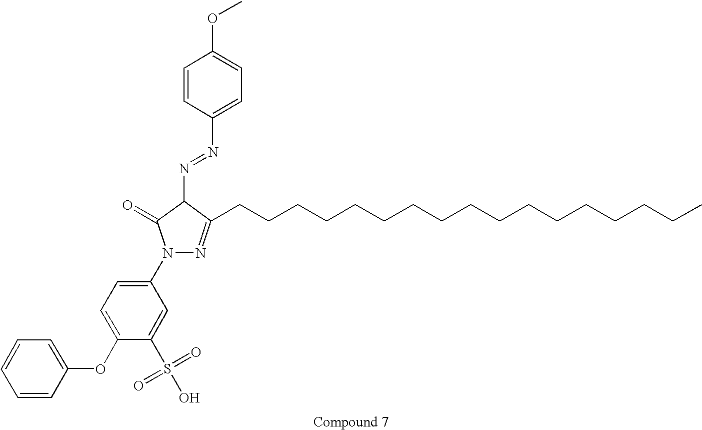 | 325856-62-0 | 0.24 | |
| 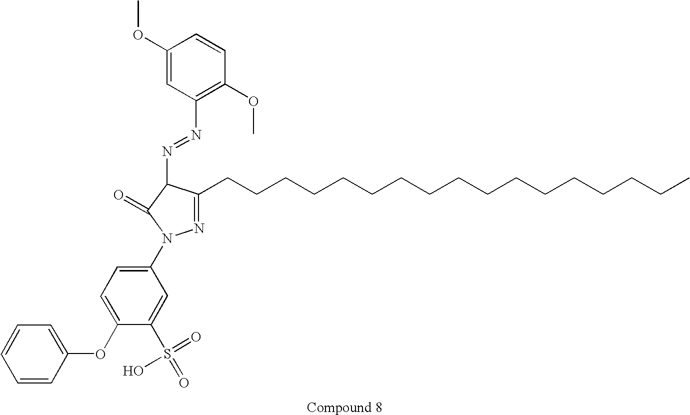 | 97734-03-7 | 0.21 | |
| 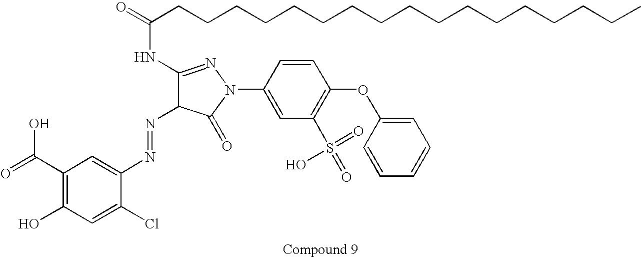 | 55302-60-8 | 0.16 | yes |
| 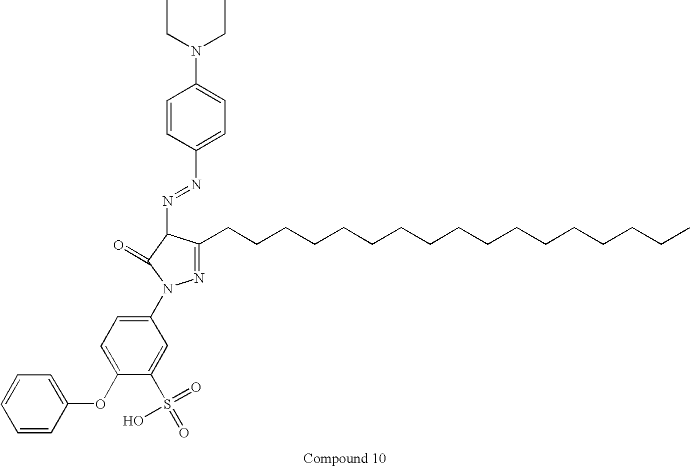 | 31042-44-1 | 0.26 | yes |
| 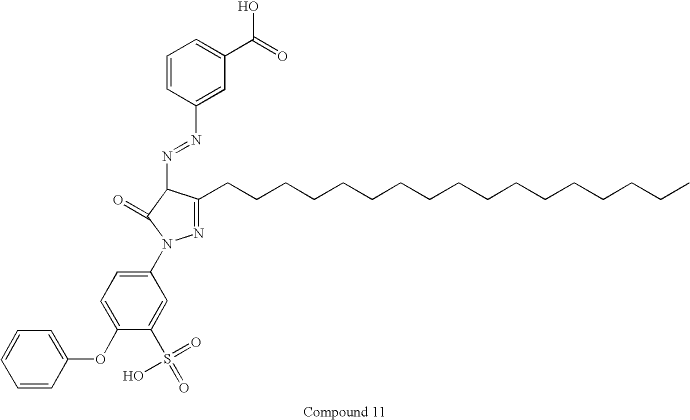 | 478250-38-3 | 0.18 | |
| 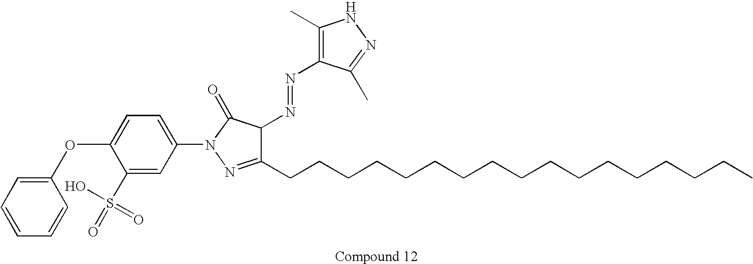 | | 0.24 | yes |
| 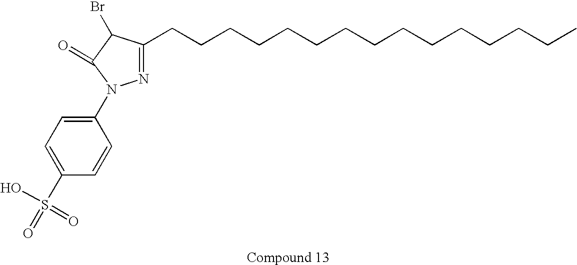 | 438540-22-3 | 1.00 | yes |
| 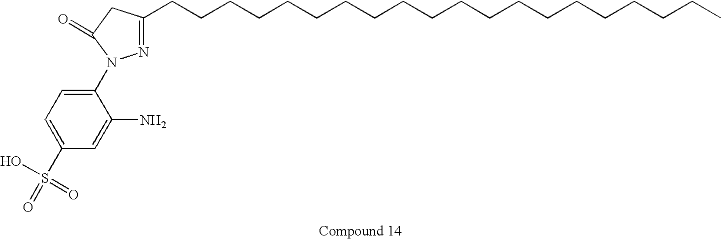 | 96173-75-0 | 0.87 | |
| 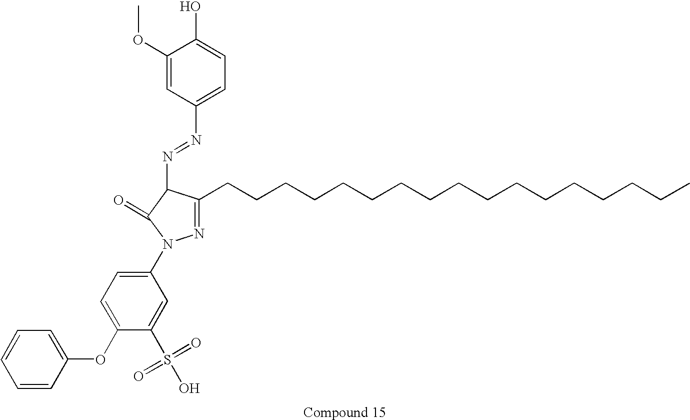 | 97734-12-8 | 0.25 | yes |
| 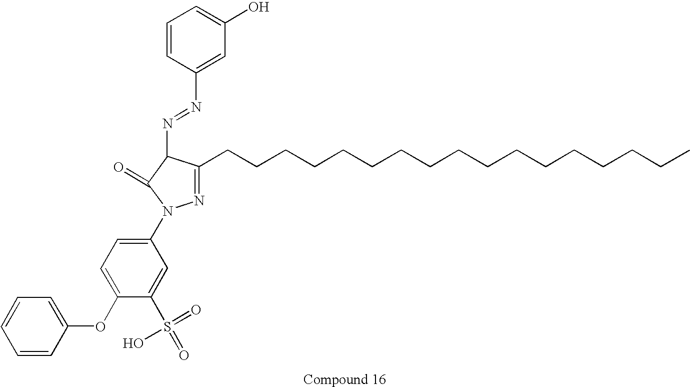 | 97734-03-7 | 0.22 | yes |
| 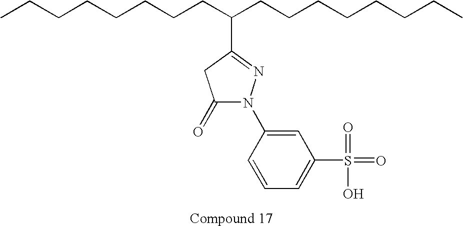 | 293762-31-9 | 2.70 | yes |
| 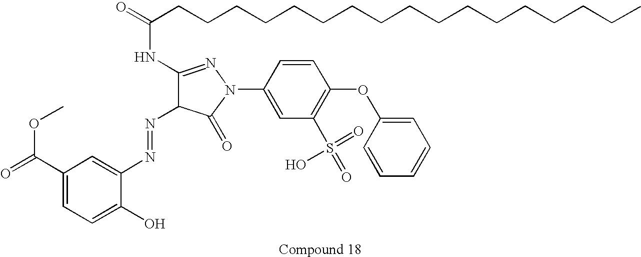 | | 0.29 | yes |
| 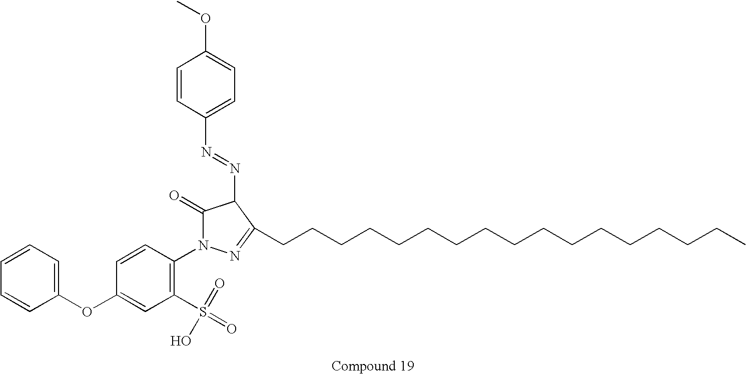 | 325856-62-0 | 0.32 | yes |
| 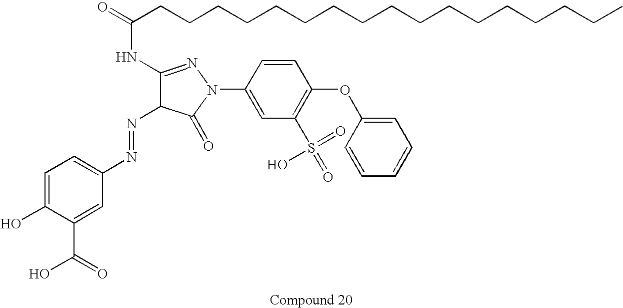 | 55302-59-5 | 0.19 | |
| 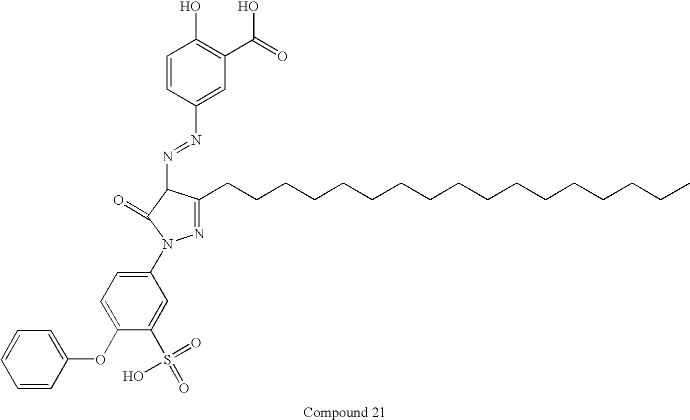 | 96772-14-4 | 0.31 | |
| 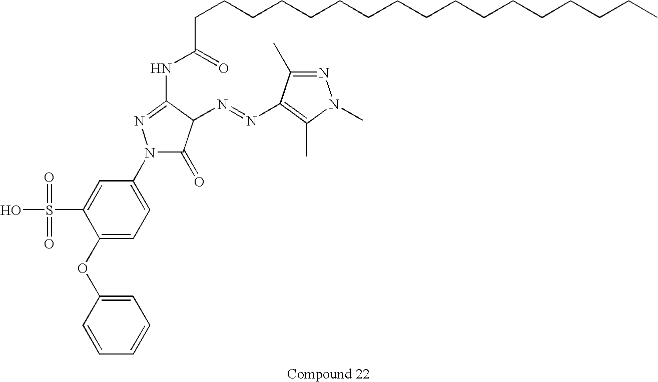 | 293325-35-6 | 0.65 | yes |
| 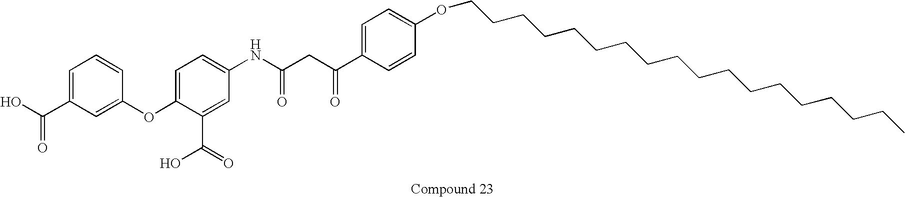 | 17725-26-7 | 0.40 | |
| 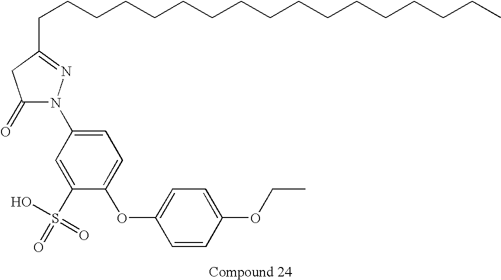 | 293760-73-3 | 0.63 | yes |
| 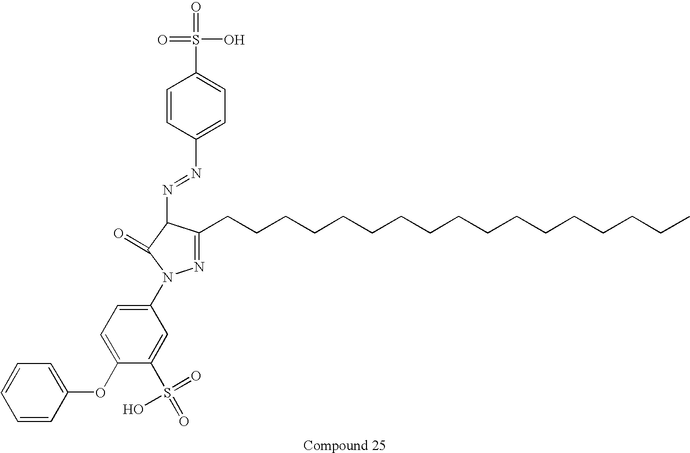 | 21528-59-6 | 0.27 | |
| 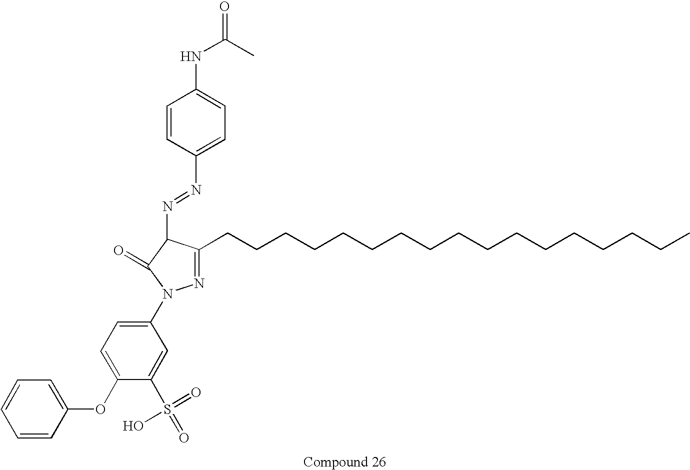 | 97734-34-4 | 0.13 | |
| 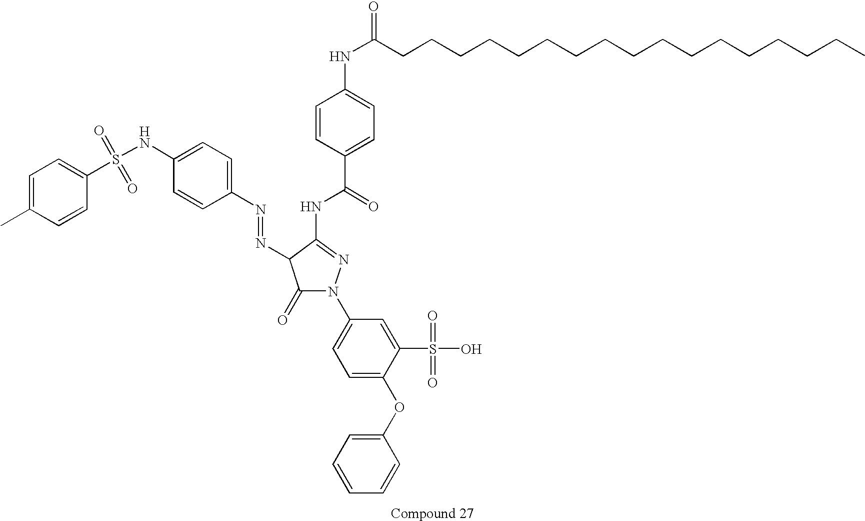 | | 0.27 | |
| 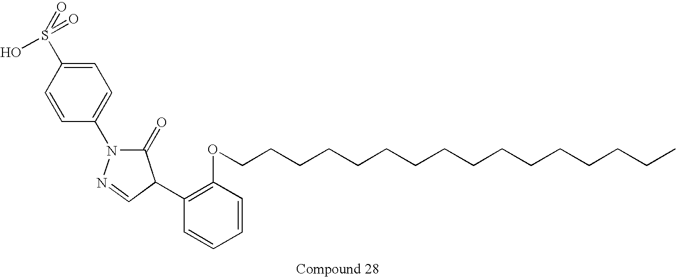 | 198971-79-8 | 1.00 | yes |
| 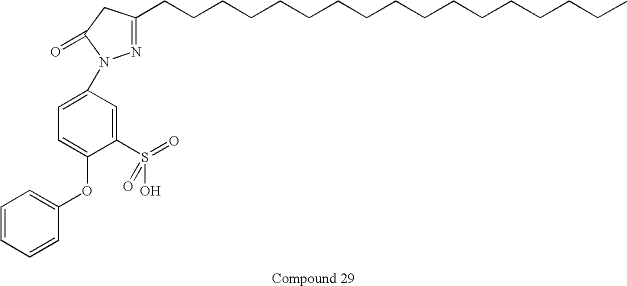 | 13048-16-3 | 0.32 | yes |
| 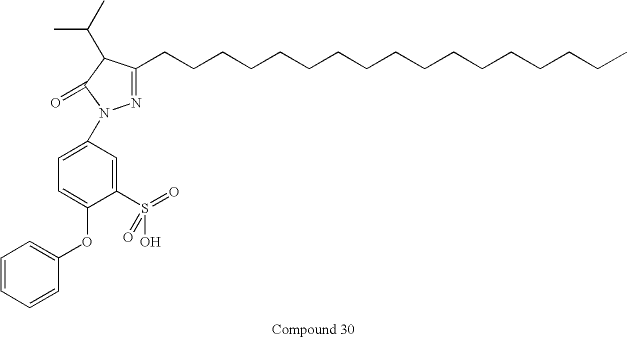 | 115345-38-5 | 0.16 | yes |
| 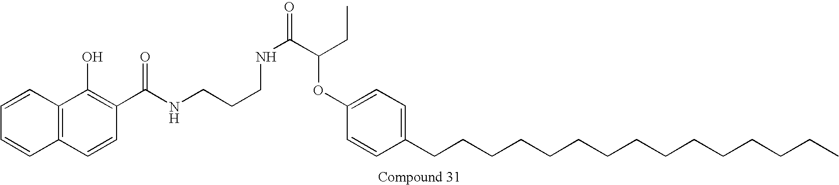 | 355152-86-2 | 0.52 | yes |
| 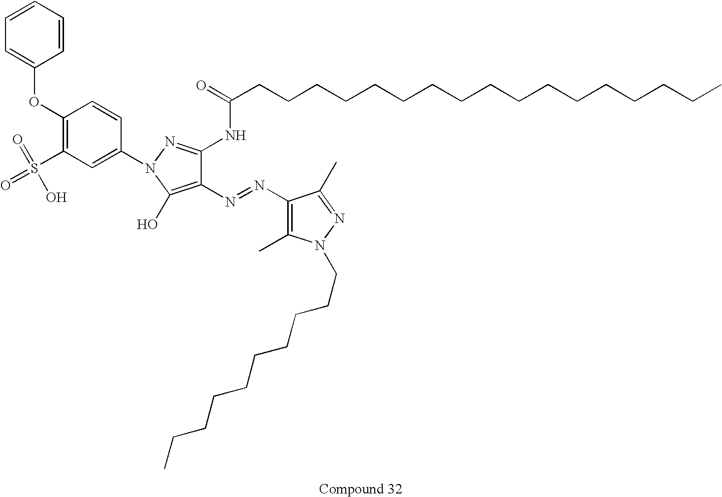 | 292842-85-4 | 0.24 | yes |
| 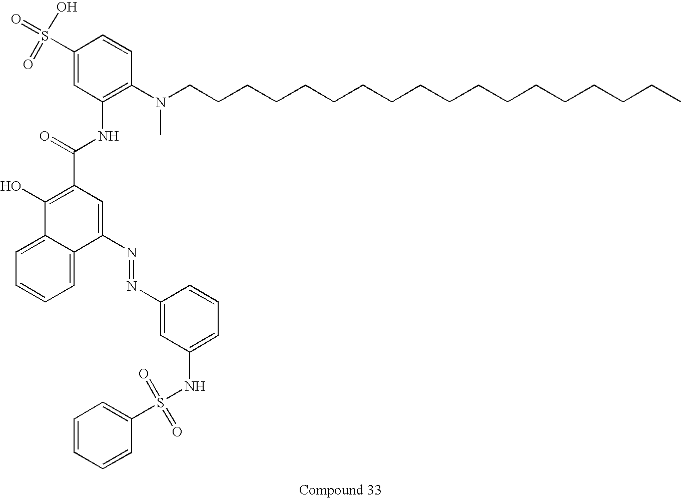 | 10285-76-4 | 0.21 | |
| 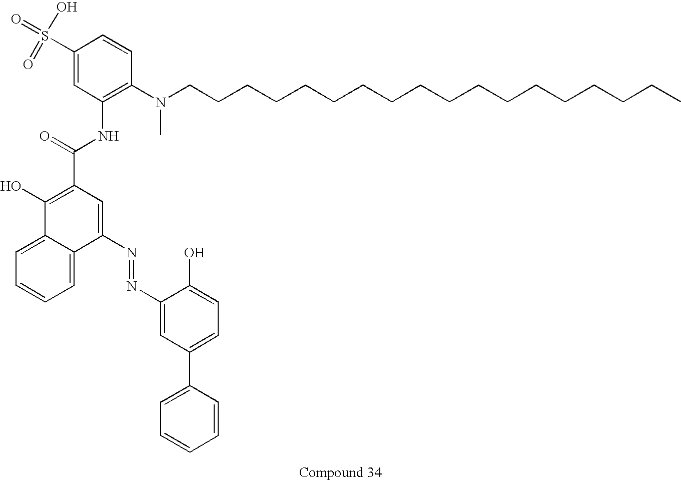 | 478250-30-5 | 0.31 | |
| 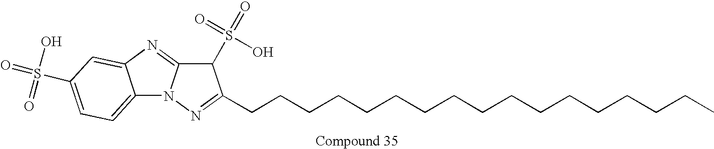 | 33622-69-4 | 1.34 | |
| 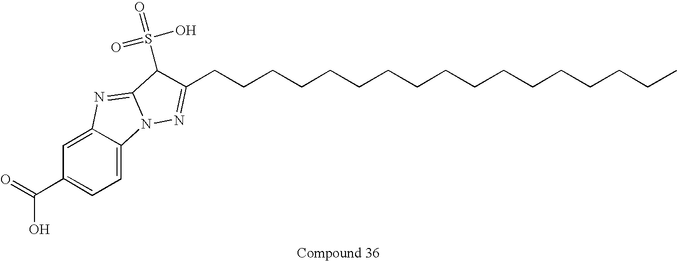 | 292841-33-9 | 1.42 | |
| 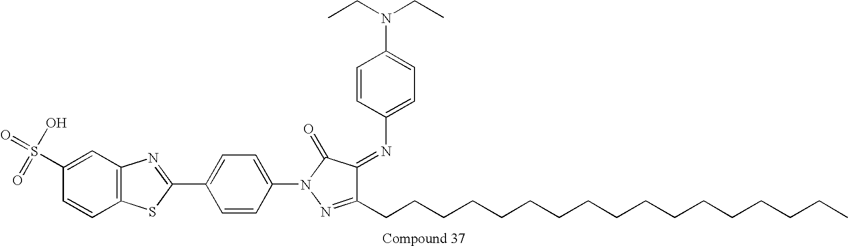 | | 1.50 | |
| 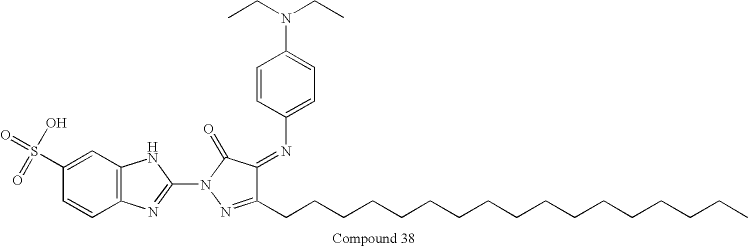 | 122335-06-2 | 1.45 | |
| 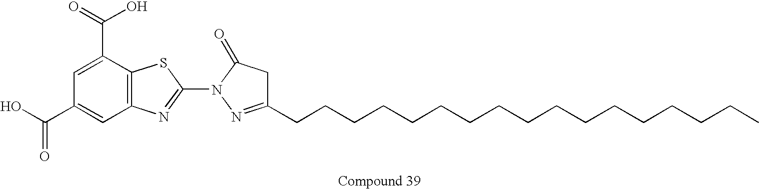 | 116030-46-7 | 2.80 | |
| 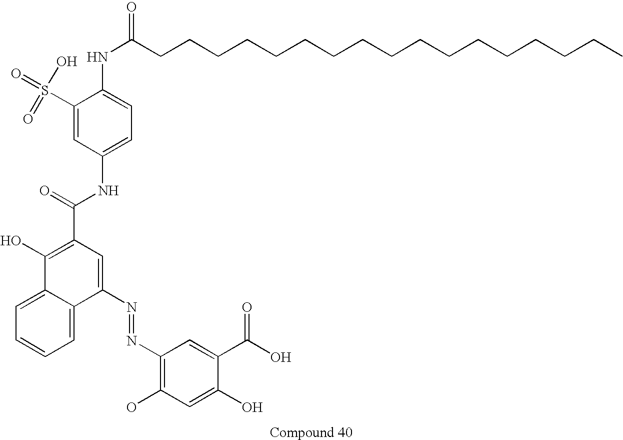 | 55303-50-9 | 0.23 | |
| 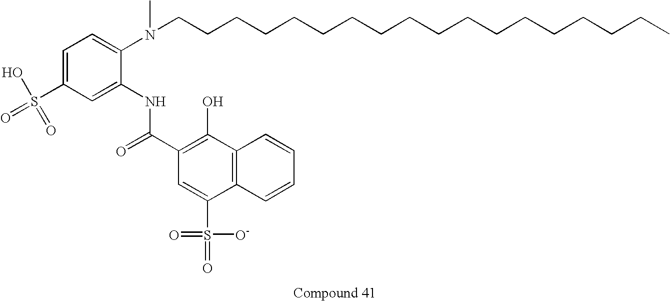 | 133898-04-1 | 0.49 | |
| 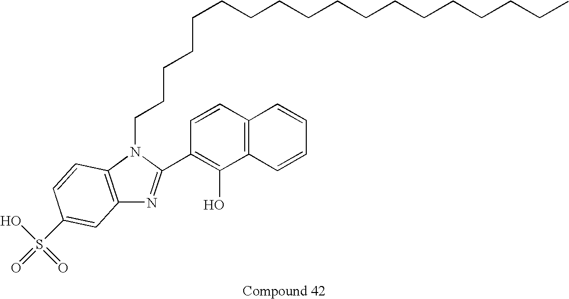 | 96711-28-3 | 0.48 | |
| 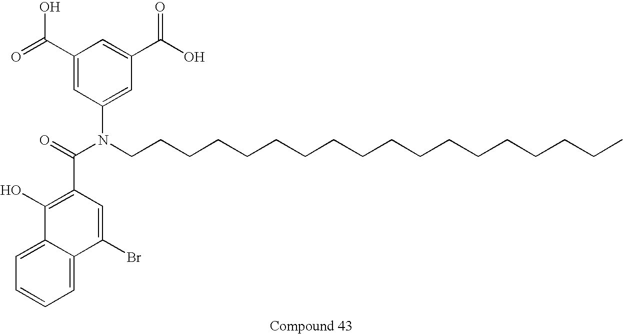 | 438538-56-8 | 0.55 | yes |
| 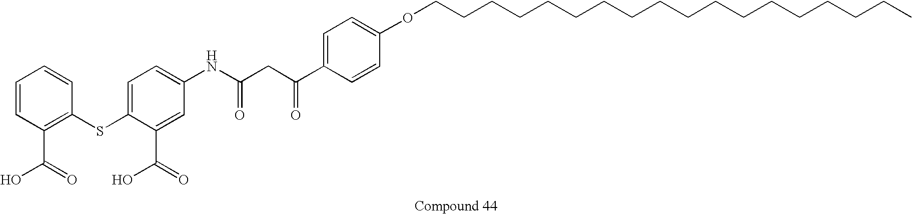 | 293762-35-3 | 0.21 | |
| 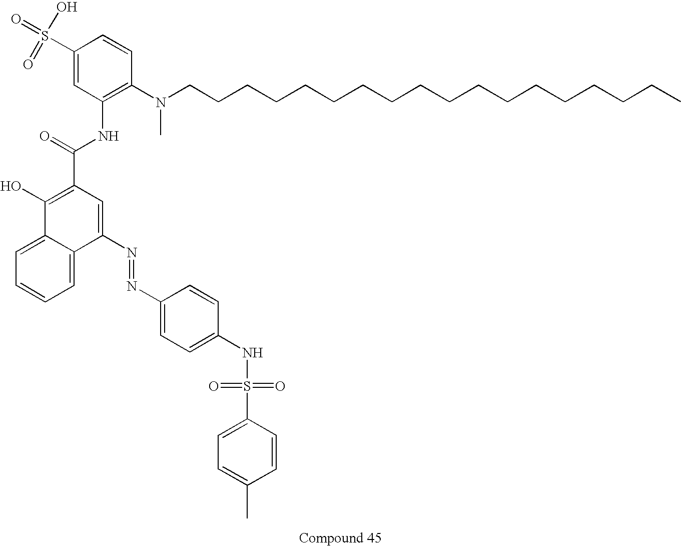 | 10285-76-4 | 0.50 | |
| 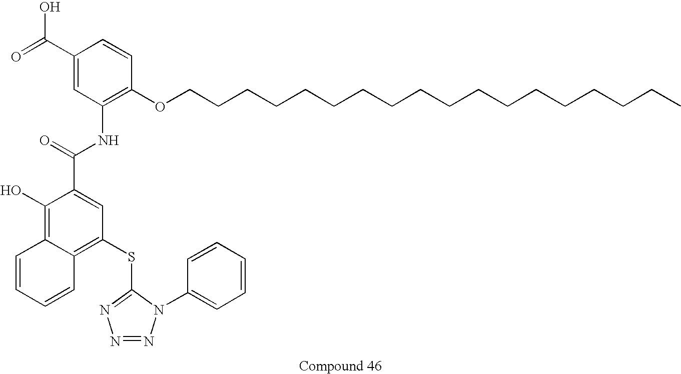 | 81451-93-6 | 7.70 | |
| 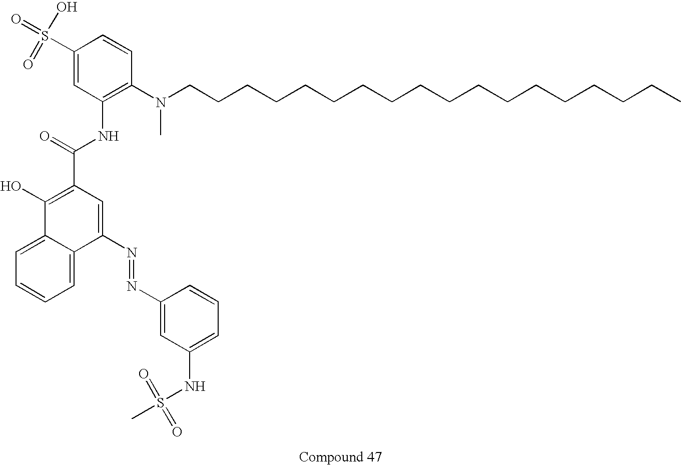 | 40442-59-9 | 0.25 | |
| 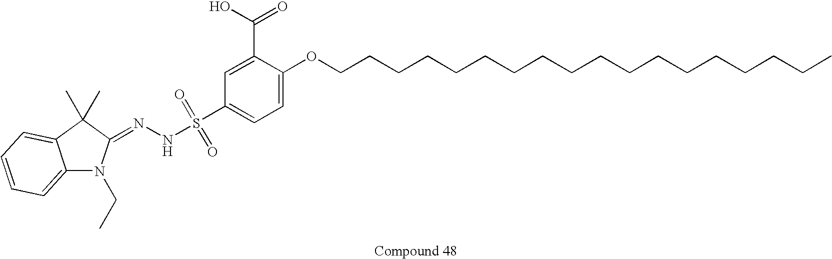 | | 0.95 | |
| 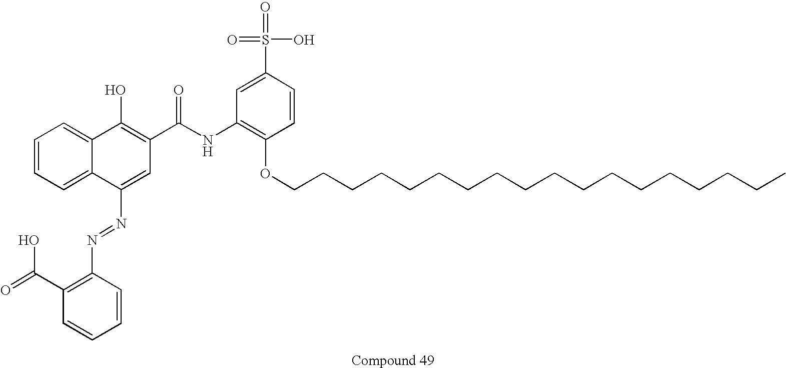 | 6674-98-2 | 2.70 | |
| 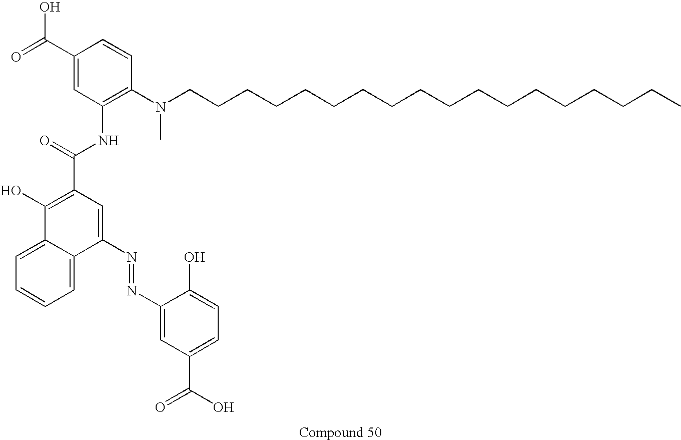 | | 4.50 | |
| 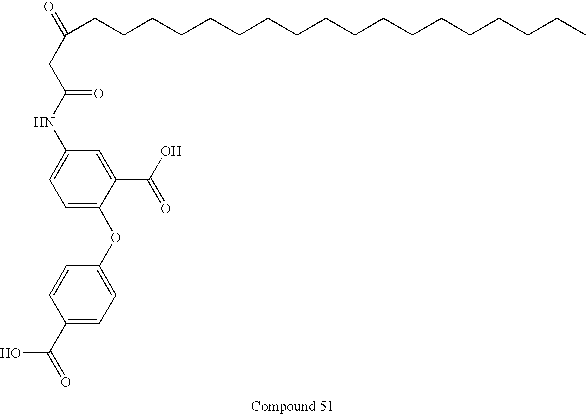 | 293327-38-5 | 3.00 | |
| 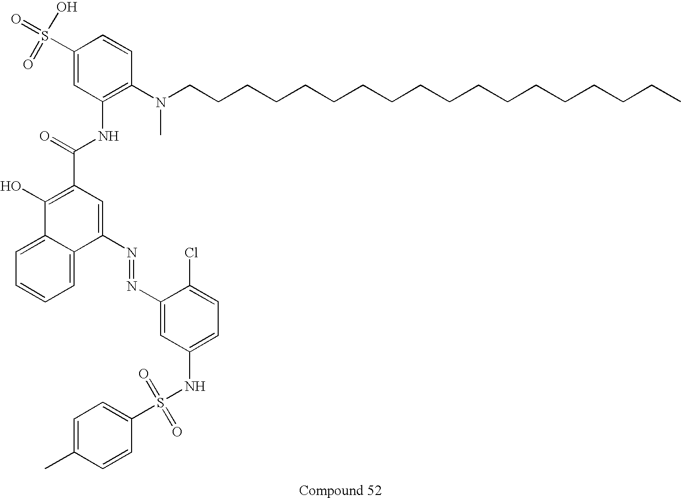 | 478250-29-2 | 0.74 | |
| 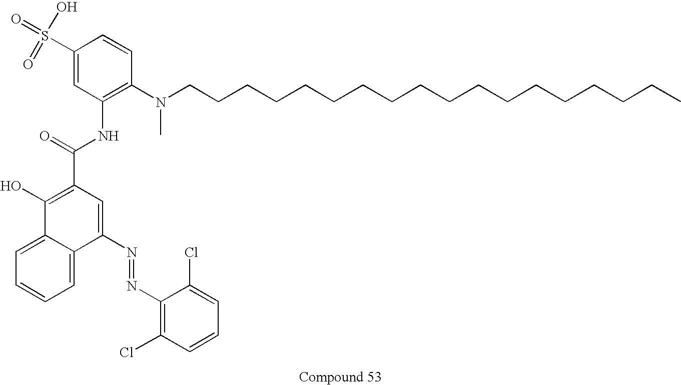 | 105862-69-9 | 0.40 | |
| 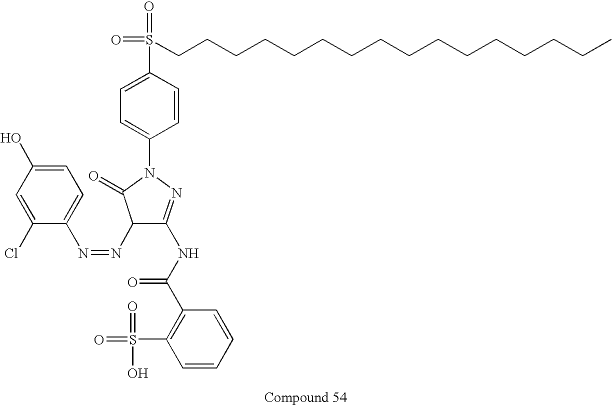 | 153908-56-6 | 0.20 | |
| 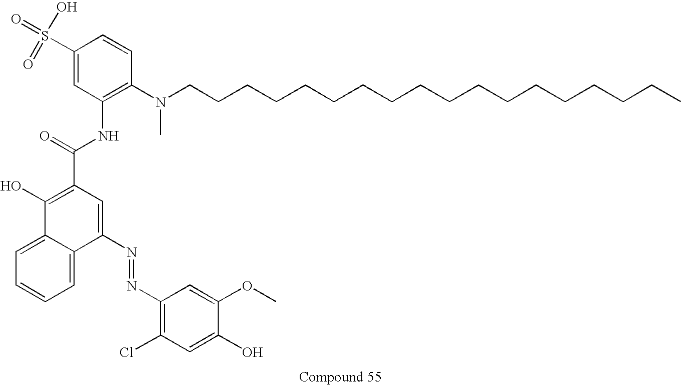 | 55303-50-9 | 0.43 | |
| 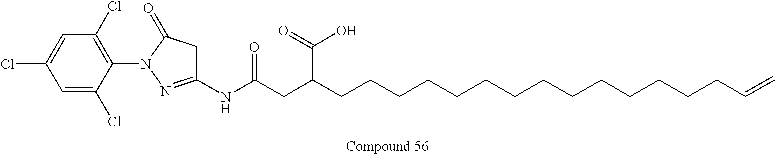 | 353783-36-5 | 6.80 | |
| 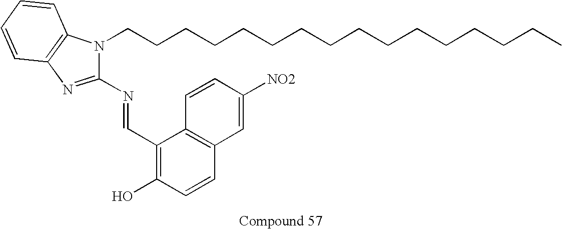 | 342592-08-9 | 7.80 | |
| 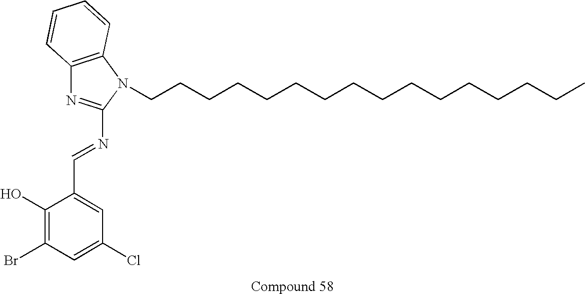 | 343589-13-9 | 3.00 | |
| 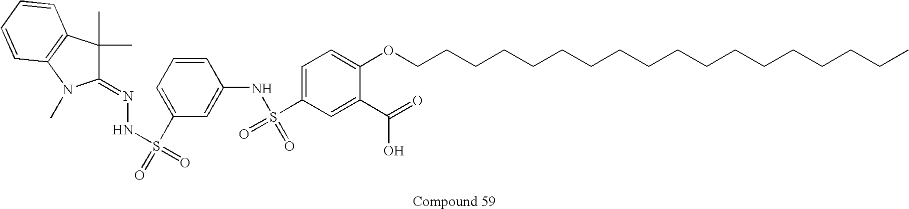 | 342388-95-8 | 0.49 | |
| 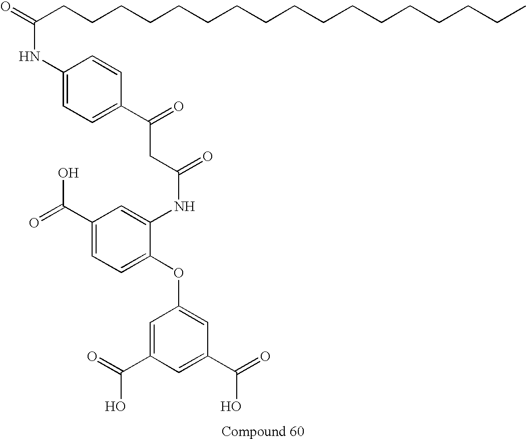 | 17725-27-8 | 0.44 | |
| 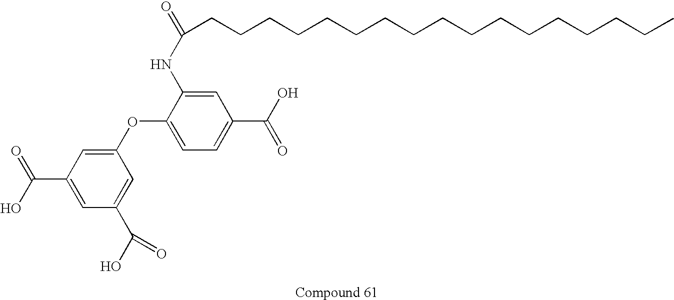 | | 2.00 | |
| 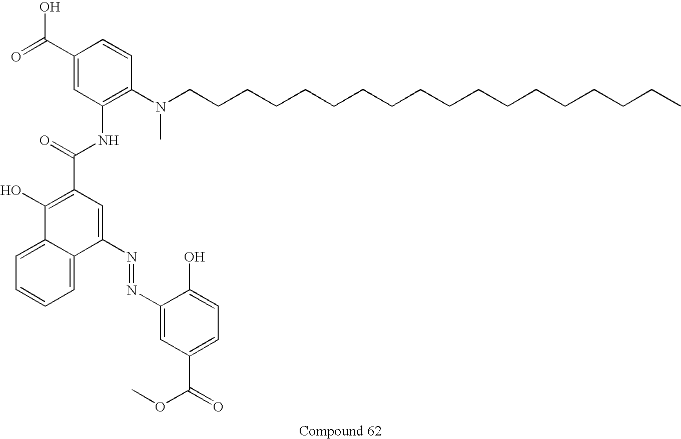 | | 11.64 | |
| 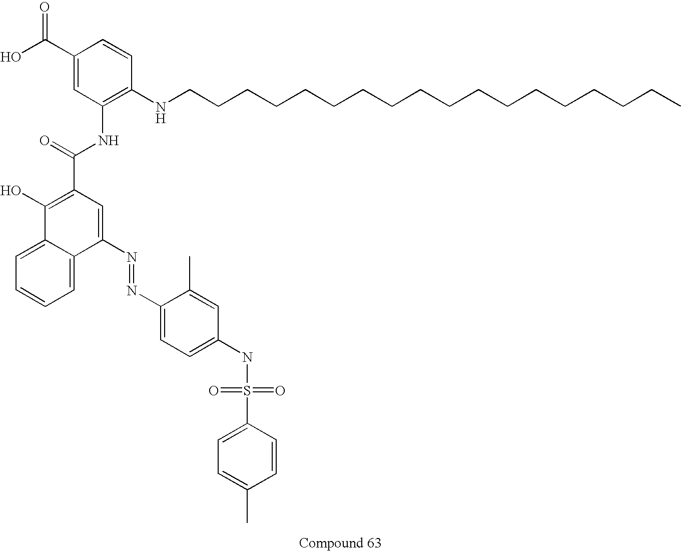 | | 16.83 | |
| 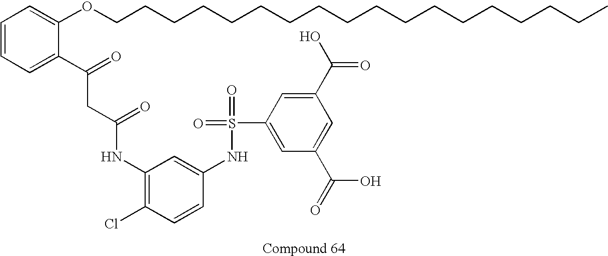 | 70745-82-3 | 0.29 | |
| 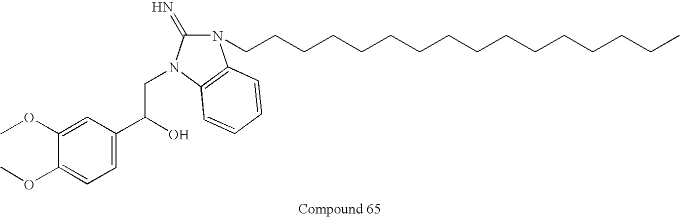 | 487007-26-1 | 4.80 | |
| 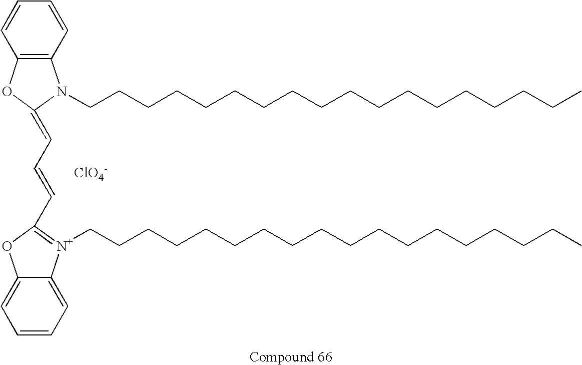 | 34215-57-1 | 1.00 | |
| 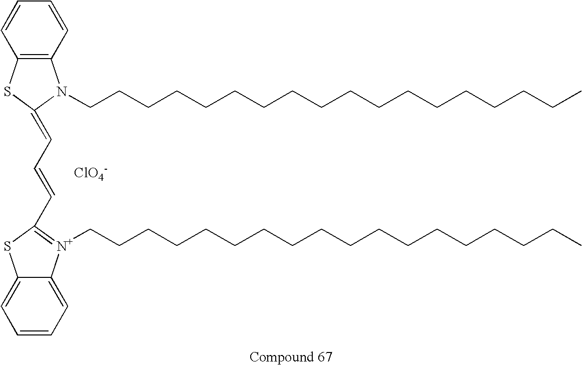 | 53533-50-9 | 1.50 | |
| 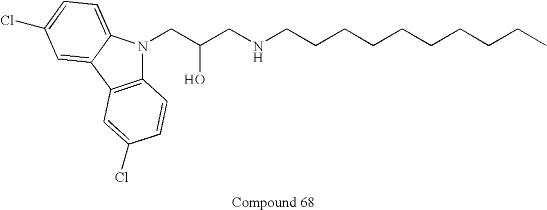 | 488796-23-2 | 6.80 | |
| 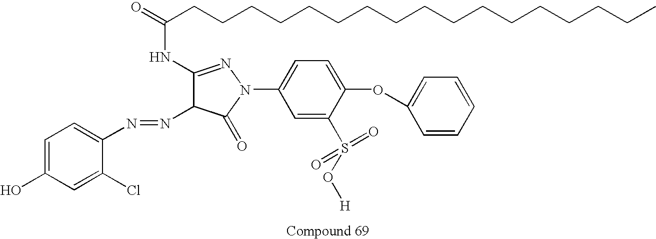 | 30515-97-0 | 0.18 | |
| 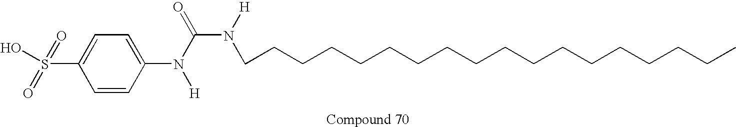 | | 7.00 | |
| 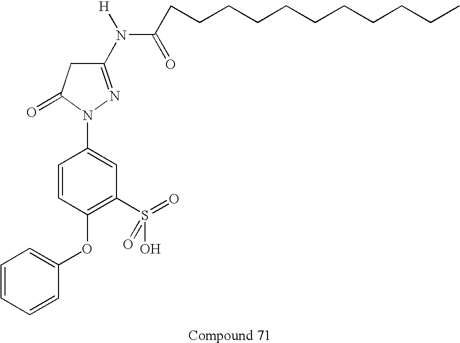 | | 2.65 | |
| 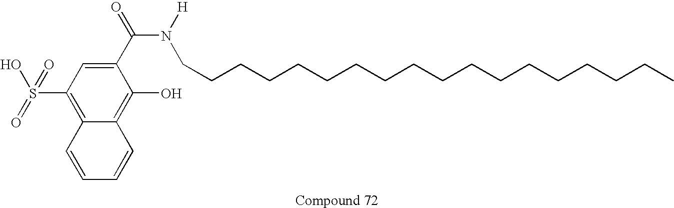 | 2467-29-0 | 0.32 | |
| 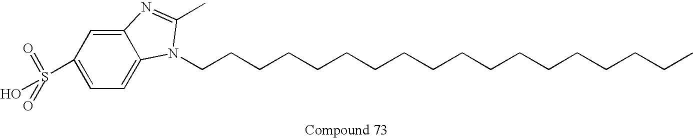 | 200348-21-6 | 1.40 | |
| 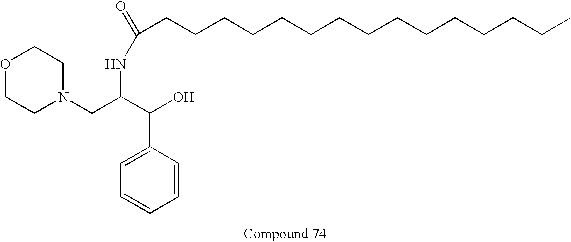 | 149022-18-4 | 19.00 | |
| 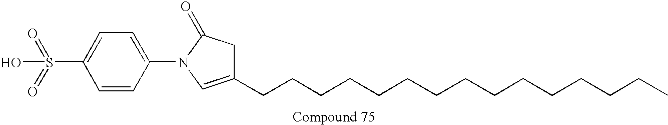 | | 1.60 | |
| 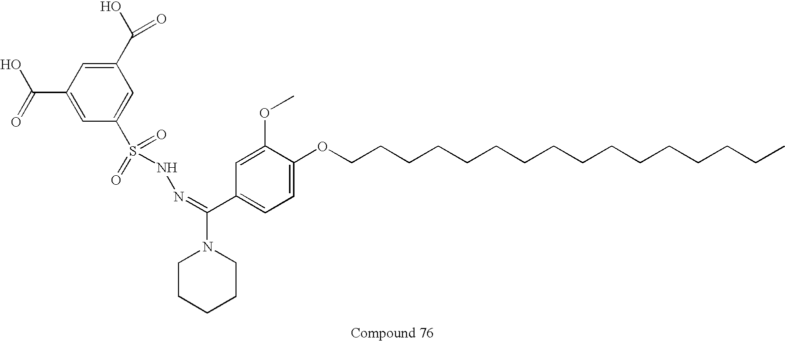 | 198066-98-7 | 3.00 | |
| 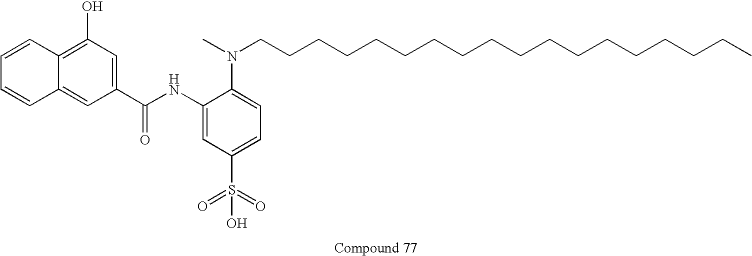 | | 0.34 | |
| 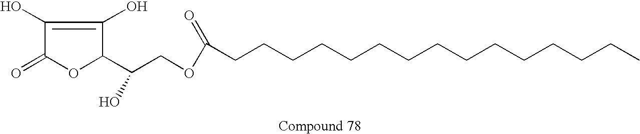 | 137-66-6 | 9.00 | |
| 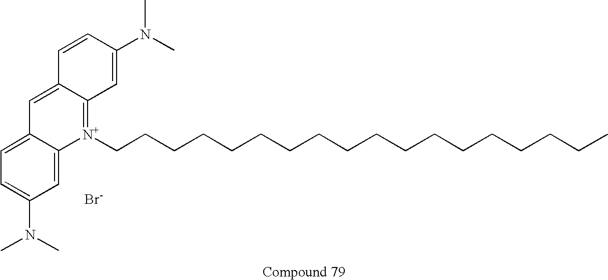 | 75168-16-0 | 1.60 | |
| 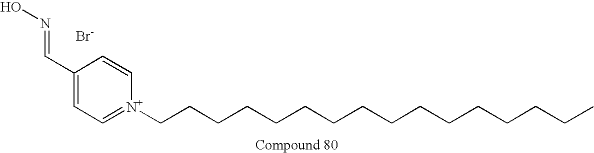 | 400837-13-0 | 17.00 | |
| 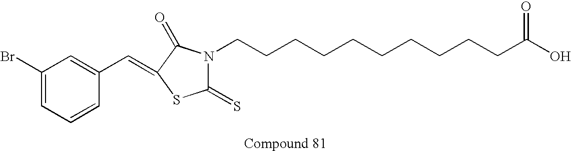 | 300377-77-9 | 18.00 | |
| 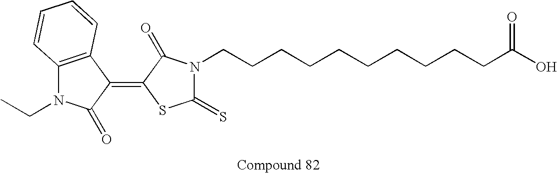 | 374094-67-4 | 17.00 | |
| 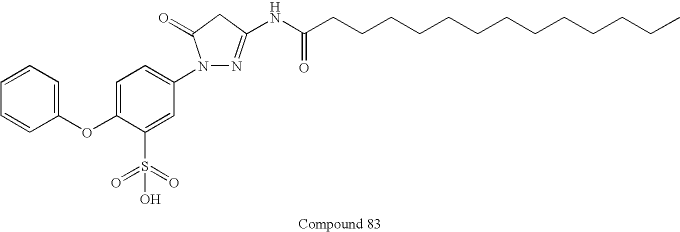 | | 0.40 | |
| 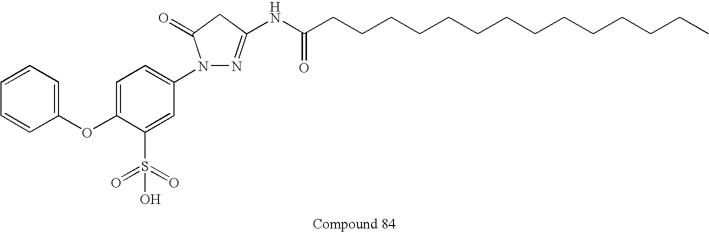 | | 0.73 | |
| 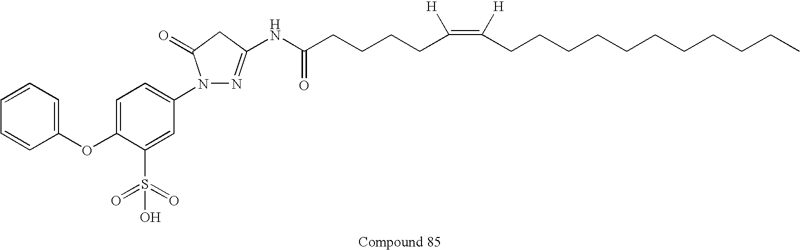 | | 0.40 | |
| 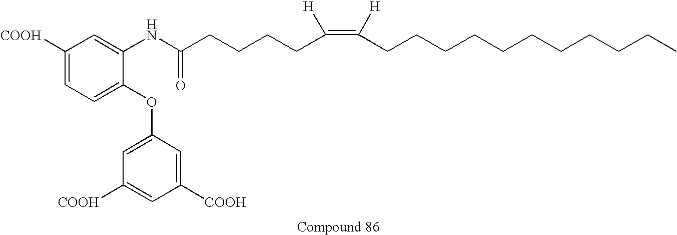 | | 2.00 | |
| 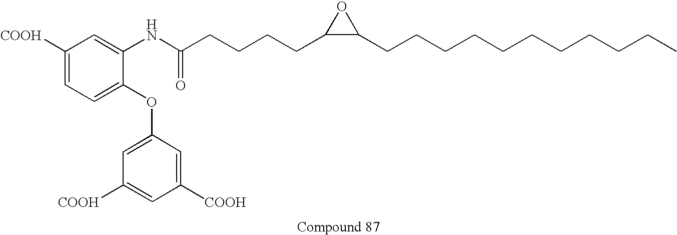 | | 0.40 | |
| 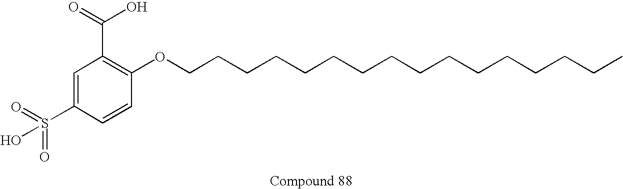 | 198971-81-2 | 4.40 | |
| 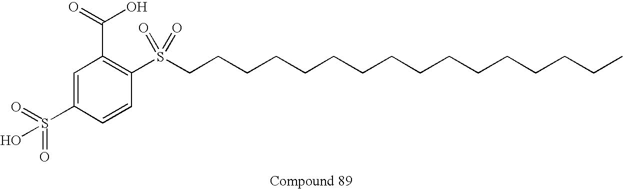 | 198971-99-2 | 5.50 | |
| 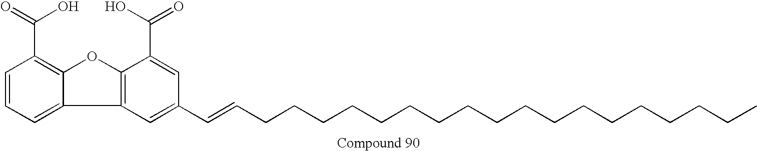 | | 8.50 | |
| 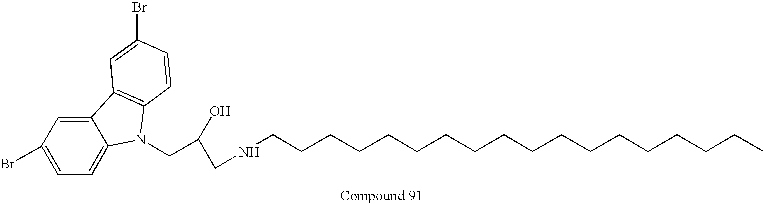 | | 15.00 | |
| 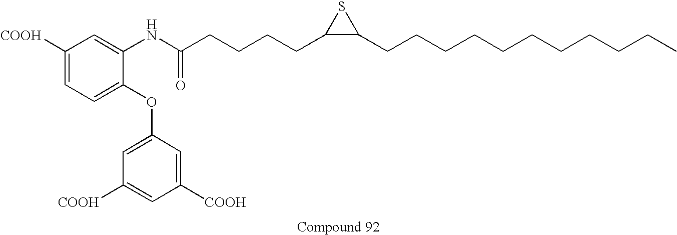 | | 0.50 | |
| 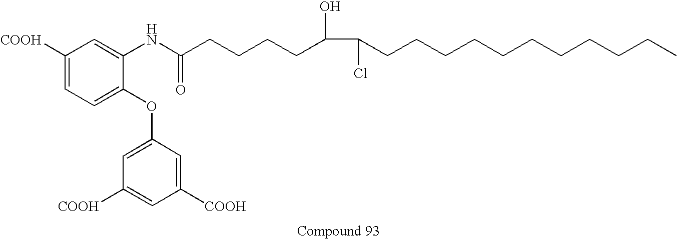 | | 9.15 | |
| 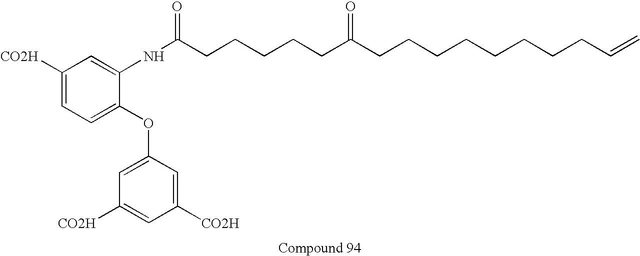 | | 18.00 | |
| 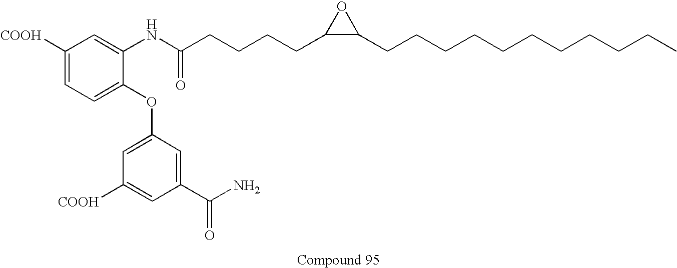 | | 1.00 | |
| 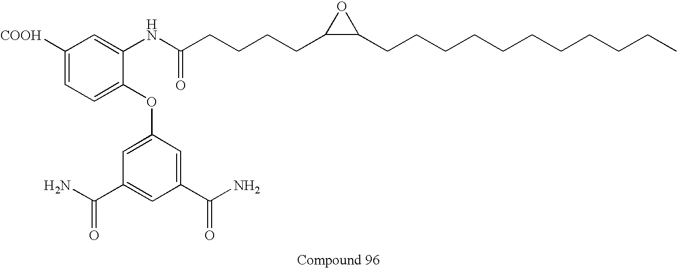 | | 1.60 | |
| 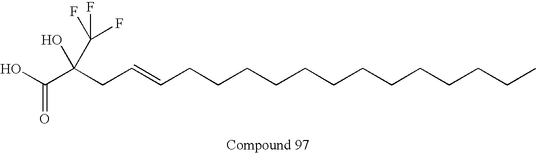 | 126882-71-1 | 6.00 | |
| 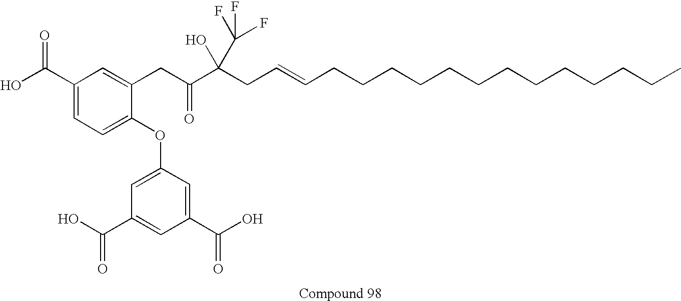 | | 1.50 | |
| 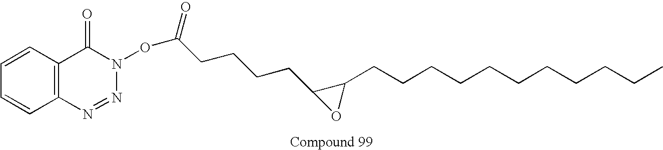 | | 16.00 | |
| 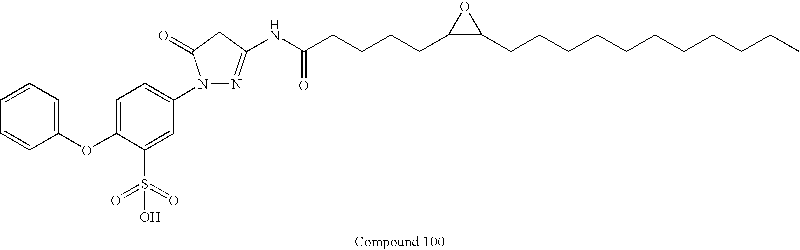 | | 0.27 | |
| 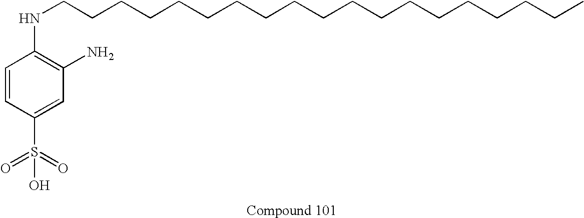 | | 2.00 | |
| 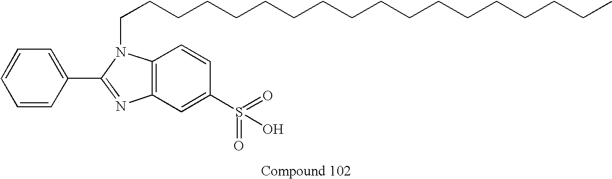 | | 0.63 | |
| 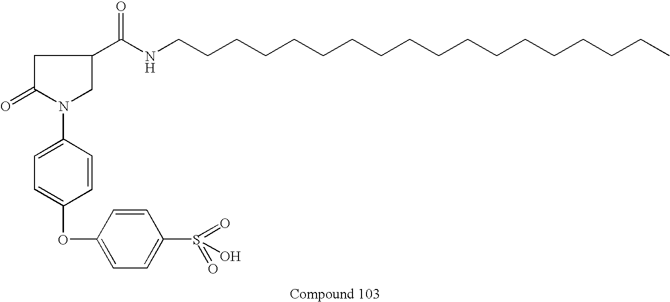 | | 7.70 | |
| 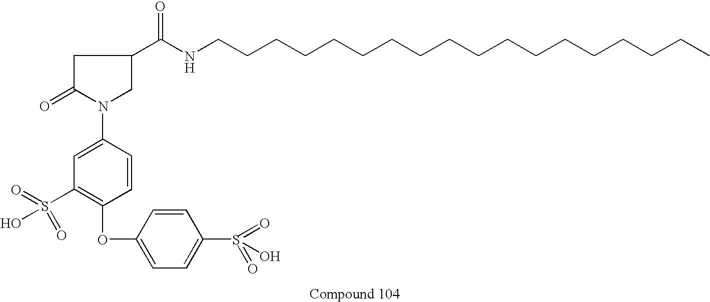 | | 0.47 | |
| 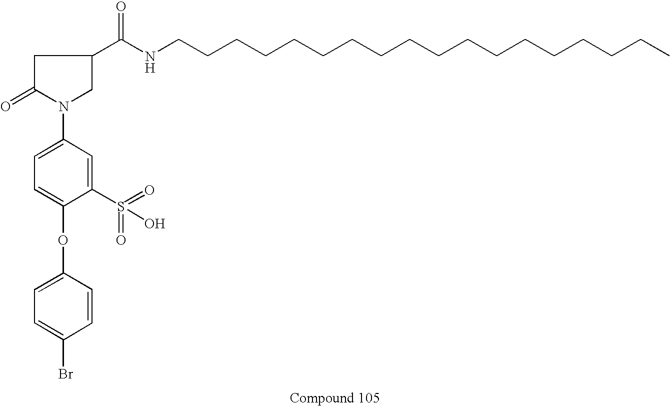 | | 0.86 | |
| 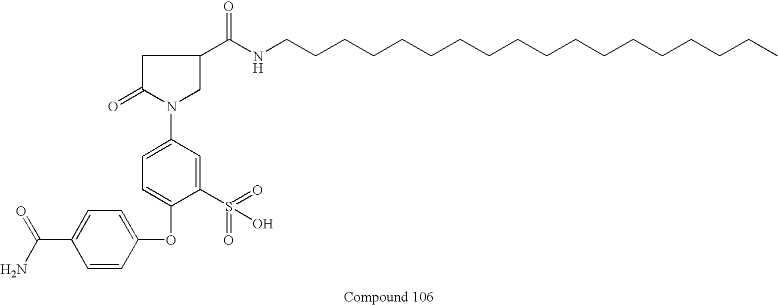 | | 0.30 | |
| 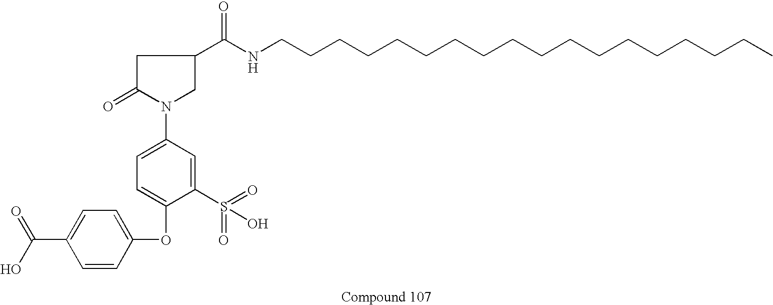 | | 0.33 | |

1. A method for treatment of a disease or disorder caused by or associated with heparanase catalytic activity, said method comprising administering to a patient in need an effective amount of a heparanase inhibitor of the general formula I, II III or IV: 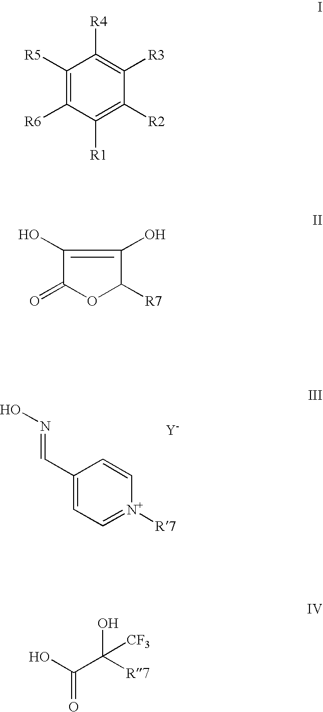
wherein
R1 is selected from the group consisting of: 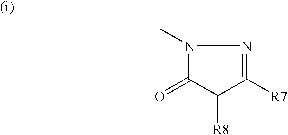
or the tautomer 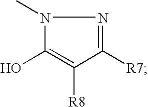
(ii) —N(R9)—CO(R10);
(iii) —CO—N(R9)(R10);
(iv) —SO₂R11; 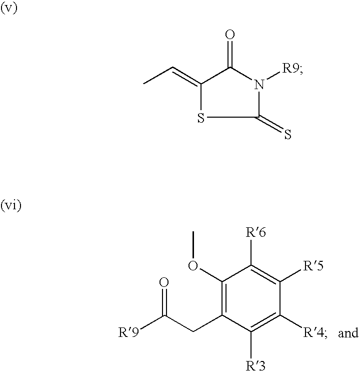
(vii) —CH(OH)—CH(NH—CO—R′7)-CH₂NR9R′9 R2, R3, R4, R5, R6, R′3, R′4, R′5 and R′6 each independently represents hydrogen, halogen, nitro, (C1-C32) alkyl, (C2-C32) alkenyl, (C6-C14) aryl, heteroaryl, —OR9′, —SR9′, —NR9R′9, —(CH₂)ₙ—NR9-COR′9, —COR′9, —COOR′9, —(CH₂)ₙ—CO—N(R9)(R′9); —SO₃R′9, —SO₂R′9, or —NHSO₂R′9;
or R1 and R2 together are a moiety selected from the group consisting of: 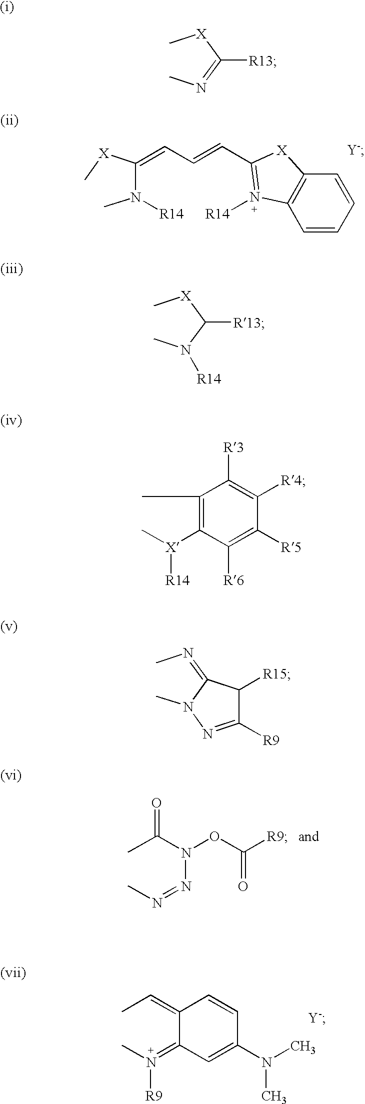
wherein X is O, S, N(R12) or C(R′12, R″12) and X′ is O or N; or each pair of R2+R3, R3+R4, R4+R5 or R5+R6, together with the carbon atoms to which they are attached, form a 5- or 6-membered aromatic ring;
R7 is selected from the group consisting of H, halogen, (C1-C32) alkyl, (C2-C32) alkenyl, (C6-C14) aryl, heteroaryl, —OR′9, —SR′9, —NR9R′9, —NR9-COR′9, —COR′9, —COOR′9, —CH(OH)—(CH₂)ₙ—O—CO—R9, —(CH₂)ₙ—NR9-COR′9, —(CH₂)ₙ—CO—N(R9)(R′9), —SO₃R′9, —SO₂R′9, —NHSO₂R′9, —N═N—(C6-C14) aryl, and 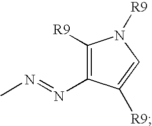
R′7 is (C1-C32) alkyl;
R″7 is (C2-C32) alkenyl;
R8 is as defined for R7;
R9 is H or (C1-C32) alkyl and R′9 is H, (C1-C32) alkyl, (C2-C32) alkenyl or (C6-C14) aryl, or R9 and R′9 as part of the radical —NR9R′9 form together with the N atom to which they are attached a 3-7 membered saturated ring, optionally further containing one or more N, S or O atoms;
R10 is selected from the group consisting of (C1-C32) alkyl, (C2-C32) alkenyl, —(CH₂)ₙ—CO—R17, —(CH₂)ₙ—NH—CO—R9-O—R′9, and 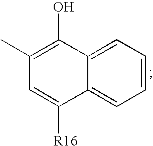
R11 is OH or 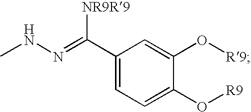
R12, R′12 and R″12 each is H or (C1-C32) alkyl, or R′12 and R″12 together are a radical 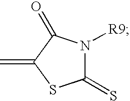
R13 is selected from the group consisting of (C1-C32) alkyl, (C6-C14) aryl, —N═CH—(C6-C14)aryl, 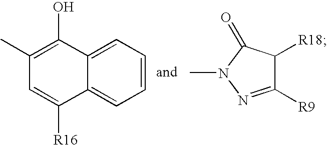
R′13 is ═O, ═NH or ═N—NH—SO₂R′9;
R14 is H, (C1-C32) alkyl, —(CH₂)ₘ—CH(OH)—CH₂—NR9R′9 or —(CH₂)ₘ—CH(OH)—(C6-C14) aryl;
R15 is H or —SO₃H;
R16 is selected from the group consisting of H, halogen, —COOH, —SO₃H, —N═N—(C6-C14) aryl, and 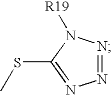
R17 is selected from the group consisting of (C1-C32) alkyl, (C6-C14) aryl, —NH—NH—CO—(C1-C32) alkyl, —NH—NH—CO—(C6-C14) aryl, —(CH₂)ₙ—NH—CO—C(R9)-O(C1-C32) alkyl, —(CH₂)ₙ—NH—CO—C(R9)-O(C6-C14) aryl, —(CH₂)ₙ—CO—(C1-C32) alkyl, and —(CH₂)ₙ—CO—(C6-C14) aryl;
R18 is H or ═N—(C6-C14) aryl;
R19 is (C6-C14) aryl;
Y⁻ is a counter ion selected from the group consisting of chloride, bromide, iodide, perchlorate, tosylate, mesylate, sulfate, phosphate and an organic anion;
n is 0 or an integer from 1 to 10; m is an integer from 1 to 10; and
any “(C1-C32) alkyl” or “(C2-C32) alkenyl” may be straight or branched and may be interrupted by one or more heteroatoms selected from O, S and/or N, and/or substituted by one or more radicals selected from the group consisting of halogen, (C3-C7) cycloalkyl, (C6-C14) aryl, nitro, OR′9, SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9;
“heteroaryl” means a radical derived from a mono- or poly-cyclic heteroaromatic ring containing 1 to 3 heteroatoms selected from the group consisting of O, S and N; and any “aryl” or “heteroaryl” may be substituted by one or more radicals selected from the group consisting of halogen, (C6-C14) aryl, (C1-C32)alkyl, nitro, —OR′9, —SR′9, —COR′9, COOR′9, —OSO₃R′9, —SO₃R′9, —SO₂R′9, —NHSO₂R′9, —NR9R′9, —(CH₂)ₙ—NR9-COR′9, and —(CH₂)ₙ—CO—NR9R′9;
or a pharmaceutically acceptable salt thereof.

2. The method according to claim 1, comprising administering a compound of the formula Ia or I′a: 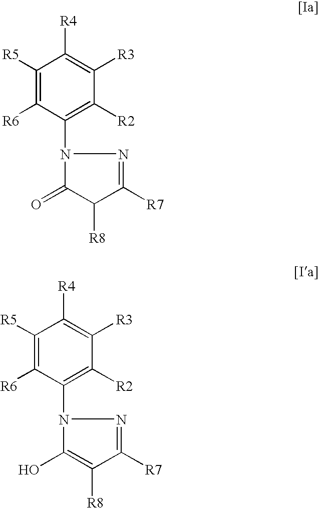
wherein
R2 is H, halogen, —NH₂ or —SO₃H;
R3 is H or —SO₃H;
R4 is H, halogen, —SO₃H, —SO₂—(C10-C22) alkyl or —O(C6-C14) aryl, wherein the aryl is unsubstituted or substituted by —O(C1-C8) alkyl;
R5 is H; R6 is H or halogen;
R7 is selected from the group consisting of:
(i) H;
(ii) (C10-C22) alkyl;
(iii) —COOH;
(iv) —NR9-COR′9, wherein R9 is H and R′9 is (C10-C22) alkyl optionally substituted by epoxy, (C10-C22) alkenyl optionally substituted by —COOH, or (C6-C14) aryl optionally substituted by —SO₃H or —NH—CO—(C10-C22) alkyl; and
(v) (C6-C14) aryl optionally substituted by —SO₃H or by —NR9-COR′9, wherein R9 is H and R′9 is (C10-C22) alkyl;
R8 is selected from the group consisting of:
(i) H;
(ii) halogen;
(iii) (C2-C6) alkyl;
(iv) —O(C10-C22) alkyl;
(v) (C6-C14) aryl optionally substituted by one or more halogen, —OR′9, —COOR′9, —SO₃R′9, —NR9R′9 or —NR9COR′9, wherein R9 and R′9 each independently is H or (C10-C22) alkyl; 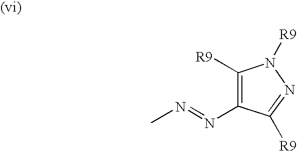
wherein R9 each independently is H or (C1-C12) alkyl; and
(vii) —N═N—(C6-C14) aryl optionally substituted by one or more halogen, —OR′9, —COOR′9, —SO₃R′9, —NHSO₂R′9, —NR9R′9, or —NR9-CO—R′9, wherein R9 and R′9 each independently is H or (C1-C6) alkyl, or R′9 is (C6-C14) aryl substituted by methyl;
wherein any “(C10-C22) alkyl” as defined in R4, R7 and R8 may be straight or branched and may be interrupted by one or more heteroatoms selected from the group consisting of O, S and N, and/or may be substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 in this context is H or (C1-C32) alkyl and R′9 is H, (C1-C32) alkyl, (C2-C32) alkenyl or (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

3. The method according to claim 2, wherein:
R2 is H, Cl, —NH₂, or —SO₃H;
R3 is H or —SO₃H;
R4 is H, Cl, —SO₃H, —SO₂C₁₆H₃₃ or phenoxy optionally substituted by ethoxy;
R5 is H, —COOH or —SO₃H;
R6 is H or Cl;
R7 is selected from the group consisting of:
(i) H;
(ii) (C17-C20) alkyl;
(iii) —COOH;
(iv) —NR9-COR′9, wherein R9 is H and R′9 is (C11-C20) alkyl optionally substituted by epoxy, (C16-C20) alkenyl optionally substituted by —COOH, or phenyl optionally substituted by —SO₃H or —NH—CO—C₁₇H₃₅;
(v) phenyl, optionally substituted by —SO₃H or by —NR9-COR′9, wherein R9 is H and R′9 is (C17-C20) alkyl; and
R8 is selected from the group consisting of:
(i) H;
(ii) Br;
(iii) isopropyl;
(iv) —OC₁₆H₃₃;
(v) phenyl, optionally substituted by one or more halogen, —OR′9, —COOR′9, —SO₃R′9, —NR9R′9 or —NR9COR′9, wherein R9 and R′9 each independently is H or —C₁₆H₃₃; 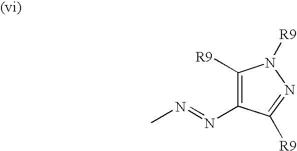
wherein R9 each independently is H, methyl or decenyl; and
(vii) —N═N-phenyl optionally substituted by one or more Cl, —OR′9, —COOR′9, —SO₃R′9, —NHSO₂R′, —NR9R′9, or —NR9-CO—R′9, wherein R9 and R′9 each independently is H, methyl or ethyl, or R′9 is phenyl substituted by methyl.

4. The method according to claim 3, wherein said compound of formula Ia is selected from the group of compounds herein designated Compounds Nos. 1, 5-22, 24-30, 54, 56, 69, 71, 83, 84, 85 and 100, or said compound of the formula I′a is the herein designated Compound No. 32.

5. (canceled)

6. The method according to claim 1, comprising administering a compound of the formula Ib: 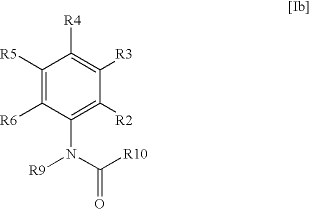
wherein
R2 is selected from the group consisting of:
(i) H;
(ii) halogen;
(iii) —OH;
(iv) —O(C10-C22) alkyl;
(v) —COOH;
(vi) —NR9R′9, wherein R9 and R′9 each independently is H, or R9 is (C1-C6) alkyl and R′9 is H or (C10-C22) alkyl; and
(vii) —O(C6-C14) aryl optionally substituted by one or more —COOH or —CO—NH₂;
R3 is H or —COOH;
R4 is selected from the group consisting of:
(i) H;
(ii) —SO₃H
(iii) —O(C6-C14) aryl optionally substituted by one or more COOH;
(iv) —S(C6-C14) aryl optionally substituted by one or more COOH; and
(v) —NR9-CO—R′9, wherein R9 and R′9 each independently is H or (C10-C22) alkyl;
R5 is H, —COOH, —SO₃H, or —NHSO₂—(C6-C14) aryl optionally substituted by one or more —COOH;
R6 is H;
R9 is H or (C10-C22) alkyl;
R10 is selected from the group consisting of:
(i) (C10-C22) alkyl optionally substituted by one or more radicals selected from the group consisting of halogen, OH, epoxy and epithio; 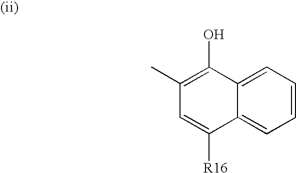
wherein R16 is H, halogen, —COOH, —SO₃H, —S-tetrazol-5-yl optionally substituted by phenyl, or —N═N—(C6-C14) aryl optionally substituted by one or more radicals selected from the group consisting of halogen, (C1-C6) alkyl, (C6-C14) aryl, —OH, —COOH, —COOR′9, —OR′9 and —NHSO₂R′9, wherein R′9 is (C1-C6) alkyl or phenyl optionally substituted by (C1-C6) alkyl;
(iii) —CH₂—CO—R17, wherein R17 is (C10-C22) alkyl, (C6-C14) aryl optionally substituted by —O—(C10-C22) alkyl or by —NH—CO—(C10-C22) alkyl; or —NH—NH—CO—(C10-C22) alkyl;
(iv) —NH—(C10-C22) alkyl; and
(v) (C10-C22) alkenyl optionally substituted by oxo;
wherein any “(C10-C22) alkyl” as defined in R2, R4, R9 and R10 may be straight or branched and may be interrupted by one or more heteroatoms selected from the group consisting of O, S and N, and/or may be substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, —(C2-C32) alkenyl and —(C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

7. The method according to claim 6, wherein:
R2 is selected from the group consisting of:
(i) H;
(ii) Cl;
(iii) —OH;
(iv) —OC₁₈H₃₇;
(v) —COOH;
(vi) —NR9R′9, wherein R9 is H or methyl and R′9 is —C₁₈H₃₇; and
(vii) phenoxy optionally substituted by one or more —COOH or —CO—NH₂;
R3 is H or —COOH;
R4 is selected from the group consisting of:
(i) H;
(ii) —SO₃H
(iii) phenoxy optionally substituted by one or more —COOH;
(iv) phenylthio optionally substituted by one or more —COOH; and
(v) —NR9-CO—R′9, wherein R9 and R′9 each independently is H or —C₁₇H₃₅;
R5 is H, —COOH, —SO₃H, —NHSO₂-phenyl optionally substituted by one or more —COOH;
R6 is H;
R9 is H or —C₁₈H₃₇;
R10 is selected from the group consisting of:
(i) —C₁₇H₃₅, optionally substituted by one or more radicals selected from the group consisting of Cl, —OH, epoxy and epithio; 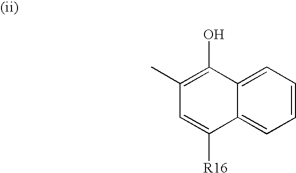
wherein R16 is H, Br, —COOH, —SO₃H, —S-tetrazol-5-yl optionally substituted by phenyl, or —N═N-phenyl optionally substituted by one or more radicals selected from the group consisting of Cl, methyl, phenyl, —OH, —COOH, —COOR′9, —OR′9 and —NHSO₂R′9, wherein R′9 is methyl or phenyl optionally substituted by methyl;
(iii) —CH₂—CO—R17, wherein R17 is selected from the group consisting of —C₁₇H₃₅, —C₁₈H₃₅, phenyl optionally substituted by —OC₁₈H₃₇ or by —NH—CO—(C15-C20) alkyl, preferably —NH—CO—C₁₇H₃₅, and —NH—NH—CO—(C15-C20) alkyl, preferably —NH—NH—CO—C₁₇H₃₅;
(iv) —NH—C₁₈H₃₇; and
(v) (C16-C20) alkenyl, preferably —C₁₇H₃₃ or —C₁₆H₃₁, optionally substituted by oxo.

8. The method according to claim 7, comprising administration of: (i) a compound wherein R10 is —C₁₇H₃₅, selected from the group of compounds herein designated Compounds Nos. 61, 87, 92, 93, 95 and 96; (ii) a compound wherein R10 is 1-hydroxy-4-R18-2-naphthyl, selected from the group of compounds herein designated Compounds Nos. 3, 33, 34, 40, 41, 43, 45, 46, 47, 49, 50, 52, 53, 55, 62, 63 and 77; (iii) a compound wherein R10 is —CH₂—CO—R17, selected from the group of compounds herein designated Compounds Nos. 2, 23, 44, 51, 60 and 64; (iv) the compound herein designated Compound No. 70, wherein R10 is —NH—C₁₈H₃₇; or (v) a compound wherein R10 is (C10-C22) alkenyl, selected from the group of compounds herein designated Compounds Nos. 86 and 94.

9-12. (canceled)

13. The method according to claim 1, comprising administration of a compound of the formula Ic: 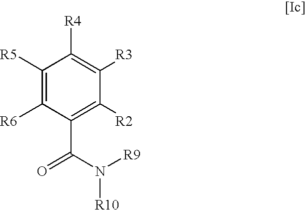
wherein
R2, R3, R4, R5, and R6 each independently represents hydrogen, halogen, nitro, (C1-C32) alkyl, (C2-C32) alkenyl, (C6-C14) aryl, heteroaryl, —OR9′, —SR9′, —NR9R′9, —(CH₂)ₙ—NR9-COR′9, —COR′9, —COOR′9, —(CH₂)ₙ—CO—N(R9)(R′9); —SO₃R′9, —SO₂R′9, or —NHSO₂R′9;
or R3 and R4 together with the carbon atoms to which they are attached form a condensed benzene ring;
R9 is H or (C1-C32) alkyl and R′9 is H, (C1-C32) alkyl, (C2-C32) alkenyl or (C6-C14) aryl, or R9 and R′9 as part of the radical —NR9R′9 form together with the N atom to which they are attached a 3-7 membered saturated ring, optionally further containing one or more N, S or O atoms;
R10 is
(i) (C10-C22) alkyl; or
(ii) —(CH₂)ₙ—NH—CO—R9-O—O′9, wherein R9 is (C1-C6) alkyl, R′9 is (C6-C14) aryl substituted by —C₁₅H₃₁; and n is an integer of 1 to 6;
and wherein the “(C1-C32) alkyl” and “(C2-C32) alkenyl” as defined in R2 to R6 and R9 and the “(C10-C22) alkyl” as defined in R10 may be straight or branched and may be interrupted by one or more heteroatoms selected from the group consisting of O, S and N, and/or may be substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10; and wherein any “(C6-C14) aryl” as defined in R2 to R6 and R9 may be substituted by one or more radicals selected from the group consisting of halogen, (C6-C14) aryl, (C1-C32) alkyl, nitro, OR′9, SR′9, —COR′9, COOR′9, —SO₃R′9, —SO₂R′9, —NHSO₂R′9, —NR9R′9, —(CH₂)ₙ—NR9-COR′9, and —(CH₂)ₙ—CO—NR9R′9.

14. The method according to claim 13, wherein
R2 is OH;
R3 and R4 together with the carbon atoms to which they are attached form a condensed benzene ring;
R5 is H or —SO₃H;
R6 and R9 each is H; and
R10 is
(i) —C₁₈H₃₇; or
(ii) —(CH₂)ₙ—NH—CO—R9-O—R′9, wherein R9 is —CH(C₂H₅) and R′9 is phenyl substituted by —C₁₅H₃₁; and n is 3.

15. The method according to claim 14, comprising administering the compound herein designated Compound No. 31 or No. 72.

16. The method according to claim 1, comprising administering a compound of the formula Id: 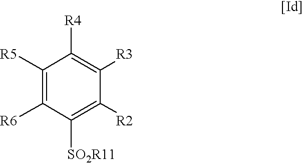
wherein R2 is H;
R3 is H, —COOH, —NH₂, or 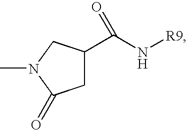
wherein R9 is (C10-C22) alkyl;
R4 is selected from the group consisting of:
(i) H;
(ii) —O—(C10-C22) alkyl;
(iii) —NH—(C10-C22) alkyl;
(iv) —SO₂—(C10-C22) alkyl; 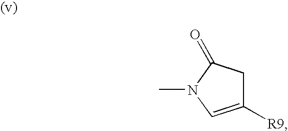
wherein R9 is (C10-C22) alkyl; and
(v) phenoxy optionally substituted by —SO₃H or 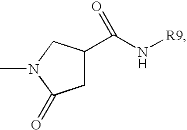
or both;
wherein R9 is (C10-C22) alkyl;
R5 is H, —COOH or —NH₂;
R6 is H or phenoxy optionally substituted by halogen, —COOH or —CONH₂;
R11 is OH or 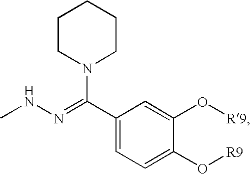
wherein R9 is (C10-C22) alkyl and R′9 is (C1-C6) alkyl;
wherein any “(C10-C22) alkyl” as defined in R4 and R9 may be straight or branched and may be interrupted by one or more heteroatoms selected from the group consisting of O, S and N, and/or may be substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

17. The method according to claim 16, wherein:
R2 is H;
R3 is H, —COOH, —NH₂ or 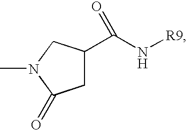
wherein R9 is —C₁₈H₃₇;
R4 is selected from the group consisting of:
(i) H;
(ii) —O—C₁₆H₃₃;
(iii) —NH—C₁₉H₃₉;
(iv) —SO₂—C₁₆H₃₃; 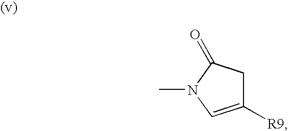
wherein R9 is —C₁₅H₃₁; and
(vi) phenoxy, optionally substituted by —SO₃H or 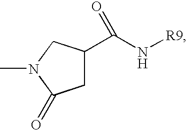
or both;
wherein R9 is —C₁₈H₃₇;
R5 is H, —COOH, or —NH₂;
R6 is H or phenoxy optionally substituted by halogen, —COOH or —CONH₂; and
R11 is OH or 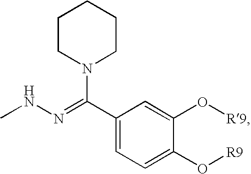
wherein R9 is —C₁₆H₃₃ and R′9 is methyl.

18. The method according to claim 17, comprising administering a compound selected from the group of compounds herein designated Compounds Nos. 75, 76, 88, 89, 101, 103, 104, 105, 106 and 107.

19. The method according to claim 1, comprising administering a compound of the formula Ie: 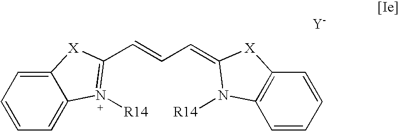
wherein
X is O or S;
R14 is (C10-C22) alkyl; and
Y⁻ is a counter ion selected from the group consisting of chloride, bromide, iodide, perchlorate, tosylate, mesylate, sulfate, phosphate and an organic anion;
and wherein the “(C10-C22) alkyl” as defined in R14 may be straight or branched and may be interrupted by one or more heteroatoms selected from the group consisting of O, S and N, and/or may be substituted by one or more radicals selected from the group consisting of halogen, (C3-C7) cycloalkyl, preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

20. The method according to claim 19 comprising administering a compound of formula Ie, wherein X is O or S; R14 is —C₁₈H₃₇; and Y⁻ is perchlorate, said compounds herein designated as Compound No. 66 or 67, respectively.

21. (canceled)

22. The method according to claim 1, comprising administering a compound of the formula If: 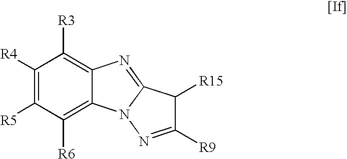
wherein
R3 and R5 each is H;
R4 is H, —COOH or —SO₃H;
R6 is H or —COOH;
R9 is H or (C10-C22) alkyl; and
R15 is H or —SO₃H;
and wherein the “(C10-C22) alkyl” as defined in R9 may be straight or branched and may be interrupted by one or more heteroatoms selected from the group consisting of O, S and N, and/or may be substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

23. The method according to claim 22, wherein R3 and R5 are H; R6 is H or —COOH; R4 is H, COOH or —SO₃H; R9 is H or —C₁₇H₃₅; and R15 is H or —SO₃H.

24. The method according to claim 23, comprising administering a compound selected from the compounds herein designated Compounds Nos. 4, 35 and 36.

25. The method according to claim 1, comprising administering a compound of the formula Ig: 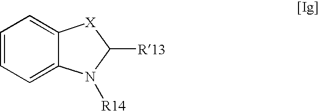
wherein
X is NR12 or CR′12R″12;
R12 is (C10-C22) alkyl;
R′12 and R″12 each is (C1-C6) alkyl, or R′12 and R″12 together are a radical 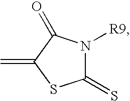
wherein R9 is H or (C10-C22) alkyl substituted by —COOH;
R′13 is selected from the group consisting of ═O, ═NH and ═N—NH—SO₂—(C6-C14) aryl, wherein the aryl is either substituted by —COOH and —O—(C10-C22) alkyl, or by —NH—SO₂-phenyl, wherein the phenyl is substituted by —COOH and —O—(C10-C22) alkyl; and
R14 is (C1-C8) alkyl or —CH₂—CH(OH)—(C6-C14) aryl substituted by one or more (C1-C6) alkoxy;
wherein any “(C10-C22) alkyl” as defined in R12 and R′13 may be straight or branched and may be interrupted by one or more heteroatoms selected from the group consisting of O, S and N, and/or may be substituted by one or more radicals selected from the group consisting of halogen, (C3-C7) cycloalkyl, preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

26. The method according to claim 25, wherein
X is NR12 or CR′12R″12;
R12 is —C₁₆H₃₃;
R′12 and R″12 each is methyl, or R′12 and R″12 together are a radical 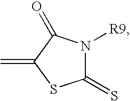
wherein R9 is H or —C₁₀H₂₀—COOH;
R′13 is ═O, ═NH or ═N—NH—SO₂-phenyl, wherein the phenyl is either substituted by —COOH and —OC₁₈H₃₇, or by —NH—SO₂-phenyl, wherein the phenyl is substituted by —COOH and —OC₁₈H₃₇; and
R14 is methyl or ethyl, or —CH₂—CH(OH)-phenyl substituted by one or more methoxy groups.

27. The method according to claim 26, comprising administering a compound selected from the group of compounds herein designated Compounds Nos. 48, 59 65 and 82.

28. The method according to claim 1, comprising administering a compound of the formula Ih: 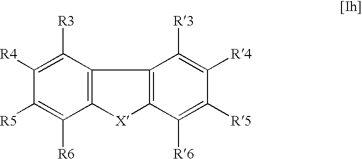
wherein
X′ is O or NR14;
R3, R4, R5, R′3 and R′5 each is H or halogen;
R′4 is H, halogen or (C10-C22) alkenyl;
R6 and R′6 each is H or —COOH; and
R14 is (C10-C22) alkyl interrupted by one or more N atoms and substituted by hydroxy;
and wherein the “(C10-C22) alkenyl” as defined in R′4 may be straight or branched and may be interrupted by one or more heteroatoms selected from the group consisting of O, S and N, and/or may be substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, —COOR′9, —OSO3R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

29. The method according to claim 28, wherein:
X′ is O or NR14;
R3, R4, R5, R′3 and R′5 each is H, Cl or Br;
R′4 is selected from the group consisting of H, Cl, Br and —C₂₀H₃₉;
R6 and R′6 each is —H or —COOH; and
R14 is C₁₀H₂₁—NH—CH₂—CH(OH)—CH₂— or C₁₈H₃₇—NH—CH₂—CH(OH)—CH₂—.

30. The method according to claim 29 comprising administering a compound selected from the group of compounds herein designated Compounds Nos. 68, 90 and 91.

31. The method according to claim 1, comprising administering a compound of the formula Ii: 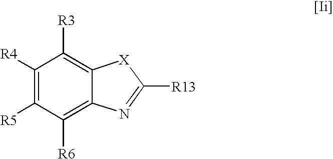
wherein
X is O, S or NR12;
R4 is H or —SO₃H;
R6 is H;
R3 is H or —COOH;
R5 is H, —COOH or —SO₃H;
R12 is H or (C10-C22) alkyl;
R13 is selected from the group consisting of:
(i) (C1-C6) alkyl; 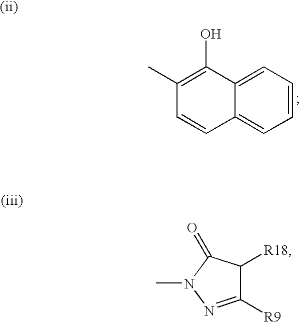
wherein R9 is (C10-C22) alkyl and R18 is H or ═N—(C6-C14) aryl wherein the aryl is optionally substituted by —NR9R′9, wherein R9 and R′9 each is (C1-C6) alkyl;
(iv) (C6-C14) aryl, optionally substituted by, 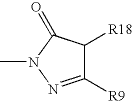
wherein R9 is (C10-C22) alkyl and R18 is ═N—(C6-C14) aryl, wherein the aryl is optionally substituted by —NR9R′9, wherein R9 and R′9 each is (C1-C6) alkyl; and
(v) —N═CH—(C6-C10) aryl substituted by one or more halogen and —OH or by one or more —OH and nitro;
wherein any “(C10-C22) alkyl” as defined in R12 and R13 may be straight or branched and may be interrupted by one or more heteroatoms selected from the group consisting of O, S and N, and/or may be substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

32. The method according to claim 31, wherein:
X is O, S or NR12;
R4 is H or —SO₃H;
R6 is H;
R3 is H or —COOH;
R5 is H, —COOH or —SO₃H;
R12 is H, —C₁₆H₃₃ or —C₁₈H₃₇;
R13 is selected from the group consisting of:
(i) methyl; 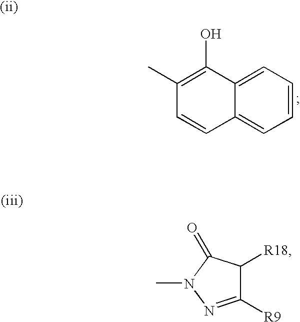
wherein R9 is —C₁₇H₃₅ and R18 is H or ═N-phenyl, wherein the phenyl is optionally substituted by —NR9R′9, wherein R9 and R′9 each is ethyl;
(iv) phenyl optionally substituted by 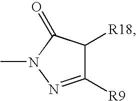
wherein R9 is —C₁₇H₃₅ and R18 is ═N-phenyl, wherein the phenyl is optionally substituted by —NR9R′9, wherein R9 and R′9 each is ethyl; and
(v) —N═CH-phenyl optionally substituted by —OH and one or more Cl or Br, or naphthyl optionally substituted by —OH or nitro, or both.

33. The method according to claim 32, comprising administering a compound selected from the compounds herein designated Compounds Nos. 37, 38, 39, 42, 57, 58, 73 and 102.

34. The method according to claim 1, comprising administering a compound of the formula Ij: 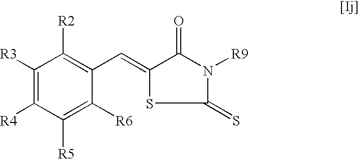
wherein
R2, R4, R5 and R6 each is H;
R3 is H or halogen; and
R9 is H or (C10-C22) alkyl substituted by —COOH.

35. The method according to claim 34, wherein R2, R4, R5 and R6 each is H; R3 is H or Br; and R9 is H or —C₁₀H₂₀—COOH.

36. The method according to claim 35, comprising the compound herein designated Compound No. 81.

37. The method according to claim 1, comprising administering a compound of the formula Ik: 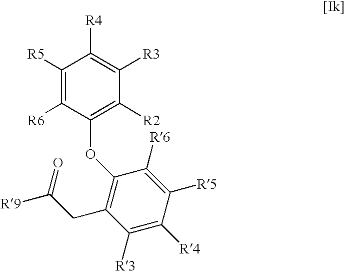
wherein
R2, R4, R6, R′3, R′5 and R′6 each is H;
R3, R5 and R′4 each is H or —COOH; and
R′9 is (C10-C22) alkenyl optionally substituted by OH and —CF₃; and wherein the “(C10-C22) alkenyl” as defined in R′9 may be straight or branched and may be interrupted by one or more heteroatoms selected from the group consisting of O, S and N, and/or may be substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

38. The method according to claim 37, wherein R2, R4, R6, R′3, R′5 and R′6 each is H; R3, R5 and R′4 each is —COOH; and R′9 is C₁₇H₃₁ optionally substituted by OH and —CF₃.

39. The method according to claim 38, comprising administering the compound herein designated Compound No. 98.

40. The method according to claim 1, comprising administering a compound of the formula Il: 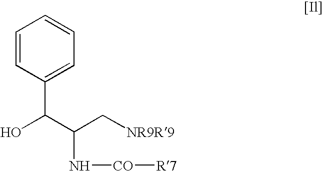
wherein
R′7 is (C10-C22) alkyl; and
R9 and R′9 together with the N atom to which they are attached form a 3-7 membered saturated ring, optionally containing a further O, N or S atom;
and wherein any “(C10-C22) alkyl” as defined in R′7, may be straight or branched and may be interrupted by one or more heteroatoms selected from the group consisting of O, S and N, and/or may be substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

41. The method according to claim 40, wherein R′7 is (C10-C22) alkyl and R9 and R′9 together with the N atom to which they are attached form a morpholine ring.

42. The method according to claim 41, comprising administering the compound herein designated Compound No. 74.

43. The method according to claim 1, comprising administering a compound of the formula Im: 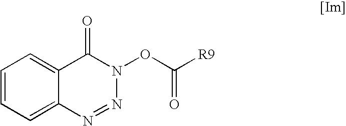
wherein
R9 is (C10-C22) alkyl, or (C10-C22) alkyl interrupted by one or more heteroatoms selected from the group consisting of O, S and N, or (C10-C22) alkyl substituted or both interrupted and substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

44. The method according to claim 43, wherein R9 is —C₁₇H₃₃ optionally substituted by epoxy.

45. The method according to claim 44, comprising administering the compound herein designated Compound No. 99.

46. The method according to claim 1, comprising administering a compound of the formula In: 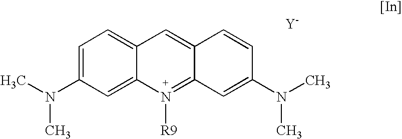
wherein
R9 is (C10-C22) alkyl; and
Y⁻ is a counter ion selected from the group consisting of chloride, bromide, iodide, perchlorate, tosylate, mesylate, sulfate, phosphate and an organic anion;
and wherein the “(C10-C22) alkyl” as defined in R9 may be straight or branched and may be interrupted by one or more heteroatoms selected from the group consisting of O, S and N, and/or may be substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or —(C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

47. The method according to claim 46, comprising administering the compound herein designated Compound No. 79, wherein R9 is —C₁₈H₃₇ and Y⁻ is bromide.

48. The method according to claim 1, comprising administering a compound of the general formula II: 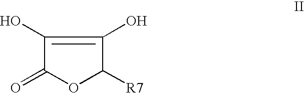
wherein
R7 is —CH(OH)—CH₂—O—CO—R9 and R9 is (C10-C22) alkyl;
and wherein the “(C10-C22) alkyl” as defined in R9 may be straight or branched and may be interrupted by one or more heteroatoms selected from the group consisting of O, S and N, and/or may be substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, —COOR′9, —OSO3R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

49. The method according to claim 48, comprising administering the compound herein designated Compound No. 78, wherein R7 is —CH(OH)—CH₂—O—CO—R9 and R9 is —C₁₅H₃₁.

50. The method according to claim 1, comprising administering a compound of the general formula III: 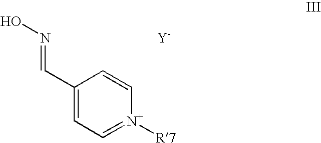
wherein
R′7 is (C10-C22) alkyl; and
Y⁻ is a counter ion selected from the group consisting chloride, bromide, iodide, perchlorate, tosylate, mesylate, sulfate, phosphate and an organic anion;
and wherein the “(C10-C22) alkyl” as defined in R′7 may be straight or branched and may be interrupted by one or more heteroatoms selected from the group consisting of O, S and N, and/or may be substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

51. The method according to claim 50, comprising administering the compound herein designated Compound No. 80, wherein R′7 is —C₁₆H₃₃, and Y⁻ is bromide.

52. The method according to claim 1, comprising administering a compound of the general formula IV: 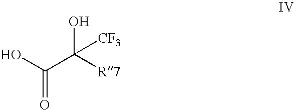
wherein R″7 is (C2-C32) alkenyl, that may be straight or branched and may be interrupted by one or more heteroatoms selected from the group consisting of O, S and N, and/or may be substituted by one or more radicals selected from the group consisting of halogen, (C3-C7) cycloalkyl preferably cyclopropyl, (C6-C14) aryl, nitro, —OR′9, —SR′9, epoxy, epithio, oxo, —COR′9, —COOR′9, —OSO₃R′9, —SO₃R′9, —SO₂R′9, —NHSO₂R′9, —NR9R′9, aziridine, ═N—OR′9, ═N—NR9R′9, —NR9-NR9R′9, —(CH₂)ₙ—NR9-COR′9, —(CH₂)ₙ—CO—NR9R′9, —OPO₃R9R′9, —PO₂HR′9 and —PO₃R9R′9; and wherein R9 is H or (C1-C32) alkyl and R′9 is selected from the group consisting of H, (C1-C32) alkyl, (C2-C32) alkenyl and (C6-C14) aryl, or R9 and R′9 as part of the radical —NR9R′9 form together with the N atom to which they are attached a 3-7 membered saturated ring, optionally further containing one or more N, S or O atoms; and n is 0 or an integer from 1 to 10.

53. The method according to claim 52, comprising administering the compound herein designated Compound No. 97, wherein R″7 is —C₁₆H₃₁.

54. The method according to claim 1, for inhibition of angiogenesis.

55. The method according to claim 1, for treatment or inhibition of a malignant cell proliferative disease or disorder.

56. The method according to claim 55, for the treatment or inhibition of non-solid cancer.

57. The method according to claim 55, for the treatment or inhibition of a solid tumor.

58. The method according to claim 56, for treating or inhibiting tumor formation, primary tumors, tumor progression or tumor metastasis.

59. The method according to claim 1, for treatment of ophthalmologic disorders selected from the group consisting of diabetic retinopathy and macular degeneration, particularly age-related macular degeneration.

60. The method according to claim 1, for inhibiting or treating a cell proliferative disease or disorder.

61. The method according to claim 1, for inhibiting or treatment of a disease or disorder selected from the group consisting of polyps, multiple exostosis, hereditary exostosis, retrolental fibroplasia, hemangioma, reperfusion of gastric ulcer and arteriovenous malformation.

62. The method according to claim 1, for contraception or for inducing abortion at early stages of pregnancy.

63. The method according to claim 1, for treatment of, or amelioration of, inflammatory symptoms in any disease, condition or disorder where immune and/or inflammation suppression is beneficial.

64. The method according to claim 63, for treatment of, or amelioration of, inflammatory symptoms in the joints, musculoskeletal or connective tissue disorders.

65. The method according to claim 63, for treatment of, or amelioration of, inflammatory symptoms associated with hypersensitivity, allergic reactions, asthma, atherosclerosis, otitis or other otorhinolaryngological diseases, dermatitis or other skin diseases, posterior and anterior uveitis, conjunctivitis, optic neuritis, scleritis or other immune and/or inflammatory ophthalmic diseases.

66. The method according to claim 1, for treatment of, or amelioration of, an autoimmune disease.

67. The method according to claim 66, wherein said autoimmune disease is Eaton-Lambert syndrome, Goodpasture's syndrome, Grave's disease, Guillain-Barré syndrome, autoimmune hemolytic anemia (AIHA), hepatitis, insulin-dependent diabetes mellitus (IDDM), systemic lupus erythematosus (SLE), multiple sclerosis (MS), myasthenia gravis, plexus disorders e.g. acute brachial neuritis, polyglandular deficiency syndrome, primary biliary cirrhosis, rheumatoid arthritis, scleroderma, thrombocytopenia, thyroiditis e.g. Hashimoto's disease, Sjögren's syndrome, allergic purpura, psoriasis, mixed connective tissue disease, polymyositis, dermatomyositis, vasculitis, polyarteritis nodosa, polymyalgia rheumatica, Wegener's granulomatosis, Reiter's syndrome, Behçet's syndrome, ankylosing spondylitis, pemphigus, bullous pemphigoid, dermatitis herpetiformis, Crohn's disease or autism.

68-135. (canceled)

136. A compound selected from the group of compounds herein designated Compounds Nos. 12, 18, 27, 37, 48, 50, 61-63, 70, 71, 75, 77, 83-87, 90-96 and 98-107.

137. The method according to claim 56, wherein said non-solid cancer is a hematopoietic malignancy selected from the group consisting of acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma.

138. The method according to claim 57, wherein said solid tumor is a tumor in lip or oral cavity, pharynx, larynx, paranasal sinuses, major salivary glands, thyroid gland, esophagus, stomach, small intestine, colon, colorectum, anal canal, liver, gallbladder, extrahepatic bile ducts, ampulla of Vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma or Kaposi's sarcoma.

139. The method according to claim 57, for treating or inhibiting tumor formation, primary tumors, tumor progression or tumor metastasis.

140. The method according to claim 60, wherein said cell proliferative disease or disorder is psoriasis, hypertrophic scars, acne or sclerosis/scleroderma.
